(19) United States
(12) Patent Application Publication
(10) Pub. No.: US 2014/0162370 A1
(43) Pub. Date: Jun. 12, 2014

(54) URINE BIOMARKERS FOR NECROTIZING ENTEROCOLITIS AND SEPSIS

(71) Applicant: The Board of Trustees of the Leland Stanford Junior University, Palo Alto, CA (US)

(72) Inventors: Bruce Xuefeng Ling, Palo Alto, CA (US); Karl G. Sylvester, Los Altos, CA (US); R. Lawrence Moss, New Albany, OH (US)

(21) Appl. No.: 14/094,509

(22) Filed: Dec. 2, 2013

Related U.S. Application Data
(63) Continuation-in-part of application No. PCT/US12/42275, filed on Jun. 13, 2012.
(60) Provisional application No. 61/496,684, filed on Jun. 14, 2011.
(60) Provisional application No. 61/732,098, filed on Nov. 30, 2012.

(51) Int. Cl. G01N 33/68

(52) U.S. Cl. CPC G01N 33/6893
USPC 436/86

(57) ABSTRACT

Aspects of the invention include methods, compositions, and kits for diagnosing Necrotizing Enterocolitis (NEC), for diagnosing sepsis, for providing a prognosis for a patient with NEC, and for predicting responsiveness of a patient with NEC to medical intervention. These methods find use in a number of applications, such as diagnosing and treating infants who are suspected of having NEC, intestinal perforation (IP), or sepsis.

### CROSS REFERENCE TO RELATED APPLICATIONS

Pursuant to 35 U.S.C. §119 (e), this application claims priority to the filing date of U.S. Provisional Patent Application Ser. No. 61/732,098, filed Nov. 30, 2012 and PCT Application No. PCT/US2012/042275, filed Jun. 13, 2012, which claims priority to U.S. Provisional Patent Application Ser. No. 61/496,684, filed Jun. 14, 2011; the full disclosures of which are herein incorporated by reference.

### GOVERNMENT RIGHTS

This invention was made with Government support under contract RR025742 awarded by the National Institutes of Health. The Government has certain rights in this invention.

### FIELD OF THE INVENTION

This invention pertains to the fields of necrotizing enterocolitis and sepsis.

### BACKGROUND OF THE INVENTION

Necrotizing enterocolitis (NEC), intestinal perforation (IP) and sepsis are three life-threatening gastrointestinal diseases among neonates and together constitute a leading cause of overall morbidity and mortality in premature newborns. However, there is considerable overlap in the early clinical presentation of NEC, IP and sepsis in newborns. Furthermore, while half of NEC-affected infants will recover with medical therapy alone (the M class), 30-50% develop a progressive form of the disease (Progressive Necrotizing Enterocolitis) that requires surgery (the S class) to prevent mortality. Currently utilized clinical parameters including laboratory tests and diagnostic imaging fail to capture the nuanced differences between these entities during their onset and progression. Protein biomarkers detectable in clinically available specimens would provide the needed molecular diagnostic and prognostic “fingerprint” against which we can begin to measure various interventions. Such biomarkers could be used to improved methods for diagnostic and prognostic class prediction in NEC, IP and sepsis, and to improve predictions on responsiveness to known and new therapies. The present invention addresses these issues.

U.S. Application No. 2009/0191551 teaches using the level of secretor antigens in a biological fluid as a marker to predict the risk of developing NEC. Thuijls G, et al. (2010) Noninvasive markers for early diagnosis and determination of the severity of necrotizing enterocolitis. Ann Surg. 251(6):1174-80, discusses using I-FABP and claudin-3 protein levels in urine and calprotectin protein levels in fecal matter as diagnostic markers of NEC, and I-FABP protein levels in urine as a prognostic marker of disease severity. Evennett N, et al. (2009) A systematic review of serologic tests in the diagnosis of necrotizing enterocolitis. J Pediatr Surg. 44(11):2192-201 is a review of publications that were deemed by the authors to be potentially relevant to diagnostic performance of serological tests in NEC. Young C, et al. (2009) Biomarkers for infants at risk for necrotizing enterocolitis: clues to prevention? Pediatr Res. 65(5 Pt 2):91R-97R is a review that discusses the potential value of genomic and proteomic studies of NEC in the identification of biomarkers for early diagnosis and targeted prevention of this disease.

### SUMMARY OF THE INVENTION

Aspects of the invention include methods, compositions, and kits for diagnosing Necrotizing Enterocolitis (NEC), for diagnosing sepsis, for providing a prognosis for a patient with NEC, and for predicting responsiveness of a patient with NEC to medical intervention. These methods find use in a number of applications, such as diagnosing and treating infants who are suspected of having NEC, intestinal perforation (IP), or sepsis. These and other objects, advantages, and features of the invention will become apparent to those persons skilled in the art upon reading the details of the compositions and methods as more fully described below.

In some aspects of the invention, methods are provided for diagnosing NEC. In these methods, a NEC-Dx signature is obtained for a patient, where a NEC-Dx signature comprises the quantitative data on the expression level of one or more NEC-Dx genes, i.e. genes that are differentially expressed in patients having NEC as compared to, e.g., unaffected individuals or individuals having sepsis. The NEC-Dx signature is then compared to a reference NEC-Dx signature, and the results of this comparison are employed to provide a diagnosis of NEC to the patient. In some embodiments, the one or more NEC-Dx genes is selected from the group consisting of SAP1, PEDF, Q6ZUQ4, OBFC2B, COL11A2, NBEAL2, GRASP, HUWE1, COL1A2, HOXD3, DSG4, KRTAP5-11, Y1020, FGA, UMOD CTAPIII/PPBP, SAA1, B2M, TTR, OSTP/OPN, APOA4, C08G, ANGT, FIBA, PROF1, PLSL, LMAN2, CST3, and RET4/RBP4, where the differential expression of one or more of these genes is diagnostic for NEC. In certain embodiments, the amount of more than one gene product, i.e. a panel of genes, is employed. In some such embodiments, the panel of interest in diagnosing NEC, i.e. distinguishing an individual having NEC from, e.g. a healthy individual or an individual having sepsis, is a panel comprising the genes CST3, PEDF, and RET4/RBP4.

In certain embodiments, the NEC-Dx signature is obtained by detecting the amount of protein or peptide in a body fluid that is encoded by one or more NEC-Dx genes to arrive at a NEC-Dx protein signature. In certain embodiments, the body fluid is urine. In certain embodiments, the patient is suspected of having NEC, intestinal perforation (IP), or sepsis. In some embodiments, the method further comprises obtaining an NEC clinical score. In such embodiments, the NEC-Dx signature and NEC clinical score are compared to an NEC-Dx signature and NEC clinical score from a reference, and the results of both comparisons are employed to provide a diagnosis of NEC.

In some aspects of the invention, methods are provided for diagnosing sepsis in a patient. In these methods, a sepsis-Dx signature is obtained from the patient, where a sepsis-Dx signature comprises the quantitative data on the expression level of one or more sepsis-Dx genes, i.e. genes that are differentially expressed in patients having sepsis as compared to, e.g., healthy individuals or individuals having NEC. The sepsis-Dx signature is then compared to a reference sepsis-Dx signature, and the results of this comparison are employed to provide a diagnosis of sepsis to the patient. In some embodiments, the one or more sepsis-Dx genes are selected from the group consisting of ftsy, PROC, MAP1B, CSN5, A2ML1, CST3, FGA, PEDF, and VASN, where differential expression of one or more of these genes is diagnostic of sepsis. In certain embodiments, the amount of more than one gene product, i.e. a panel of genes, is employed to obtain the sepsis-Dx signature. In some such embodiments, the panel of interest in diagnosing sepsis, i.e. distinguishing an individual having sepsis from, e.g. a healthy individual or an individual having NEC, is a panel comprising the genes A2ML1, CST3, FGA, and VASN. In other such embodiments, the panel of interest in diagnosing sepsis, i.e. distinguishing an individual having sepsis from, e.g. a healthy individual or an individual having NEC, is a panel comprising the genes CST3, PEDF, and VASN.

In certain embodiments, the sepsis-Dx signature is obtained by detecting/measuring the amount of protein or peptide in a body fluid that is encoded by sepsis-Dx genes to arrive at a sepsis-Dx protein signature. In certain embodiments, the body fluid is urine. In certain embodiments, the patient is suspected of having NEC, intestinal perforation (IP), or sepsis. In some embodiments, a sepsis clinical score is also obtained, the sepsis-Dx signature and the sepsis clinical score are compared to a sepsis-Dx signature and a sepsis clinical score from a reference, and the results of both comparisons are employed to provide a sepsis diagnosis to the patient.

In some aspects of the invention, methods are provided for providing a prognosis for a patient with NEC, or for predicting responsiveness of an NEC patient to medical therapy versus surgical intervention. In these methods, an NEC-M/S signature is obtained for a urine sample from the patient, where the NEC-M/S signature comprises quantitative data on the level in a body fluid of protein or peptide thereof encoded by one or more NEC-M/S genes, NEC-M/S genes being genes that are differently expressed in medical NEC (that is, NEC that is responsive to medical intervention) versus surgical NEC (that is, NEC that will require treatment by surgery). In some embodiments, the one or more NEC-M/S genes are selected from the group consisting of Q6ZUQ4, OBFC2B, COL11A2, NBEAL2, GRASP, HUWE1, COL1A2, HOXD3, DSG4, KRTAP5-11, Y1020, FGA, UMOD, OSTP/OPN, APOA4, CO8G, SAP1, ANGT, CD14, FIBA, PROF1, PEDF, PLSL, LMAN2, CST3, RET4/RBP4, A2ML1, and VASN. In some embodiments, the gene is FGA, and the amount of FGA is detected by detecting an FGA peptide selected from the group consisting of DEAGSEADHEGTHSTKR, DEAGSEADHEGTHSTKRG, and DEAGSEADHEGTHSTKR-GHAKSRPV. In some embodiments, the amount of more than one gene product, i.e. a panel of genes, is employed to obtain the NEC-M/S signature. In some such embodiments, the panel of interest comprises or consists of the genes A2ML1, CD14, CST3, PEDF, RET4, and VASN.

The NEC-M/S signature is then compared to a reference NEC-M/S signature, and the results of this comparison are employed to provide a prognosis for the patient or to predict the responsiveness of the patient to medical therapy. In some embodiments, the method also provides for making a diagnosis of NEC. In other embodiments, the patient is known to have NEC prior to performing the method.

In some embodiments, an NEC clinical score is also obtained. In some such embodiments, the NEC-M/S signature and the NEC clinical score are compared to a NEC-M/S signature and an NEC clinical score from a reference, and the results of both comparisons are employed to provide a prognosis to the patient or to predict the responsiveness of the patient to medical treatment.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-D. Clinical parameters classify only 63.6% of NEC patients correctly. Using clinical parameters, linear discriminant analysis was performed with training data from NEC M (n=30) and S (n=17) samples. The trained LDA model was then tested with testing data from NEC M (n=13) and S (n=9) samples. Estimated probabilities for the training (left) and testing data (right) are plotted (panel A). Samples are partitioned by the true class (upper) and predicted class (lower). The classification results from training (panel B) and testing sets (panel C) are shown as 2×2 contingency tables. Fisher exact test was used to measure P values of the 2×2 table. (D) Unsupervised hierarchical clustering trees based on the clinical parameters.

FIG. 2A-B. Eleven clinical parameters were selected to classify NEC M and S patients by linear discriminant analysis (LDA). (A) 11 clinical parameters (Mann Whitney U test P value <0.1) and the corresponding absolute value (ABS) of the first linear discriminant (LD1) from the LDA. (B) Using these 11 clinical parameters, a LDA model was trained with NEC M (n=30) and S (n=17) training samples and tested with data from NEC M (n=13) and S (n=9) samples. Estimated probabilities for the training (left) and testing data (right) are plotted. Samples are partitioned by the true class (upper) and predicted class (lower).

FIG. 2C-E. Eleven clinical parameters were selected to classify NEC M and S patients by linear discriminant analysis (LDA). (C, D) The classification results from the training and testing sets are shown as 2×2 contingency tables. Fisher exact test was used to measure P values of the 2×2 tables. (E) ROC analysis of the classification performance of the LDA model of the 11 clinical parameters.

FIG. 3A-B. Unsupervised clustering and pathway analyses of the MSMS identified urine peptides differentiating NEC M (n=17) and S (n=11) subjects. (A) Heatmap display of unsupervised clustering analyses of expression of the top 473 urine peptides ranked by significant analyses comparing NEC M and S samples. Manual review of the feature clusters into I, II, Ill groups. (B). Data mining software (Ingenuity Systems, www.ingenuity.com, CA) was used with these differential urine peptides' parent proteins to identify and calculate the significance of the gene ontology groups and relevant canonical signaling pathways associated with NEC progression.

FIG. 3C. Overlapping urine peptides found differentiating NEC M, S and Post S groups. m/z: Mass to charge ratio. z: Peptide charge. Relative abundance: the nearest shrunken centroid values have been utilized to represented the relative abundance of the peptide biomarkers in either NEC M or S or Post S patient class with the Color Scale conditional formatting. P*: hydroxyproline.

FIG. 4A-B. Biomarker discovery and validation. (A) Box and whisker plots of various feature sizes to distinguish the medical necrotising enterocoiitis (NEC) and surgical NEC classes. Boxes contain 50% of values falling between the 25th and 75th percentiles; the horizontal line within the box represents the median value and the ‘whisker’ lines extend to the highest and lowest values. (B) Unsupervised hierarchical cluster analysis with heat map plotting demonstrating the association of NEC disease status with the abundance pattern of 36 peptide candidate biomarkers.

FIG. 4C. Relative abundance of the 36 urine peptide abundance by the nearest shrunken centroid values in either NEC M or S patient class with the Color Scale conditional formatting. m/z: Mass to charge ratio. z: Peptide charge. P*: hydroxyproline. The significance of each urine peptide biomarker in differentiating NEC M from S groups was quantified by Mann-Whitney U test and Student T test P values.

FIG. 4D. Summarized results of PANTHER database pathway analysis for the 36-peptide candidate biomarkers.

FIG. 5A. Significant analysis of NEC M and S subjects found a 30-plasma-protein biomarker panel. (A). Goodness of separation analysis to select a panel of 48 spectral peaks (red asterisk labeled) for the NEC progression analysis. Using 1528 different spectra peak data from NEC M and S sets, as indicated, various classifiers of different panel size (feature #) were tested for their goodness of separation between NEC M (green) and NEC S (red) as shown by the box-whisker graphs. Boxes contain the 50% of values falling between the 25th and 75th percentiles; the horizontal line within the box represents the median value and the “whisker” lines extend to the highest and lowest values.

FIG. 5B. Spectral analysis of the 48 spectral peak found 30 unique plasma proteins. Relative abundance of the 30 proteins were represented by the nearest shrunken centroid values in either NEC M or S patient class with the Color Scale conditional formatting. MW: molecular weight. The significance of each plasma protein in differentiating NEC M from S groups was quantified by Mann-Whitney U test and Student T test P values.

FIG. 5C. Heatmap display of unsupervised clustering analyses of expression of the 30 plasma protein biomarkers.

FIG. 6A-B. Performance evaluation in differentiating NEC 13 M and 11 S subject via (A) 11 clinical parameter based biomarker panel; and (B) 36 urine peptide based biomarker panel. Each of the unsupervised clustering results of the NEC M and S subjects are shown as a 2×2 contingency table. Fisher exact test was used to measure P value quantifying the biomarker panel's capability in NEC progression prediction.

FIG. 6C-D. Performance evaluation in differentiating NEC 13 M and 11 S subject via_(C) 30 plasma protein based biomarker panel; and (D) an integrative panel combining all 11 clinical parameters, 36 urine peptides and 30 plasma proteins. Each of the unsupervised clustering results of the NEC M and S subjects are shown as a 2×2 contingency table. Fisher exact test was used to measure P value quantifying the biomarker panel's capability in NEC progression prediction.

FIG. 7A-B. Analysis integrating clinical, urine peptide and plasma protein panels derived a biomarker panel of 15 urine peptides and 3 plasma proteins, that predicts NEC progression with high sensitivity and specificity. (A). Goodness of separation and (B) false discovery rate (FDR) analyses chose 18 features from a total of 77 biomarkers (11 clinical parameters, 36 urine peptides and 30 plasma proteins) as the optimal biomarker panel for NEC progression prediction.

FIG. 7C. Relative abundance of the 15 urine peptide and 3 plasma protein abundance in FIG. 7A-B by the nearest shrunken centroid values in either NEC M or S patient class with the Color Scale conditional formatting. For urine peptides, MW=MH+−1=m/z−1.

FIG. 7D-E. (D) Heatmap display of unsupervised clustering analyses of expression of the 18 (15 urine peptides and 3 plasma proteins) biomarkers. The clustering result is shown as a 2×2 contingency table. Fisher exact test was used to measure the statistical significance (P value) of the 2×2 table. (E). Supervised LDA analysis classifying NEC M and S subjects. Samples are partitioned by the true class (upper) and predicted class (lower). The LDA classification result is shown as a 2×2 contingency table. Fisher exact test was used to measure the statistical significance (P value) of the 2×2 table.

FIG. 7F. ROC analysis of the integrative biomarker panel in discriminating NEC M and S. AUC: area under the curve. The dotted curve is the vertical average of the 500 bootstrapping ROC curves and the boxes and whiskers plot the vertical spread around the average.

FIG. 8A-B. A sequential analysis of the clinical and molecular biomarker classifiers for the prediction of NEC progression. (A) NEC clinical scoring system. The samples (violet red-NEC S, sea green-NEC M), sorted by their clinical NEC scores, were grouped into low, intermediate, and high-risk groups. Each particular sample's risk of being NEC S was quantified as the proportion of all NEC S samples with score less than that sample's clinical score in all NEC S samples. (B) Sequential stratification of the NEC subjects using clinical and molecular based classifiers. The molecular based classification result is shown as a 2×2 contingency table. Fisher exact test was used to measure the statistical significance (P value) of the 2×2 table.

FIG. 9. Clinical parameter-based diagnostic algorithm. (A) Density plots of medical necrotising enterocolitis (NEC) and surgical NEC infants' outcome scores based on clinical parameters. The area outside the dotted vertical lines represents prediction with 95% confidence, while the area between the lines represents the ‘indeterminate’ prediction. The percentage of infants with indeterminate predictions in the training and testing cohorts were 42.4% and 40.1%, respectively. (B) The performance of the linear discriminant analysis (LDA) model in outcome prediction by receiver-operator characteristic (ROC) area under the curve (AUC) analysis.

FIG. 10A-B. Biomarker discovery and validation. (A) Validation of the peptide biomarkers by LC-MALDI in the biomarker discovery cohort (medical NEC, n=17; surgical NEC, n=10) and the (B) Biomarker validation cohort (medical NEC, n=27; surgical NEC, n=10). The whisker plots summarize the quantitative mass spec validation results in each of the depicted cohorts. Fishers exact test indicates the significance of separation between the medical NEC and surgical NEC infants. FGA peptide sequences: FGA1826 DEAGSEADHEGTHSTKR; FGA1883 DEAGSEADHEGTHSTKRG; FGA2659 DEAGSEADHEGTHSTKRGHAKSRPV.

FIG. 10C. Statistical performance of FGA classifiers upon combining the discovery (FIG. 10A) and validation (FIG. 10B) sets (medical NEC, n=44; surgical NEC, n=20) of the three FGA peptides in urine.

FIG. 11. Receiver-operator characteristic (ROC) analysis and area under the curve (AUC) for the validated biomarkers. (A) Discovery cohort. (B) Validation cohort.

FIG. 12A-B. Performance of necrotising enterocolitis (NEC) Outcome Risk Stratification Algorithms. (A) Clinical parameter-based algorithm, 39% of all infants remain in the indeterminate group (n=25/64) represented by the area between the horizontal dotted lines. (B) Ensemble algorithm integrating clinical parameters with the fibrinogen (FGA) urine peptide biomarkers. The arrow indicates the five infants with pneumoperitoneum at presentation (assigned arbitrarily) with high prediction scores.

FIG. 13. Western blot analysis of urine CD14

FIG. 14. Correlation of CD14 LCMS spectral counts and CD14 Western blot gel band intensity for infants in the Sepsis, Medical NEC, and Surgical NEC groups.

FIG. 15A. Single analyte biomarker's performances in discriminating NEC M and S were analyzed by ROC analysis as described for FIG. 15D.

FIG. 15B. Single analyte biomarker's performances in discriminating NEC and control were analyzed by ROC analysis as described for FIG. 15D.

FIG. 15C. Single analyte biomarker's performances in discriminating NEC and sepsis were analyzed by ROC analysis as described for FIG. 15D.

FIG. 15D. Single analyte biomarker's performances in discriminating sepsis and control classes were analyzed by ROC analysis. The Y axis is the sensitivity and X axis is the 1—specificity. The red dot represents the point of optimized sensitivity and specificity and is listed under each ROC plot. 500 testing data sets were generated by bootstrapping methods from the ELISA data and were used to derive estimates of standard error and confidence intervals for the ROC analyses. The plotted ROC curve represents the vertical average of the 500 bootstrapping runs, and the box and whisker plots show the vertical spread around the average.

FIG. 16. Biomarker panels for Medical NEC versus Surgical NEC, NEC versus Control, NEC versus Sepsis, Sepsis versus Control classifications. Sample panel score was defined as the ratio of the geometric mean of the up-regulated panel markers' assay results and those of the down-regulated panel markers' assay results. SD: standard deviation; IQR: inter quartile range.

FIG. 17. Biomarker panel ROC curves; AUC, area under the curve. The “cut-off” points along the ROC curves are labeled in red indicating the best sensitivity and specificity coordinates. Panel 1 (NEC vs. Sepsis) consists of three proteins: CST3, PEDF and RET4. Panel 2 (Medical NEC vs. Surgical NEC) consists of six proteins: A2ML1, CD14, CST3, PEDF, RET4, and VASN. Panel 3 (NEC vs. Control) consists of three proteins: CST3, PEDF and RET4. Panel 4 (Sepsis vs. Control) consists of 4 proteins: A2ML1, CST3, FGA, and VASN.

FIG. 18. Bottom-up urine proteomics discovered an eleven-protein biomarker panel effectively discriminate NEC M from S subjects. 71 NEC (47 M and 24 S) urine samples were collected and subjected to mass spectrometry (MS) based urine proteome profiling using a bottom-up approach. Each proteome was fragmented by trypsin digestion. Full mass spectrometry scan was acquired on an LTQ FTMS, which was followed by MS/MS analysis. Protein identification was performed by searching Swiss-Prot database. Quantification of proteins in different samples was done by means of spectral counting, implementing the recent S1N algorithm (Sardiu, 2010). From the MSMS protein identifications, a separate list of proteins was created for each sample, and the lists were then compared to find differential expressed proteins. For any given protein, the significance of the relative abundance between NEC M and S groups was computed by Student's T test. Urine proteins with low P values discriminating NEC and Sepsis were explored by exploratory box-whisker plot analysis.

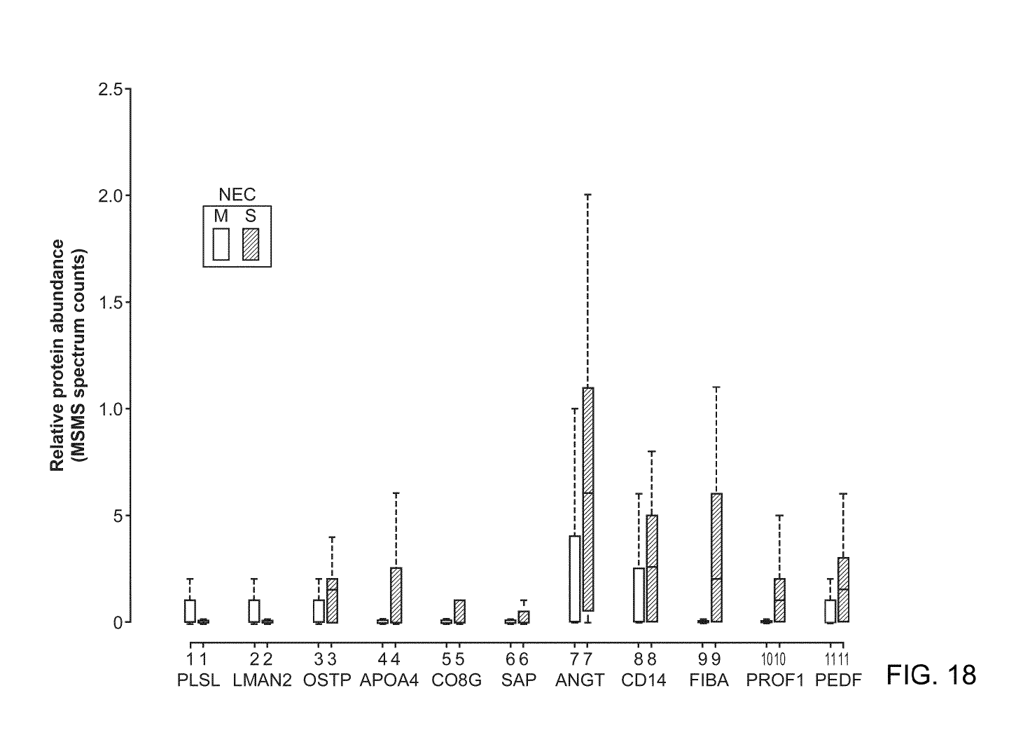

FIG. 19. Statistical analysis of the eleven-urine-protein NEC M/S biomarker panel. (A) The discriminant probabilities for each sample were calculated from the linear discriminant analysis. The maximum estimated probability for each of the wrongly classified samples is marked with an arrow. (B). A modified 2×2 contingency table was used to the calculated the percentage of classification that agreed with clinical diagnosis for the panel. P value was calculated with Fisher's exact test. (C). The discriminant analysis-derived prediction scores for each sample were used to construct a receiver operating characteristic (ROC) curve. 500 testing data sets, generated by bootstrapping, from the NEC and sepsis data were used to derive estimates of standard errors and confidence intervals for our ROC analysis. The plotted ROC curve is the vertical average of the 500 bootstrapping runs, and the box and whisker plots show the vertical spread around the average.

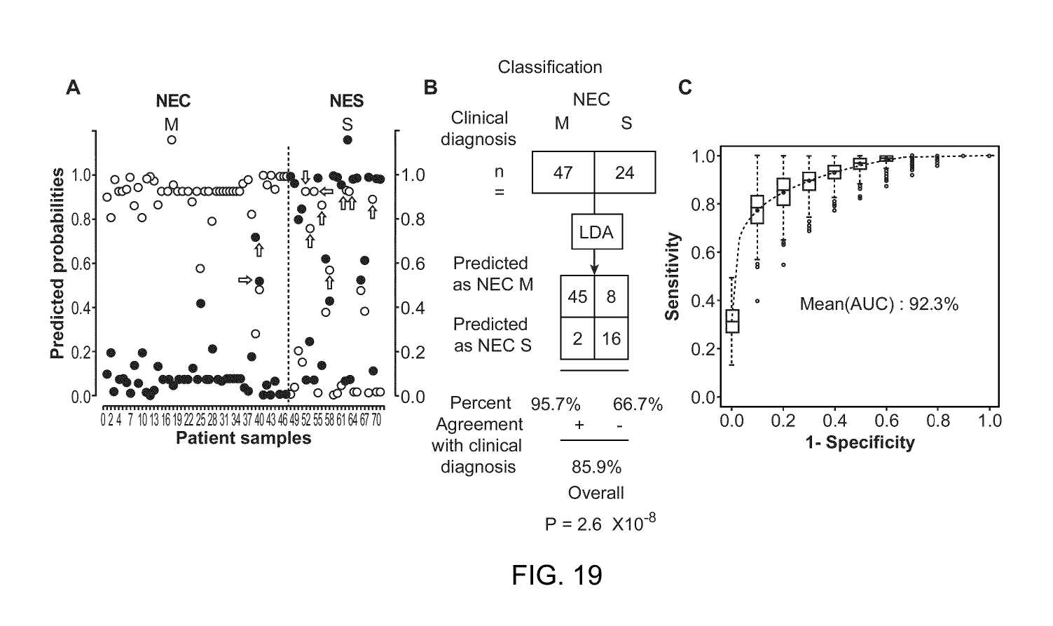

FIG. 20. Bottom-up urine proteomics discovered a seven-protein biomarker panel effectively discriminate NEC from Sepsis subjects. 71 NEC and 13 Sepsis urine samples were collected and subjected to mass spectrometry (MS)-based urine proteome profiling using a bottom-up approach. Each proteome was fragmented by trypsin digestion. Full mass spectrometry scan was acquired on an LTQ FTMS, which was followed by MS/MS analysis. Protein identification was performed by searching Swiss-Prot database. Quantification of proteins in different samples was done by means of spectral counting, implementing the recent S1N algorithm {Sardiu, 2010}. From the MSMS protein identifications, a separate list of proteins was created for each sample, and the lists were then compared to find differential expressed proteins. For any given protein, the significance of the relative abundance between NEC and Sepsis groups was computed by Student's T test. Urine proteins with low P values discriminating NEC and Sepsis were explored by exploratory box-whisker plot analysis.

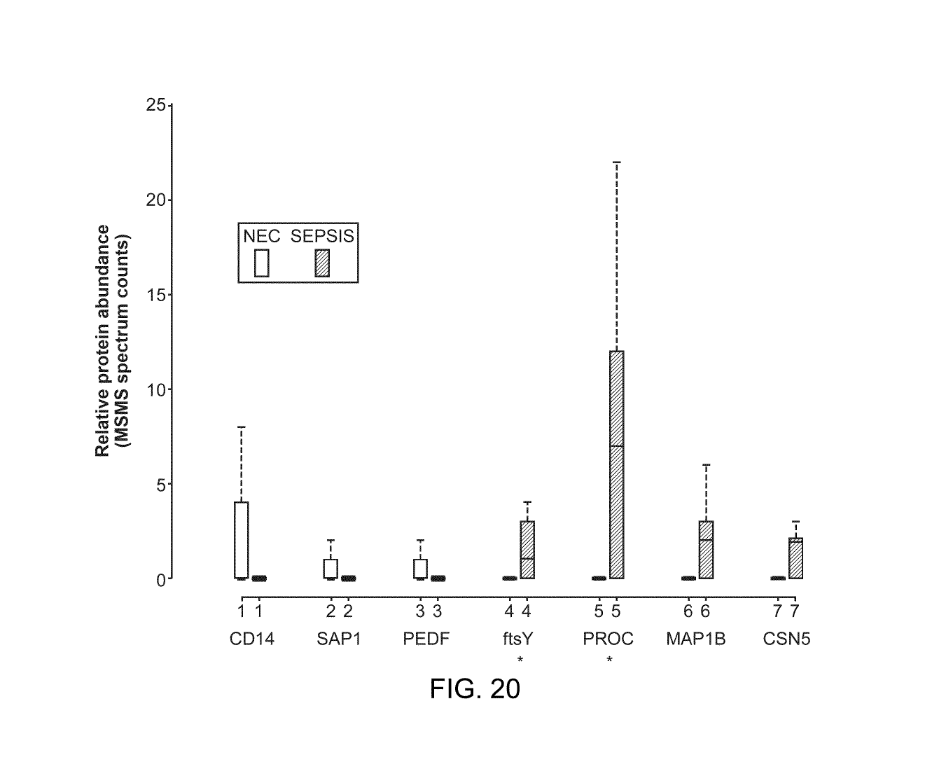

FIG. 21. Statistical analysis of the seven-urine-protein NEC/sepsis biomarker panel. (A) The discriminant probabilities for each sample were calculated from the linear discriminant analysis. The maximum estimated probability for each of the wrongly classified samples is marked with an arrow. (B). A modified 2×2 contingency table was used to the calculated the percentage of classification that agreed with clinical diagnosis for the panel. P value was calculated with Fisher's exact test. (C). The discriminant analysis-derived prediction scores for each sample were used to construct a receiver operating characteristic (ROC) curve. 500 testing data sets, generated by bootstrapping, from the NEC and sepsis data were used to derive estimates of standard errors and confidence intervals for our ROC analysis. The plotted ROC curve is the vertical average of the 500 bootstrapping runs, and the box and whisker plots show the vertical spread around the average.

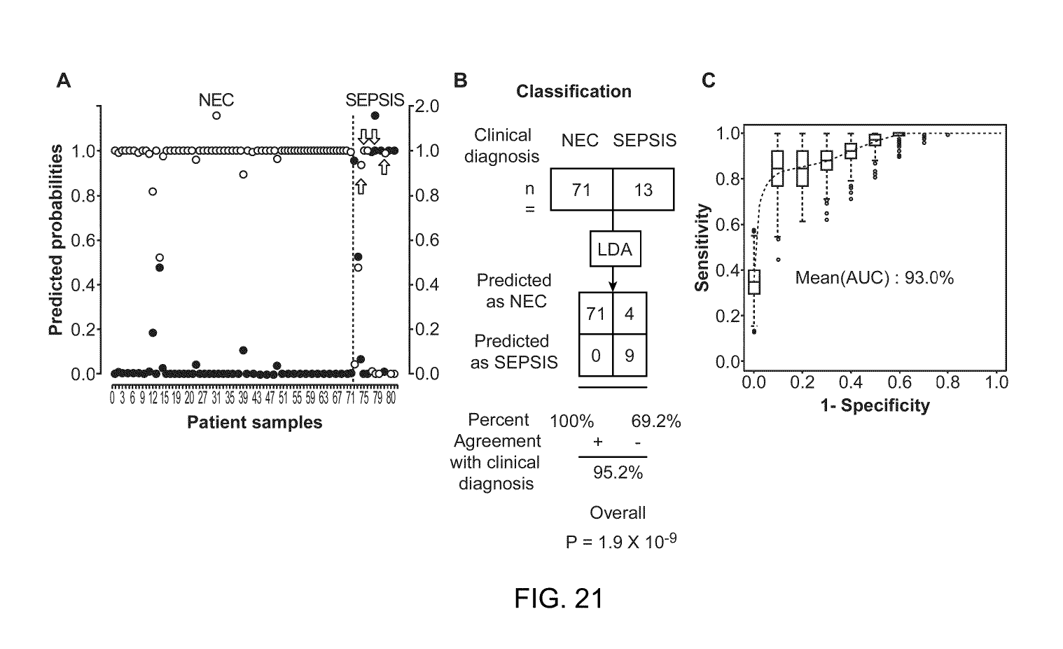

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech. Methodologies for the discovery of urinary peptide biomarkers are detailed in X. B. Ling et al., Advances in Clinical Chemistry 51, 181, 2010.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Aspects of the invention include methods, compositions, and kits for diagnosing Necrotizing Enterocolitis (NEC), for diagnosing sepsis, for providing a prognosis for a patient with NEC, and for predicting responsiveness of a patient with NEC to medical intervention. These methods find use in a number of applications, such as diagnosing and treating infants who are suspected of having NEC, IP, or sepsis. These and other objects, advantages, and features of the invention will become apparent to those persons skilled in the art upon reading the details of the compositions and methods as more fully described below.

The term Necrotizing Enterocolitis, or NEC, is used herein to describe the gastrointestinal condition in which a segment of the intestine becomes necrotic; in some instances, the intestinal region perforates, causing peritonitis and often free intra-abdominal air. Infection and inflammation of the gut are hallmarks of the condition, along with abdominal distention, blood in the stool, diarrhea, feeding intolerance, lethargy, temperature instability, and vomiting. There are two classes of NEC: M, for “medical”, class; and S, for “surgical”, class.

The terms “medical class NEC”, “M class NEC”, “NEC-M”, or “non-progressive NEC” are used interchangeably herein to describe the class of NEC that is typically responsive to medical therapies, e.g. stage I, stage II, and in some instances stage III of Bell's criteria (Table 1 below). Medical therapy includes, for example, broad spectrum antibiotics for 3-14 days, accompanied intravenous fluids, total parenteral fluids (TPN) and NPO (nothing by mouth).

The terms “surgical class NEC”, “S class NEC”, “NEC-S”, or “progressive NEC” are used interchangeably herein to describe the class of NEC that requires surgical intervention, e.g. stage IIIB of Bell's criteria (Table 1 below). In this surgery, gangrenous bowel is resected, and ostomies for intestinal stream diversion are created. With resolution of sepsis and peritonitis, intestinal continuity can be reestablished several weeks or months later.

The terms “focal intestinal perforation” (FIP), “spontaneous intestinal perforation” (SIP), or “intestinal perforation” (IP) are used interchangeably herein to describe an isolated intestinal perforation that, unlike NEC, is not accompanied by gross necrosis of the tissue. In FIP, the gestational age is significantly lower than in NEC (approx. 24 weeks versus 27 weeks for NEC), the incidence of coexistent respiratory distress syndrome (RDS) is higher (88% versus 37% for NEC), and the age of onset is younger (approx. 7.3 days versus approx. 7.9 days for NEC). See, e.g. Okuyama et al. (2002) Pediatr Surg Int 18:704-706, the disclosure of which is incorporated herein by reference.

The term “sepsis” is used herein to describe a bacterial infection in the context of fever of greater than 38° C. (100.4° F.). Blood pressure drops, resulting in shock. Major organs and systems, including the kidneys, liver, lungs, and central nervous system, stop functioning normally. Infection is typically confirmed by a blood culture that reveals bacteria, blood gases that reveal acidosis, kidney function tests that are abnormal, a platelet count that is lower than normal, and/or a white blood cell count that is lower or higher than normal. Other indications of sepsis include a blood differential that shows immature white blood cells, the presence of higher than normal amounts of fibrin degradation products in the blood, and a peripheral smear that shows a low platelet count and destruction of red blood cells. The treatment is typically antibiotics delivered intravenously. In infants, sepsis may be classified as “early onset” (within the first 7 days of birth), which usually results from organisms acquired intrapartum, and “late onset” (more than 7 days after birth), in which the infection is usually by organisms from the environment.

“Diagnosis” as used herein generally includes a prediction of a subject's susceptibility to a disease or disorder, determination as to whether a subject is presently affected by a disease or disorder, and prognosis of a subject affected by a disease or disorder (e.g., identification of disease states, stages of the disease, likelihood that a patient will die from the disease), and the use of therametrics (e.g., monitoring a subject's condition to provide information as to the effect or efficacy of therapy). “Prediction of a subject's responsiveness to treatment” for the disease or disorder generally includes the prediction of responsiveness (e.g., positive response, a negative response, no response at all to, e.g., medical treatment, surgical treatment), and prognosis in view of that predicted responsiveness.

The terms “biomarker”, “gene product” and “expression product” are used interchangeably herein to refer to the RNA transcription products (transcripts) of the gene, including mRNA, the polypeptide translation products of such RNA transcripts, and peptide fragments thereof. A gene product can be, for example, an unspliced RNA, an mRNA, a splice variant mRNA, a microRNA, a fragmented RNA, a polypeptide, a post-translationally modified polypeptide, a splice variant polypeptide, a peptide, etc.

The term “RNA transcript” as used herein refers to the RNA transcription products of a gene, including, for example, mRNA, an unspliced RNA, a splice variant mRNA, a microRNA, and a fragmented RNA.

The term “polypeptide” as used herein and as it is applied to a gene refers to the amino acid product encoded by a gene, including, for example, full length gene product, splice variants of the full length gene product, and fragments of the gene product, e.g. peptides.

The term “expression level” as used herein and as it is applied to a gene refers to the amount of a gene product in a sample, e.g. the normalized value determined for the amount of RNA transcribed from a gene, or the normalized value determined for the amount of polypeptide/protein encoded by the gene or peptide fragment thereof. Normalization of the expression level(s) of a gene may be by any well-understood method in the art, e.g. by comparison to the expression of a selected housekeeping gene(s), by comparison to the expression of genes across a whole dataset, etc.

The term “expression signature” is a representation of the expression levels of one or more genes of interest, more usually two or more genes of interest, and comprises the quantitative data on the expression levels of these one or more genes of interest. Examples of expression signatures include expression profiles, e.g. RNA profiles and protein profiles, and expression scores, e.g. RNA scores and protein scores.

The term “expression profile” as used herein refers to the normalized expression level of one or more genes of interest, more usually two or more genes of interest, in a patient sample. By “RNA expression profile”, or simply “RNA profile”, of a patient sample it is meant the normalized expression level of the one or more genes in a patient sample as determined by measuring the amount of RNA transcribed from the one or more genes. By “protein expression profile”, or simply “protein profile”, of a patient sample it is meant the normalized expression level of the one or more genes in a patient sample as determined by measuring the amount of amino acid product encoded by a gene.

The term “expression score” as used herein refers to a single metric value that represents the sum of the weighted expression levels of one or more genes of interest, more usually two or more genes of interest, in a patient sample. Weighted expression levels are calculated by multiplying the normalized expression level of each gene by its “weight”, the weight of each gene being determined by analysis of a reference dataset, or “training set”, e.g. the datasets provided in the examples section below, e.g. by Principle Component Analysis (PCA), Linear discriminant analysis (LDA), Fishers linear discriminant analysis, and the like, as are known in the art. Thus, for example, when PCA is used, the expression score is the weighted sum of expression levels of the genes of interest in a sample, where the weights are defined by their first principal component as defined by a reference dataset. By “RNA expression score”, or simply “RNA score”, of a patient sample it is meant the normalized expression level of the one or more genes in a patient sample as determined by measuring the amount of RNA transcribed from the one or more genes. By “protein expression profile”, or simply “protein profile”, of a patient sample it is meant the normalized expression level of the one or more genes in a patient sample as determined by measuring the amount of amino acid product encoded by a gene.

An “NEC-Dx gene” is a gene that is differentially expressed in individuals having NEC relative to individuals that are not affected with NEC.

An “NEC-Dx expression signature”, or more simply, “NEC-Dx signature”, is a representation of the expression levels of one or more NEC-Dx genes, and comprises the quantitative data on the amount of RNA, protein, or peptide fragment thereof encoded by these one or more NEC-Dx genes. An “NEC-Dx RNA signature” comprises the quantitative data on the amount of RNA transcribed by one or more NEC-Dx genes. An “NEC-Dx protein signature” comprises the quantitative data on the amount of polypeptide/protein encoded by the one or more NEC-Dx genes and/or peptides thereof. An NEC-Dx signature may be in the form of an expression profile or an expression score, as discussed above.

A “sepsis-Dx gene” or “Sepsis Diagnosis gene” is a gene that is differentially expressed in individuals having sepsis relative to individuals that are not affected with sepsis.

A “sepsis-Dx expression signature”, or more simply, a “sepsis-Dx signature”, is a representation of the amount of RNA, protein, or peptide fragment thereof encoded by one or more sepsis-Dx genes, and comprises the quantitative data on the expression levels of these one or more genes. A “sepsis-Dx RNA signature” comprises the quantitative data on the amount of RNA transcribed by one or more sepsis-Dx genes. A “sepsis-Dx protein signature” comprises the quantitative data on the amount of polypeptide encoded by one or more sepsis genes and/or peptides thereof. A sepsis-Dx signature may be in the form of an expression profile or an expression score, as discussed above.

An “NEC-M/gene” is a gene that is differentially expressed in individuals having M class NEC relative to S class NEC or vice versa. In other words, an NEC-M/S gene is a gene that is expressed at a higher or lower level in one class of NEC versus the other. An NEC-M/S gene may be used to distinguish between M class NEC and S class NEC, e.g. to classify an NEC, to prognose a NEC, to determine a treatment for an NEC, etc.

An “NEC-M/S expression signature”, or more simply, “NEC-M/signature”, is a representation of the amount of RNA, protein, or peptide fragment thereof encoded by one or more NEC M/S genes, and comprises the quantitative data on the expression levels of these one or more genes. An “NEC-M/S RNA signature” comprises the quantitative data on the amount of RNA transcribed by one or more NEC-M/S genes. An “NEC-M/S protein signature” comprises the quantitative data on the amount of polypeptide encoded by one or more NEC-M/S genes and/or peptides thereof. An NEC-M/S signature may be in the form of an expression profile or an expression score, as discussed above.

The term “risk classification” means a level of risk (or likelihood) that a subject will experience a particular clinical outcome. A subject may be classified into a risk group or classified at a level of risk based on the methods of the present disclosure, e.g. high, medium, or low risk. A “risk group” is a group of subjects or individuals with a similar level of risk for a particular clinical outcome. Examples of NEC risk groups include M-class and the S-class.

The term “hazard ratio” means the effect of an explanatory variable on the hazard, or risk, of an event occurring. For example, using a Cox proportional hazards regression model, if a variable, e.g. an LSC score, is prognostic, its hazard rate is different in patients with a particular prognosis relative to the hazard rate of other subclasses, and the hazard ratio of the gene is not equal to 1.

The terms “individual,” “subject,” “host,” and “patient,” are used interchangeably herein and refer to any mammalian subject for whom diagnosis, treatment, or therapy is desired, particularly humans.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

Methods, compositions and kits are provided for diagnosing Necrotizing Enterocolitis (NEC) and sepsis, for providing a prognosis for a patient with NEC, and for predicting responsiveness of a patient with NEC to medical therapy. These methods find particular use in diagnosing and treating patients, e.g. infants that are suspected of having NEC, IP, or sepsis.

### Obtaining an Expression Signature.

In practicing methods of the invention, an expression signature, e.g. an NEC-Dx expression signature, a sepsis-Dx expression signature, or an NEC-M/S expression signature, is obtained for a patient that is suspected of having NEC or sepsis. Non-limiting examples of genes that may be employed as NEC-Dx genes, sepsis-Dx genes, and/or NEC-M/S genes are provided in Table 1.

TABLE 1
Genes of interest as NEC-Dx genes, sepsis-Dx genes, and/or NEC-M/S
genes. Sequences for genes are provided as Genbank Accession Entries, the disclosures of
which are specifically incorporated herein by reference.

| Gene | Gene name, aliases | Genbank Accession No. |
|---|---|---|
| CD14 | CD14 molecule | NM_000591.3 (variant 1) |
| | | NM_001040021.2 (variant 2) |
| | | NM_001174104.1 (variant 3) |
| | | NM_001174105.1 (variant 4) |
| SAP1 | SH2 domain containing 1A; SAP; | NM_002351.4 (isoform 1); |
| | SH2D1A | NM_001114937.2 (isoform 2) |
| PEDF | serpin peptidase inhibitor, clade F | NM_002615.4 |
| | (alpha-2 antiplasmin, pigment | |
| | epithelium derived factor), | |
| | member 1; SERPINF1 | |
| Q6ZUQ4 | CDNA FLJ43449 fis | Q6ZUQ4 (protein database) |
| OBFC2B | oligonucleotide/oligosaccharide- | NM_024068.3 |
| | binding fold containing 2B | |
| COL11A2 | collagen, type XI, alpha 2 | NM_080680.2 (isoform 1), |
| | | NM_080681.2 (isoform 2), |
| | | NM_080679.2 (isoform 3), |
| | | NM_001163771 (isoform 4) |
| NBEAL2 | neurobeachin-like 2 | NM_015175.1 |
| GRASP | GRP1 (general receptor for | NM_181711.2 |
| | phosphoinositides 1)-associated | |
| | scaffold protein | |
| HUWE1 | HECT, UBA and WWE domain | NM_031407.4 |
| | containing 1 | |
| COL1A2 | collagen, type I, alpha 2 | NM_000089.3 |
| HOXD3 | homeobox D3 | NM_006898.4 |
| DSG4 | desmoglein 4 | NM_001134453.1 (variant 1) |
| | | NM_177986.3 (variant 2) |
| KRTAP5- | keratin associated protein 5-11 | NM_001005405.2 |
| 11 | | |
| Y1020 | hypothetical protein Y1020 | NP_857803 |
| | [Yersinia pestis KIM]. | |
| FGA | fibrinogen alpha chain; Fib2; FIBA | NM_021871.2 (isoform α) |
| | | NM_000508.3 (isoform α-E) |
| UMOD | Uromodulin | NM_003361.2 (variant 1) |
| | | NM_001008389.1 (variant 2) |
| CTAPIII | pro-platelet basic protein | NM_002704.3 |
| | (chemokine (C-X-C motif) ligand 7; | |
| | PPBP | |
| SAA1 | serum amyloid A1 | NM_000331.4 |
| B2M | beta-2-microglobulin | NM_004048.2 |
| TTR | Transthyretin | NM_000371.3 |
| OSTP | Osteopontin; OPN; secreted | NM_001040058.1 |
| | phosphoprotein 1, SPP1, BNSP; | NM_001040058.1 |
| | BSPI; ETA-1; MGC110940 | NM_000582.2 |
| APOA4 | apolipoprotein A-IV | NM_000482.3 |
| CO8G | Complement component C8 | NM_000606.2 |
| | gamma chain; C8G | |
| ANGT | Angiotensinogen; serpin peptidase | NM_000029.3 |
| | inhibitor, clade A, member 8; AGT | |
| FIBA | Fibrinogen alpha chain; FGA | NM_000508.3 |
| | | NM_021871.2 |
| PROF1 | Profilin 1; PFN1 | NM_005022.2 |
| PLSL | Plastin-2; lymphocyte cytosolic | NM_002298.4 |
| | protein 1; LCP1 | |
| LMAN2 | lectin, mannose-binding 2 | NM_006816.2 |
| ftsY | ECK3448, b3464, JW3429 | NP_417921.1 |
| | Bacterial signal recognition particle | |
| | receptor | |
| PROC | protein C (inactivator of coagulation | NM_000312.3 |
| | factors Va and Villa) | |
| MAP1B | microtubule-associated protein 1B | NM_005909.3 |
| CSN5 | COP9 constitutive | NM_006837.2 |
| | photomorphogenic homolog | |
| | subunit 5 (Arabidopsis); COPS5 | |
| A2ML1 | alpha-2-macroglobulin-like 1 | NM_144670.4 (isoform 1) |
| | | NM_001282424.1 (isoform 2) |
| CST3 | Cystatin 3 | NM_000099.2 |
| RET4 | Retinol binding protein 4 (RBP4) | NM_006744.3 |
| VASN | Vasorin | NM_138440.2 |

In some embodiments, the subject expression signature, e.g. NEC-Dx signature, sepsis-Dx signature, or NEC-M/S signature, is a representation of the amount of RNA, protein, or peptide fragment thereof encoded by one or more of the aforementioned genes. In some embodiments, the subject expression signature, e.g. NEC-Dx signature, sepsis-Dx signature, or NEC-M/S signature, is a representation of the amount of RNA, protein, or peptide fragment thereof encoded by two or more of the aforementioned genes, i.e. a panel of the aforementioned genes, e.g. 2, 3, 4, or 5 of the aforementioned genes or more, e.g. 6, 7, 8, 9, or 10 of the aforementioned genes or more, in some cases, 11, 12, 13, 14, or 15 of the aforementioned genes or more, for example, 16, 17, 18, 19 or 20 of the aforementioned genes.

Genes of particular interest for use in arriving at a subject NEC-Dx signature include one or more of SAP1, PEDF, Q6ZUQ4, OBFC2B, COL11A2, NBEAL2, GRASP, HUWE1, COL1A2, HOXD3, DSG4, KRTAP5-11, Y1020, FGA, UMOD CTAPIII/PPBP, SAA1, B2M, TTR, OSTP/OPN, APOA4, C08G, ANGT, FIBA, PROF1, PLSL, LMAN2, CST3, and RET4/RBP4. In certain instances, the genes of interest for use in arriving at the subject NEC-Dx signature make up a panel of genes comprising or consisting of CST3, PEDF, and RET4/RBP4.

Genes of particular interest for use in arriving at a subject sepsis-Dx signature include one or more of ftsy, PROC, MAP1B, CSN5, A2ML1, CST3, FGA, PEDF, and VASN. In certain instances, the genes of interest for use in arriving at the subject sepsis-Dx signature make up a panel of genes comprising or consisting of A2ML1, CST3, FGA, and VASN. In certain instances, the genes of interest for use in arriving at the subject sepsis-Dx signature make up a panel of genes comprising or consisting of CST3, PEDF, and VASN.

Genes of particular interest for use in arriving at a subject NEC-M/S signature include one or more of Q6ZUQ4, OBFC2B, COL11A2, NBEAL2, GRASP, HUWE1, COL1A2, HOXD3, DSG4, KRTAP5-11, Y1020, FGA, UMOD, OSTP/OPN, APOA4, CO8G, SAP1, ANGT, CD14, FIBA, PROF1, PEDF, PLSL, LMAN2, CD14, CST3, RET4/RBP4, A2ML1, and VASN. In some embodiments, the one or more genes is selected from the group consisting of Q6ZUQ4, OBFC2B, COL11A2, NBEAL2, GRASP, HUWE1, COL1A2, HOXD3, DSG4, KRTAP5-11, Y1020, FGA, OSTP/OPN, APOA4, CO8G, SAP1, ANGT, CD14, FIBA, PROF1, PEDF, CD14, CST3, and RET4/RBP4, where high levels of one or more of the gene products is diagnostic of NEC-S, and low levels of one or more of the gene products is diagnostic of NEC-M. In certain embodiments, the gene is FGA, and the peptide is selected from the group consisting of DEAGSEADHEGTHSTKR, DEAGSEADHEGTHSTKRG, and DEAGSEADHEGTHSTKR-GHAKSRPV. In some embodiments, the one or more genes is selected from the group consisting of UMOD, PLSL, LMAN2, A2ML1, and VASN, where high levels of one or more of these genes is diagnostic of NEC-M, and low levels of one or more of these genes is diagnostic of NEC-S. In certain instances, the genes of interest for use in arriving at the subject NEC-M/S signature make up a panel of genes comprising or consisting of A2ML1, CD14, CST3, PEDF, RET4, and VASN.

In practicing methods of the invention, an expression signature, e.g. a NEC-Dx expression signature, a sepsis-Dx expression signature, or an NEC-M/S expression signature, is obtained for a patient. In some embodiments, the patient is suspected of having NEC or sepsis. A patient that is suspected of having NEC or sepsis is one in which historical factors, physical findings and radiological findings indicate risk for NEC or sepsis. Historical factors include, for example, feeding intolerance (defined as vomiting two or more feedings within 24 hours or any vomit containing bile, or the presence of gastric residuals of volume greater than 6 cc/kg or any aspirate containing bile), apneic/bradycardic episodes, oxygen desaturation episodes, guaiac positive, or bloody stools. Physical findings include, for example, abdominal distention, capillary refill time >2 sec, abdominal wall discoloration, or abdominal tenderness. Radiological findings include, for example, pneumatosis intestinalis, portal venous gas, Ileus, dilated bowel, pneumoperitoneum, air/fluid levels, thickened bowel walls, ascites or peritoneal fluid, or free intraperitoneal air, absent bowel sounds, hypotension, abdominal cellulitis, and right lower quadrant mass.

To obtain an expression signature, the expression level of the one or more genes of interest is measured, i.e. the expression levels of 1 or more, 2 or more, or 3 or more genes is determined, e.g. 4 or more, 5 or more, 6 or more or 7 or more genes, in some embodiments 8-15 genes, in some embodiments 16-28 genes, e.g. the expression levels of 28 or more genes is determined. The expression level is typically measured by analyzing a body fluid sample, e.g. a sample of urine, blood, or saliva, that is obtained from an individual. Usually, the sample is a urine sample. The sample that is collected may be freshly assayed or it may be stored and assayed at a later time. If the latter, the sample may be stored by any convenient means that will preserve the sample so that gene expression may be assayed at a later date. For example the sample may be freshly cryopreserved, that is, cryopreserved without impregnation with fixative, e.g. at 4° C., at −20° C., at −60° C., at −80° C., or under liquid nitrogen. Alternatively, the sample may be fixed and preserved, e.g. at room temperature, at 4° C., at −20° C., at −60° C., at −80° C., or under liquid nitrogen, using any of a number of fixatives known in the art, e.g. alcohol, methanol, acetone, formalin, paraformaldehyde, etc.

The sample may be assayed as a whole sample, e.g. in crude form. Alternatively, the sample may be fractionated prior to analysis, e.g. for a blood sample, to purify leukocytes if, e.g., the gene expression product to be assayed is RNA or intracellular protein, or to purify plasma or serum if, e.g., the gene expression product is a secreted polypeptide. Further fractionation may also be performed, e.g., for a purified leukocyte sample, fractionation by e.g. panning, magnetic bead sorting, or fluorescence activated cell sorting (FACS) may be performed to enrich for particular types of cells, thereby arriving at an enriched population of that cell type for analysis; or, e.g., for a plasma or serum sample, fractionation based upon size, charge, mass, or other physical characteristic may be performed to purify particular secreted polypeptides, e.g. under denaturing or non-denaturing (“native”) conditions, depending on whether or not a non-denatured form is required for detection. One or more fractions are then assayed to measure the expression levels of the one or more genes of interest.

The expression levels of the one or more genes of interest may be measured by measuring protein levels, i.e. peptide or polypeptide, levels or by measuring RNA levels.

For measuring protein levels, the amount or level in the sample of one or more proteins/polypeptides or peptide fragments thereof encoded by the gene of interest is determined. In such cases, any convenient protocol for evaluating protein or peptide levels may be employed wherein the level of one or more proteins or peptides in the assayed sample is determined.

While a variety of different manners of assaying for protein levels are known in the art, one representative and convenient type of protocol for assaying levels of protein or peptide fragments thereof is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hrs at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hr at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more polypeptides or peptide fragments thereof in a sample may be employed. Representative examples include but are not limited to mass spectrometry (described in greater detail in the Examples below), proteomic arrays, xMAPTM microsphere technology, western blotting, immunohistochemistry, flow cytometry, and detection in body fluid by electrochemical sensor.

For example, mass spectrometry (MS) may be employed. In MS-based methods, a sample (which may be solid, liquid, or gas) is ionized; the ions are separated according to their mass-to-charge ratio, e.g. by magnetic sector, by radio frequencies (RF) quadrupole field, by time of flight (TOF), etc.; the ions are dynamically detected by a mechanism capable of detecting energetic charged particles, and the signal is processed into the spectra of the masses of the particles of that sample. In some instances, tandem mass spectrometry (MS/MS or MS²) may be employed, for example, to determine the sequences of peptides separated by MS. For example, a first mass analyzer isolates one peptide from many entering a mass spectrometer. A second mass analyzer then stabilizes the peptide ions and promotes their fragmentation, e.g. by collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD), surface-induced dissociation (SID), etc. A third mass analyzer then sorts the fragments produced from the peptides. For example, a sample, e.g. a urine sample of the present disclosure, may be applied to an LTQ ion trap mass spectrometer equipped with a Fortis tip mounted nano-electrospray ion source, and the fraction scanned with a mass range of 400-2000 m/z. This first MS scan is followed by two data-dependent scans of the two most abundant ions observed in the first full MS scan. Tandem Miss. can also be done in a single mass analyzer over time, as in a quadrupole ion trap. In some instances, MS is combined with other technologies, e.g. multiple reaction monitoring (MRM) is coupled with stable isotope dilution (SAD) mass spectrometry (MS), which allowed quantitative assays for peptides to be performed with minimum restrictions and the ease of assembling multiple peptide detections in a single measurement. Other methods for detecting peptides in a sample by MS and measuring the abundance of peptides in a sample are well known in the art; see, e.g. the teachings in US 2010/0163721, the full disclosure of which is incorporated herein by reference.

So, for example, in some embodiments, a peptide expression signature, e.g. an NEC-Dx peptide signature, a sepsis-Dx peptide signature, an NEC-M/S peptide signature, is obtained for a patient by obtaining a urine sample from the individual; measuring the abundance of peptide biomarker(s) in the urine sample; and evaluating the abundance of peptide(s) by mass spectrometry. In some embodiments, the patient has already been diagnosed with NEC. Any convenient method for evaluating the abundance of peptides may be employed. For example, the abundance of peptides may be evaluated by summing the amount of each peptide across MS fractions, normalizing to the sum of the amounts of all NEC peptides across all MS fractions to obtain a score for each peptide, and analyzing the scores, e.g. by predictive analysis of microarrays (PAM), to arrive at a single NEC or sepsis peptide signature.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electode.

As another example, flow cytometry may be employed. In flow cytometry-based methods, the quantitative level of polypeptide or peptide fragment of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

For measuring mRNA levels, any convenient method for measuring mRNA levels in a sample may be used, e.g. hybridization-based methods, e.g. northern blotting and in situ hybridization (Parker & Barnes, Methods in Molecular Biology 106:247-283 (1999)), RNAse protection assays (Hod, Biotechniques 13:852-854 (1992)), and PCR-based methods (e.g. reverse transcription PCR(RT-PCR) (Weis et al., Trends in Genetics 8:263-264 (1992)). Alternatively, any convenient method for measuring protein levels in a sample may be used, e.g. antibody-based methods, e.g. immunoassays, e.g., enzyme-linked immunosorbent assays (ELISAs), immunohistochemistry, and flow cytometry (FACS). The starting material may be total RNA, i.e. unfractionated RNA, or poly A+ RNA isolated from a suspension of cells, e.g. a peripheral blood sample. General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR(RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

The resultant data provides information regarding expression for each of the genes that have been probed, wherein the expression information is in terms of whether or not the gene is expressed and, typically, at what level, and wherein the expression data may be both qualitative and quantitative.

Once the expression level of the one or more genes of interest, e.g. NEC-Dx genes, sepsis Dx genes, NEC-M/S genes, has been determined, the measurement(s) may be analyzed in any of a number of ways to obtain an expression signature.

For example, an expression signature may be obtained by analyzing the data to generate an expression profile. As used herein, an expression profile is the normalized expression level of one or more genes of interest in a patient sample. An expression profile may be generated by any of a number of methods known in the art. For example, the expression level of each gene may be log₂ transformed and normalized relative to the expression of a selected housekeeping gene, e.g. ABL1, GAPDH, or PGK1, or relative to the signal across a whole microarray, etc. An expression profile is one example of an expression signature.

As another example, an expression signature may be obtained by analyzed the data to generate an expression score. An expression score is a single metric value that represents the sum of the weighted expression levels of one or more genes of interest in a patient sample. An expression score for a patient sample may be calculated by any of a number of methods known in the art for calculating gene signatures. For example, the expression levels of each of the one or more genes of interest in a patient sample may be log₂ transformed and normalized, e.g. as described above for generating an expression profile. The normalized expression levels for each gene is then weighted by multiplying the normalized level to a weighting factor, or “weight”, to arrive at weighted expression levels for each of the one or more genes, where the weights are defined by a reference dataset, or “training dataset”, e.g. by Principle Component Analysis, Linear discriminant analysis (LDA), Fishers linear discriminant analysis, etc, of a reference dataset. The weighted expression levels are then totaled and in some cases averaged to arrive at a single weighted expression level for the one or more genes analyzed. Any dataset relating to patients having NEC may be used as a reference dataset. For example, the weights may be determined based upon any of the datasets provided in the examples section below. Thus, the NEC-Dx score, sepsis-Dx score, or NEC-M/S score is the first principle component of the NEC-Dx genes, the sepsis-Dx genes, or the NEC-M/S genes, respectively, in a sample as defined by a reference dataset.

As discussed above, expression signatures are obtained by analyzing data on expression levels to arrive at an expression profile or an expression score. This analysis may be readily performed by one of ordinary skill in the art by employing a computer-based system, e.g. using any hardware, software and data storage medium as is known in the art, and employing any algorithms convenient for such analysis.

### Employing an NEC-Dx Expression Signature a Sepsis-Dx Expression Signature, or an NEC-M/S Expression Signature to Evaluate a Subject

The NEC-Dx expression signature, sepsis-Dx expression signature, or NEC-M/S expression signature that is obtained may be employed to diagnose NEC or sepsis, to provide a prognosis to a patient with NEC, or to provide a prediction of the responsiveness of a patient with NEC to a medical therapy. Typically, an expression signature is employed by comparing the expression signature to a reference or control, and using the results of that comparison (a “comparison result”) to determine a diagnosis, prognosis or prediction. The terms “reference” and “control” as used herein mean a standardized gene expression profile, gene signature, or gene score to be used to interpret the NEC-Dx expression signature, Sepsis-Dx expression signature, or NEC-M/S expression signature of a given patient and assign a diagnostic, prognostic, and/or responsiveness class thereto. The reference or control is typically an expression profile or expression score that is obtained from a body fluid or tissue with a known association with a particular phenotype. Additionally, if the expression signature is an expression profile, the reference will typically be an expression profile from a control sample, whereas if the expression signature is an expression score, the reference will typically be the expression score from a control sample.

For example, as disclosed in greater detail in the examples section below, high-risk phenotypes, e.g. significantly different expression of particular panels of genes, are associated with samples from certain patient cohorts, i.e. positive controls. Thus, a positive control reference that may be used when making an NEC diagnosis could be a NEC-Dx signature (e.g. NEC-Dx profile or NEC-Dx score) of a body fluid sample from a patient with NEC; the positive control reference when making a sepsis diagnosis could be a sepsis-Dx signature (e.g. sepsis-Dx profile or sepsis-Dx score) of a body fluid sample from a patient with sepsis; and the positive control reference when providing a prognosis for an individual with NEC or predicting responsiveness of an individual with NEC to medical therapy may be an NEC-M/S signature (e.g. NEC-M/S profile or NEC-M/S score) of a body fluid sample from a patient with either M-class NEC or with S-class NEC.

As another example, low-risk phenotypes e.g. normal expression of particular panels of genes, are associated with sample from unaffected patients, i.e., negative controls. Thus, the negative control reference when making an NEC diagnosis may be a NEC-Dx signature (e.g. NEC-Dx profile or NEC-Dx score) of a body fluid sample from an individual that is not affected with NEC, e.g. a healthy individual, or an individual with sepsis. Likewise, the negative control reference when making a sepsis diagnosis may be a sepsis-Dx signature (e.g. sepsis-Dx profile or sepsis-Dx score) of a body fluid sample from an individual that is not affected with sepsis, e.g. a healthy individual, or an individual with NEC. Similarly, the negative control reference when providing an M-class NEC prognosis may be a NEC-M/S signature (e.g. NEC-M/S profile or score) of a body fluid sample from an NEC individual that is not affected with M-class NEC, e.g. an individual that is affected with S-class NEC or that is not affected with S-class NEC, e.g. an individual that is affected with M-class NEC. In certain embodiments, the obtained expression signature is compared to a single reference/control expression signature to obtain information regarding the phenotype of the tissue being assayed. In yet other embodiments, the obtained expression signature is compared to two or more different reference/control expression signature to obtain more in-depth information regarding the phenotype of the assayed tissue. For example, an expression profile may be compared to both a positive expression profile and a negative expression profile, or an expression score may be compared to both a positive expression score and a negative expression score to obtain confirmed information regarding whether the tissue has the phenotype of interest. As another example, an expression profile or score may be compared to multiple expression profiles or scores, each correlating with a particular diagnosis, prognosis or therapeutic responsiveness, e.g. as might be provided in a report or table that discloses the correlation between particular NEC-Dx, sepsis-Dx, or NEC-M/S signatures and particular disease diagnoses, disease prognoses, or responsiveness to therapy.

As discussed above, an NEC-Dx signature may be employed to make an NEC diagnosis. For example, a patient can be diagnosed as being at high risk for having NEC or as being at low risk for having NEC depending on whether his NEC-Dx signature correlates more closely with the median NEC-Dx signature across a cohort of patients with NEC or whether his signature correlates more closely with the median NEC-Dx signature across a cohort of individuals unaffected by NEC. By “correlates closely”, it is meant is within about 40% of the reference signature, e.g. 40%, 35%, or 30%, in some embodiments within 25%, 20%, or 15%, sometimes within 10%, 8%, 5%, or less. Alternatively, when two or more references are used, e.g. both a reference from a cohort of patient with NEC and a reference from a cohort of unaffected individuals, a patient can be diagnosed as being at high risk for having NEC or as being at low risk for having NEC depending on whether his signature correlates more closely with the median NEC-Dx signature across a cohort of patients with NEC or a cohort of individual unaffected by NEC.

Similarly, a sepsis-Dx signature may be employed to make a sepsis diagnosis. For example, a patient can be diagnosed as being at high risk for having sepsis or as being at low risk for having sepsis depending on whether his sepsis-Dx signature correlates more closely with the median sepsis-Dx signature across a cohort of patients with sepsis or whether his signature correlates more closely with the median sepsis-Dx signature across a cohort of individuals unaffected by NEC. As another example, a patient can be diagnosed as being at high risk for having sepsis or as being at low risk for having sepsis depending on whether his sepsis-Dx signature correlates more closely with the median sepsis-Dx signature across a cohort of patients with sepsis or a cohort of individuals unaffected by sepsis.

In some embodiments, both an NEC diagnosis and a sepsis diagnosis may be made at the same time. In such embodiments, the gene expression levels of one or more of the NEC-Dx genes is measured at the same time that gene expression levels of one or more of the sepsis-Dx genes is measured. In certain embodiments, the NEC-Dx signature and the sepsis-Dx signature may be compared individually, i.e. separately, to one or more references signatures, i.e. the NEC-Dx signature is compared to a reference NEC-Dx signature, and the sepsis-Dx signature is compared to a reference sepsis-Dx signature, and the results of the comparisons are employed to provide a prognosis for the patient. For example, a patient can be diagnosed as being at high risk for having NEC and at low risk for having sepsis or as being at low risk for having NEC and at high risk for having sepsis depending on whether his NEC-Dx and sepsis-Dx signatures correlate more closely with the median NEC-Dx and sepsis-Dx signatures across a cohort of individuals that have NEC, or more closely with the median NEC-Dx and sepsis-Dx signatures across a cohort of individuals that have sepsis. In certain embodiments, the NEC-Dx signature and the sepsis signature are combined to arrive at an NEC/sepsis-Dx signature, the NEC/sepsis-Dx signature is compared to a NEC/sepsis-Dx signature from a reference sample, and the results of the comparisons employed to provide a prognosis for the patient. For example, a patient can be diagnosed as being at high risk for having NEC and at low risk for having sepsis or as being at low risk for having NEC and at high risk for having sepsis depending on whether his combined NEC-Dx signature and sepsis-Dx signature (i.e. his NEC/sepsis-Dx signature) correlates more closely with the median combined NEC-Dx and sepsis-Dx signature across a cohort of patients that have NEC or a cohort of patients that have sepsis.

As also discussed above, an NEC-M/S expression signature may be employed to provide a prognosis to a patient suspected of or diagnosed as having NEC. For example, a patient can be ascribed to high- or low-risk categories, or high-, medium- or low-risk categories for overall survival depending on whether their NEC-M/S signature correlates more closely with the median NEC-M/S signature across a cohort of patients having the M class of the disease or a cohort of patients having the S class of the disease, the overall survival rates of patients with M class NEC or S class NEC being known in the art or readily determined by the ordinarily skilled artisans by, e.g., Kaplan-Meier analysis of individuals with M-class NEC and S-class NEC.

As also discussed above, an NEC-M/S expression signature may be employed to provide a prediction of responsiveness of a patient to a particular therapy, e.g. medical therapy or surgery. These predictive methods can be used to assist patients and physicians in making treatment decisions, e.g. in choosing the most appropriate treatment modalities for any particular patient.

Additionally, the NEC-M/S expression signature may be used on samples collected from patients in a clinical trial and the results of the test used in conjunction with patient outcomes in order to determine whether subgroups of patients are more or less likely to show a response to a new drug than the whole group or other subgroups. Further, such methods can be used to identify from clinical data the subsets of patients who can benefit from therapy. Additionally, a patient is more likely to be included in a clinical trial if the results of the test indicate a higher likelihood that the patient will be responsive to medical treatment, and a patient is less likely to be included in a clinical trial if the results of the test indicate a lower likelihood that the patient will be responsive to medical treatment.

The subject methods can be used alone or in combination with other clinical methods for patient stratification known in the art to provide a diagnosis, a prognosis, or a prediction of responsiveness to therapy. For example, clinical parameters that are known in the art for diagnosing NEC, diagnosing types of NEC, or staging NEC, or for diagnosing or staging sepsis, may also be incorporated into the ordinarily skilled artisan's analysis to arrive at a diagnosis, prognosis, or prediction of responsiveness to therapy with the subject methods.

For example, one common clinically used set of criteria for staging Necrotizing Enterocolitis is Modified Bell's criteria, described in detail in Table 2 below. Other criteria that may be employed for clinical stating include pH value of blood; portal venous gas in x-ray; abdominal ileus in x-ray; the use of a vasopressor prior to diagnosis; abdominal distention; whether cranial ultrasound was done for ivh (intra-ventricular hemorrhage); vasopressor on diagnosis, i.e. the patient is receiving medications that support blood pressure, e.g. inotropes, chronotropes, alpha agonists and the like, e.g. dopamine; ventilation on diagnosis; whether any positive culture of bacteria or fungus was obtained from blood or urine within 5 days of diagnosis; the gestational age of the patient at birth; (and the patient's birth weight. Any criteria as known in the art, e.g. as described above or elsewhere herein, may be used to obtain the subject NEC clinical score for a patient.

TABLE 2
Modified Bell's criteria for staging Necrotizing Enterocolitis.
“NPO” = nothing by mouth

| | | Abdominal | Radiographic | |
|---|---|---|---|---|
| Stage | Systemic signs | signs | signs | Treatment |
| IA | Temperature | Gastric retention, | Normal or | NPO, antibiotics × |
| Suspected | instability, apnea, | abdominal | intestinal | 3 days |
| | bradycardia, | distention, | dilation, mild | |
| | lethargy | emesis, heme- | ileus | |
| | | positive stool | | |
| IB | Same as above | Grossly bloody | Same as above | Same as IA |
| Suspected | | stool | | |
| IIA | Same as above | Same as above, | Intestinal | NPO, antibiotics × |
| Definite, | | plus absent bowel | dilation, ileus, | 7 to 10 days |
| mildly ill | | sounds with or | pneumatosis | |
| | | without | intestinalis | |
| | | abdominal | | |
| | | tenderness | | |
| IIB | Same as above, | Same as above, | Same as IIA, | NPO, antibiotics × |
| Definite, | plus mild | plus absent bowel | plus ascites | 14 days |
| moderately | metabolic | sounds, definite | | |
| ill | acidosis and | tenderness, with | | |
| | thrombocytopenia | or without | | |
| | | abdominal | | |
| | | cellulitis or right | | |
| | | lower quadrant | | |
| | | mass | | |
| IIIA | Same as IIB, | Same as above, | Same as IIA, | NPO, antibiotics × |
| Advanced, | plus hypotension, | plus signs of | plus ascites | 14 days, fluid |
| severely ill, | bradycardia, | peritonitis, | | resuscitation, |
| intact | severe apnea, | marked | | inotropic support, |
| bowel | combined | tenderness, and | | ventilator therapy, |
| | respiratory and | abdominal | | paracentesis |
| | metabolic | distention | | |
| | acidosis, | | | |
| | Disseminated | | | |
| | Intravascular | | | |
| | Coagulation | | | |
| | (DIC), and | | | |
| | neutropenia | | | |
| IIIB | Same as IIIA | Same as IIIA | Same as | Same as IIA, plus |
| Advanced, | | | above, plus | surgery |
| severely ill, | | | pneumo- | |
| perforated | | | peritoneum | |
| bowel | | | | |

A NEC clinical score so obtained may be used in conjunction with the expression signature to provide an NEC diagnosis with greater accuracy, specificity and sensitivity. For example, the NEC-Dx signature and the NEC clinical score are compared to a reference NEC-Dx signature and a reference NEC clinical score, and the results of both comparisons are employed to provide an NEC diagnosis to the patient; or the NEC-M/S signature and the NEC clinical score are compared to a NEC-M/S signature and an NEC clinical score from a reference sample, and the results of both comparisons are employed to provide a sepsis diagnosis to the patient. In some embodiments, the NEC clinical score is used alongside the expression signature of the subject methods. In other embodiments, the NEC clinical score is integrated with the expression score to obtain a single metric value that is representative of both the NEC clinical score and the expression score, i.e. an NEC-gene/clinic score (an “NEC-G/C score”), e.g. an NEC-Dx G/C score, or an NEC-M/S G/C score, where that integrated score is compared to a reference that is an integrated score, at the results of this comparison are employed to provide a prognosis to the patient or to predict the responsiveness of the patient to medical therapy.

As another example, the American College of Chest Physicians and the Society of Critical Care Medicine describes several different levels of sepsis (see Table 3, below). In some embodiments, a sepsis clinical score may be obtained, that sepsis clinical score comprising data on the clinical findings regarding the patient as described in the table. The sepsis clinical score is then used in conjunction with the expression signature to provide a sepsis diagnosis with greater accuracy, specificity and sensitivity. For example, the sepsis-Dx signature and the sepsis clinical score are compared to a sepsis-Dx signature and a sepsis clinical score from a reference sample, and the results of both comparisons are employed to provide an sepsis diagnosis to the patient. In some embodiments, the sepsis clinical score is used alongside the expression signature of the subject methods. In other embodiments, the sepsis clinical score is integrated with the expression score to obtain a single metric value that is representative of both the sepsis clinical score and the expression score, i.e. a sepsis-Dx G/C score, where that integrated score is compared to an integrated score for a reference sample, at the results of this comparison are employed to provide a prognosis to the patient or to predict the responsiveness of the patient to medical therapy.

TABLE 3A
Sepsis levels, as described by the American College of Chest Physicians
and the Society of Critical Care Medicine

* Sepsis.
Defined as a systemic inflammatory response syndrome (SIRS) in response
to a confirmed infectious process. Infection can be suspected or proven (by
culture, stain, or polymerase chain reaction (PCR)), or a clinical syndrome
pathognomonic for infection. Specific evidence for infection includes WBCs
in normally sterile fluid (such as urine or cerebrospinal fluid (CSF), evidence
of a perforated viscus (free air on abdominal x-ray or CT scan, signs of acute
peritonitis), abnormal chest x-ray (CXR) consistent with pneumonia (with
focal opacification), or petechiae, purpura, or purpura fulminans
* Severe sepsis.
Defined as sepsis with organ dysfunction, hypoperfusion, or hypotension.
* Septic shock.
Defined as sepsis with refractory arterial hypotension or hypoperfusion
abnormalities in spite of adequate fluid resuscitation. Signs of systemic
hypoperfusion may be either end-organ dysfunction or serum lactate greater
than 4 mmol/dL. Other signs include oliguria and altered mental status.
Patients are defined as having septic shock if they have sepsis plus
hypotension after aggressive fluid resuscitation (typically upwards of 6
liters or 40 ml/kg of crystalloid).

TABLE 3B
Symptoms indicating potential sepsis in neonates
Body temperature changes
Breathing problems
Diarrhea
Low blood sugar
Reduced movements
Reduced sucking
Seizures
Slow heart rate
Swollen belly area
Vomiting
Yellow skin and whites of the eyes (jaundice)
A heart rate above 160 can also be an indicator of sepsis, this tachycardia
can present up to 24 hours before the onset of other signs.

TABLE 3C
Clinical parameters for sepsis in neonates.

| | |
|---|---|
| 1. | DLC (differential leukocyte count) showing increased numbers of |
| | polymorphs. |
| 2. | DLC (differential leukocyte count)having band cells >20%. |
| 3. | increased haptoglobins. |
| 4. | micro ESR (Erythrocyte Sedimentation Rate) titer > 55 mm. |
| 5. | gastric aspirate showing >5 polymorphs per high power field. |
| 6. | newborn CSF (Cerebrospinal fluid) screen: showing increased cells |
| | and proteins. |
| 7. | suggestive history of chorioamnionitis, PROM (Premature rupture |
| | of membranes), etc. |

Culturing for microorganisms from a sample of CSF, blood or urine, is the gold standard test for definitive diagnosis of neonatal sepsis. This can give false negatives due to the low sensitivity of culture methods and because of concomitant antibiotic therapy. Lumbar punctures should be done when possible as 10-15% presenting with sepsis also have meningitis, which warrants an antibiotic with a high CSF penetration.

In some embodiments, providing an evaluation of a subject with suspected or confirmed NEC or sepsis, i.e., providing an NEC-Dx signature, a sepsis Dx signature, an NEC-M/S signature, a diagnosis of NEC or of sepsis, a prognosis for a patient with NEC, or a prediction of responsiveness of a patient with NEC to therapy, includes generating a written report that includes the artisan's assessment of the subject's current state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document that includes report elements that provide information of interest relating to a diagnosis assessment, a prognosis assessment, and/or a treatment assessment and its results. A subject report can be completely or partially electronically generated. A subject report typically includes at least a NEC-Dx signature, a sepsis Dx signature, or an NEC-M/S signature, and/or at least a diagnosis assessment, i.e. a diagnosis as to whether a subject has a high likelihood of having NEC or sepsis; or a prognosis assessment, i.e. a prediction of the likelihood that a patient with NEC will have an NEC-attributable death; or a treatment assessment, i.e. a prediction as to the likelihood that an NEC patient will have a particular clinical response to treatment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include i) the gene expression levels of one or more NEC-Dx genes, sepsis-Dx genes, NEC-M/S genes, ii) the gene expression profiles for one or more NEC-Dx, sepsis-Dx, NEC-M/S genes, and/or iii) an NEC-Dx, sepsis-Dx, or NEC-M/S signature, b) reference values employed, if any; 6) other features.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, urine, saliva), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will have an NEC-attributable death or progression. The interpretive report can include, for example, results of the gene expression analysis, methods used to calculate the NEC-Dx, sepsis-Dx, NEC-M/S signature, and interpretation, i.e. prognosis. The assessment portion of the report can optionally also include a Recommendation(s). For example, where the results indicate that the subject has NEC, the recommendation can include a recommendation that broad-spectrum antibiotics be provided and that no nutrition be provided by mouth.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of assaying gene expression levels, where such reagents may include RNA or protein purification reagents, antibodies to NEC-Dx polypeptides or peptides thereof, sepsis-Dx polypeptides or peptides thereof, and/or NEC-M/S polypeptides or peptides thereof, (e.g., immobilized on a substrate), nucleic acid primers specific for NEC-Dx genes, sepsis-Dx genes, and/or NEC-M/S genes, arrays of nucleic acid probes, signal producing system reagents, etc., depending on the particular detection protocol to be performed.

For example, reagents may include protein affinity reagents or oligonucleotides that are specific for one or more genes selected from the group consisting of CD14, SAP1, PEDF, Q6ZUQ4, OBFC2B, COL11A2, NBEAL2, GRASP, HUWE1, COL1A2, HOXD3, DSG4, KRTAP5-11, Y1020, FGA, UMOD, CTAPIII, SAA1, B2M, TTR, OSTP, APOA4, C08G, ANGT, FIBA, PROF1, PLSL, LMAN2, ftsY, PROC, MAP1B, CSN5, A2ML1, CST3, RET4, and VASN. Particular combinations of affinity reagents or oligonucleotides of interest include affinity reagents or oligonucleotides specific for CST3, PEDF, and RET4/RBP4; affinity reagents or oligonucleotides specific for A2ML1, CST3, FGA, and VASN; affinity reagents or oligonucleotides specific for CST3, PEDF, and VASN; affinity reagents or oligonucleotides specific for A2ML1, CD14, CST3, PEDF, RET4, and VASN; and affinity reagents or oligonucleotides specific for one or more of the FGA peptides selected from the group consisting of DEAGSEADHEGTHSTKR, DEAGSEADHEGTHSTKRG, and DEAGSEADHEGTHSTKR-GHAKSRPV.

Other examples of reagents include arrays that comprise probes, e.g. arrays of antibodies or arrays of oligonucleotides; or other reagents that may be used to detect the expression of NEC-Dx genes, sepsis-Dx genes, and/or NEC-M/S genes.

The subject kits may also comprise one or more expression signature references, e.g. a reference for an NEC-Dx signature, a reference for a sepsis-Dx signature, and/or a reference for an NEC-M/S signature, for use in employing the expression signature obtained from a patient sample. For example, the reference may be a sample of a known phenotype, e.g. an unaffected individual, or an affected individual, e.g. from a particular risk group that can be assayed alongside the patient sample, or the reference may be a report of disease diagnosis, disease prognosis, or responsiveness to therapy that is known to correlate with one or more of the subject expression signatures. As another example, one type of reagent that is specifically tailored for generating peptide signatures, e.g. a NEC-Dx peptide signature, sepsis-Dx peptide signature, or NEC-M/S peptide signature, peptide representations, is a collection of isotope labeled- and unlabeled-peptides that may be used for calibration and as internal references, e.g. in spectrometry methods, e.g. mass spectrometry (MS)-based methods.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Necrotizing enterocolitis (NEC) is a major cause of overall neonatal morbidity and mortality. Disease outcome for infants with NEC is largely determined by the degree of clinical progression. Generally, half of affected infants recover with medical therapy alone (NEC M=medical class) and 30-50% develop progressive disease requiring surgery or resulting in death (NEC S=surgical class). Most of the disease associated morbidity, and nearly all of the mortality, occurs in the cohort with progressive disease requiring surgery. Previous attempts to identify clinical parameters that could reliably identify infants with NEC most likely to progress to severe disease have been unsuccessful. We hypothesized that an integrative analysis of clinical parameters along with protein biomarkers would result in a predictive algorithm of NEC progression. A multivariate analysis of patients (NEC 43 M and 26 S) using the standard NEC classification scheme of Bell failed to differentiate NEC outcomes. A novel panel of eleven clinical parameters, selected by Mann Whitney U test, (NEC 43 M and 26 S subjects) as a biomarker panel did stratify NEC subjects into low, intermediate and high-risk groups for progression. Molecular profiling of the urine peptidome (NEC 17 M and 11 S subjects) and plasma proteome (NEC 60 M and 30 S subjects) identified separate candidate biomarker panels of 36 urine peptides and 30 plasma proteins as biomarkers for progressive NEC. Complete clinical and molecular records were available for 13 NEC M and 11 NEC S patients affording detailed comparative analyses of the statistical performance of the clinical (P=0.64), urine (P=9.5×10⁻⁴), and plasma panels (P=1.3×10⁻³) for NEC progression classification. Integrative analyses combining the clinical parameters, urine peptides and plasma proteins improved the NEC progression predictive performance (P value of 5.2×10⁻⁴), leading to an optimal biomarker panel (15 urine peptides and 3 plasma proteins) that discriminates NEC M and S class with high sensitivity and specificity (P value of 4.0×10⁻⁷ and ROC AUC 0.99). We conclude that ensemble data mining methods utilizing clinical and molecular based classifiers produces effective predictive integrated algorithm for NEC progression.

### Materials and Methods

Clinical Data Collection.

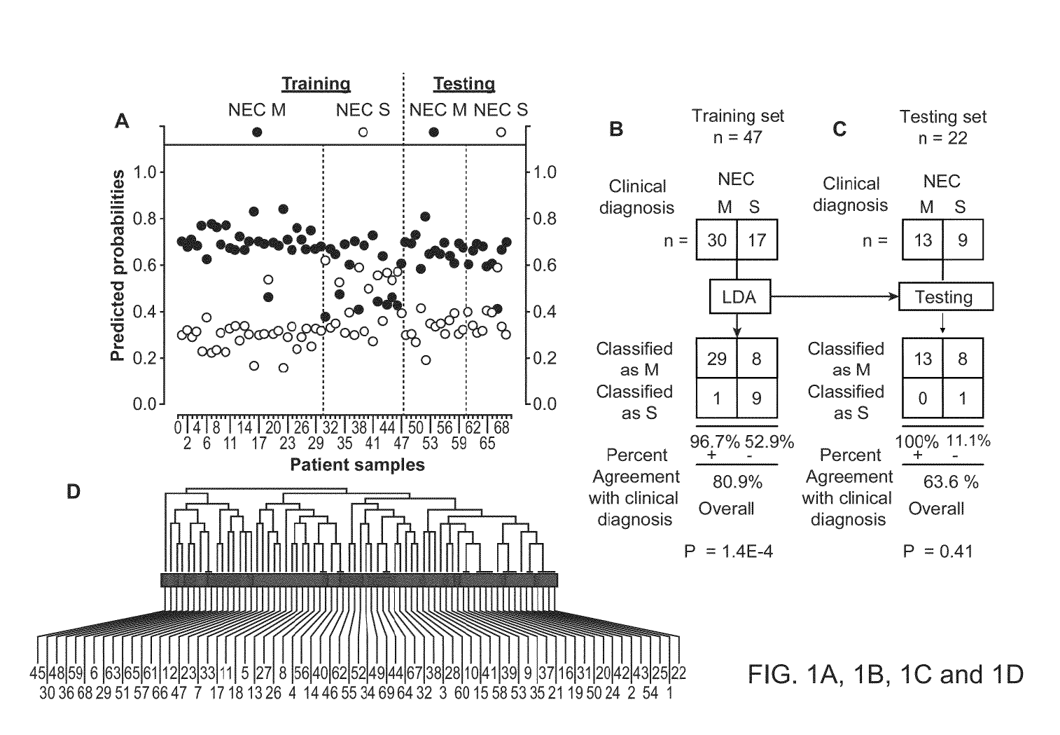

All 50 clinical and demographic parameters, summarized in Table 3, relevant to the initial diagnosis of NEC were extracted from an observational, prospective cohort study conducted by the NEC consortium consisting of the following institutions: Texas Children's Hospital, Houston; Lucile Packard Children's Hospital, Stanford; Johns Hopkins Children's Hospital, Baltimore; The Children's Hospital of Philadelphia; and Yale-New Haven Children's Hospital. In this study, infants who met at least one criterion from each of the three Modified Bell's criteria categories including historical factors, physical findings and radiological findings, were identified as suspicious for or diagnostic of NEC and became eligible for the study. Historical factors include feeding intolerance (defined as vomiting two or more feedings within 24 hours or any vomitus containing bile, or the presence of gastric residuals of volume greater than 6 cc/kg or any aspirate containing bile), apneic/bradycardic episodes, oxygen desaturation episodes, guaiac positive, or bloody stools. Physical findings include abdominal distention, capillary refill time>2 sec, abdominal wall discoloration, or abdominal tenderness. Radiological findings include pneumatosis intestinalis, portal venous gas, Ileus, dilated bowel, pneumoperitoneum, air/fluid levels, thickened bowel walls, ascites or peritoneal fluid, or free intraperitoneal air. The following variables were not included in the consortium NEC database but are part of the Modified Bells Criteria: temperature instability, absent bowel sounds, hypotension, abdominal cellulitis, and right lower quadrant mass. A total of 15 clinical parameters were utilized, upon data availability, as the NEC modified staging criteria and are detailed in Table 4 and FIG. 1A.

TABLE 4
Clinical parameters for NEC progression analysis

| | | Bells Criteria | | |
|---|---|---|---|---|
| | | Used in initial | | 11 Clinical |
| | | screening for | Modified | Parameters |
| Clinical | Description of the | eligibility in the | Bells | used in NEC |
| variables | clinical variables | NEC database | Criteria | stratification |
| patgen | Gender; 1 = male 2 = female | | | |
| datebirth | Date of birth | | | |
| gestage | gestational age | | | X |
| birthwt | birthweight in grams | | | X |
| prodef | date in which patient met | | | |
| | definition of NEC | | | |
| TIMEBTWN | time between enrollment | | | |
| | and specimen | | | |
| | collection | | | |
| first_endpt | First endpoint: S = surg. | | | |
| | T = transp. E = end fu | | | |
| | X = non-nec F\|R = full | | | |
| | feeds D = death | | | |
| severedate | Date of 1st surgery or death | | | |
| surg1day | Day between enrollment and | | | |
| | 1st surgery | | | |
| surg2day | Day between enrollment and | | | |
| | 2nd surgery | | | |
| surgdatel | Date of 1st surgery | | | |
| surgdate2 | Date of 2nd surgery | | | |
| deathdt | Date of death | | | |
| bloodstool | gross blood in stool; 1 = yes | X | X | |
| | 0 = no | | | |
| abddistend | abdominal distention | X | X | X |
| caprefill | capilary refill time >2 | X | X | |
| | seconds | | | |
| abdcolor | abdominal discoloration | X | X | |
| abdpain | abdominal tenderness | X | X | |
| pintestin | pneumatosis intestinalis in | X | X | |
| | x-ray | | | |
| portvenous | portal venous gas in x-ray | X | X | X |
| abdileus | abdominal ileus in x-ray | X | X | X |
| dilatebowel | dilated bowel in x-ray | X | X | |
| air | air or fluid in x-ray | X | X | |
| pnemo | pneumoperitoneum in x-ray | X | X | |
| thickbowel | thickened bowel in x-ray | X | X | |
| ascites | ascites or peritoneal fluid | X | X | |
| | in x-ray | | | |
| freeipair | free intraperitoneal air | X | X | |
| venton | ventilation on dx | | | X |
| ventpri | No of days on ventilation | | | |
| | prior to dx | | | |
| ventever | Ever on ventilation if no | | | |
| | ventilation on dx? | | | |
| cpapon | CPAP on dx | | | |
| cpappri | No of days on CPAP prior | | | |
| | to dx | | | |
| cpapever | Ever on CPAP if no CPAP | | | |
| | on dx? | | | |
| vasspri | ever on vassopressor prior | | | X |
| | to dx? | | | |
| vasson | vassopressor on dx | | | X |
| antion | antiobiotics on dx? | | | |
| cranult | cranial ultrasound done for | | | X |
| | ivh? | | | |
| entnutrec | enteral nutrient on dx? | | | |
| culturefive | any pos culture within 5 | | | X |
| | days of dx | | | |
| cultsix | any pos culture 6-14 days | | | |
| | before dx | | | |
| wbccell | wbc | | X | |
| neutcount | neutrophil count | | | |
| neutperc | neutroperc | | | |
| bandscount | bands count | | | |
| bandsperc | bands percentage | | | |
| platcount | platelet counts | | X | |
| hemocrit | hematocrit | | | |
| reacpro | CRP | | | |
| phval | pH values | | X | X |
| phsite | site where blood was | | | |
| | collected for pH | | | |

Three cohort sets of patient data were analyzed: (1) clinical findings on 69 patients including Bell's NEC staging criteria (Table 5); (2) urine peptidomes on 34 individual patients (Table 6); and (3) plasma proteomes on 90 individual patients (Table 7).

TABLE 5
Demographics between Non-Progressive vs Progressive NEC
patients in the Clinical Assays. Mann Whitney test for continuous
variables and Fischer Exact test for dichotomous variables.
[ ] represents 95% confidence interval. ( ) represents percentages.

| | Non-Progressive | | Progressive | | p-value |
|---|---|---|---|---|---|
| | n = 43 (62.3%) | | n = 26 (37.7%) | | |
| Male | 19 | (44.2%) | 18 | (69.2%) | 0.051 |
| Gestational Age (week) | 29.8 | | 28.4 | | 0.122 |
| | [28.0-30.9] | | [27.0-28.9] | | |
| Birth Weight (gm) | 1343.4 | | 1164.3 | | 0.220 |
| | [1130.9-1556.0] | | [932.1-1396.6] | | |
| Race | | | | | 0.315 |
| Caucasian | 21 | (48.8%) | 9 | (34.6%) | |
| Black | 15 | (34.9%) | 11 | (42.3%) | |
| Asian | 3 | (7.0%) | 0 | (0%) | |
| Native Hawaiian/Pacific | 0 | (0%) | 1 | (3.9%) | |
| Islander | | | | | |
| American Indian/Alaskan | 0 | (0%) | 0 | (0%) | |
| Native | | | | | |
| Latino or Hispanic | 9 | (20.3%) | 6 | (23.1%) | 1.000 |

TABLE 6
Demographics Among Non-Progressive and Progressive NEC
patients in the Urine Assays. General Linear Model & ANOVA
with Scheffe adjustment for continuous variables and
Fischer Exact test for dichotomous variables. [ ] represents
95% confidence interval. ( ) represents percentage.

| | Non-Progressive | | Progressive | | |
|---|---|---|---|---|---|
| | n = 17 (50.0%) | | n = 11 (32.4%) | | p-value |
| Male | 7 | (41.2%) | 10 | (90.9%) | 0.025 |
| Gestational Age | 28.9 | | 28.0 | | 0.236 |
| (week) | [27.3-30.6] | | [25.9-30.1] | | |
| Birth Weight (gm) | 1230.5 | | 1167.9 | | 0.609 |
| | [917.3-1543.7] | | [778.6-1557.3] | | |
| Race | | | | | 0.138 |
| Caucasian | 12 | (70.6%) | 4 | (36.3%) | |
| Black | 3 | (17.7%) | 5 | (45.5%) | |
| Asian | 2 | (11.7%) | 0 | (0%) | |
| Native | 0 | (0%) | 0 | (0%) | |
| Hawaiian/Pacific | | | | | |
| Islander | | | | | |
| American | 0 | (0%) | 0 | (0%) | |
| Indian/Alaskan | | | | | |
| Native | | | | | |
| Latino or Hispanic | 2 | (11.8%) | 3 | (27.3%) | 0.449 |

TABLE 7
Demographics between Non-Progressive vs Progressive NEC
patients in the Plasma Assay. Mann Whitney test for
continuous variables and Fischer Exact test for dichotomous
variables. [ ] represents standard deviations.
( ) represents percentage.

| | Non-Progressive | | Progressive | | |
|---|---|---|---|---|---|
| | n = 60 (66.7%) | | n = 30 (33.7%) | | p-value |
| Male | 25 | (43.1%) | 20 | (69.0%) | 0.026 |
| Gestational Age | 30.2 | | 28.2 | | 0.031 |
| (week) | [29.1-31.3] | | [26.8-29.5] | | |
| Birth Weight (gm) | 1453.8 | | 1128.4 | | 0.054 |
| | [1245.0-1662.7] | | [916.2-1340.6] | | |
| Race | | | | | 0.140 |
| Caucasian | 32 | (55.2%) | 11 | (37.9%) | |
| Black | 17 | (29.3%) | 12 | (41.4%) | |
| Asian | 4 | (6.9%) | 0 | (0%) | |
| Native | 0 | (0%) | 1 | (3.5%) | |
| Hawaiian/Pacific | | | | | |
| Islander | | | | | |
| American | 0 | (0%) | 0 | (0%) | |
| Indian/Alaskan | | | | | |
| Native | | | | | |
| Latino or Hispanic | 11 | (19.0%) | 6 | (20.7%) | 1.000 |

Patient Demographics Analysis.

Once enrolled, epidemiologic data were abstracted from the patient's chart as previously described (3) until one of several end-points was reached. Proportion and its confidence interval were employed to identify possible outliers in the non-progressive and progressive NEC patients. Fisher's exact test, Student T test and Mann Whitney U test were performed to examine the distribution of each demographic variable between non-progressive and progressive NEC patients. A general linear model with ANOVA was conducted to compare each demographic variable among the non-progressive and progressive. Scheffe adjustment was added to correct the p-values for multiple pair-wise comparisons. All analyses on the demographic variables were executed using SAS statistical software version 9.1.3.

Urine Collection, Storage and Processing.

Intra day urine samples (0.5 mL˜1 mL) were collected in sterile tubes and held at 4° C. for up to 8 h before centrifugation (2,000 g×20 min at room temperature) and freezing of the supernatant at −70° C. The details of urine processing, preparation of peptides, extraction and fractionation are reported elsewhere (13)

Urine Peptidomic MS Data Analysis.

The ABI 4700 oracle database MS spectra were exported as raw data points via ABI 4700 Explorer software ver 2.0 for subsequent data analyses. The m/z ranges were from 800 to 4000 with peak density of maximum 30 peaks per 200 Da, minimal S/N ratio of 5, minimal area of 10, minimal intensity of 150, and 200 maximum peaks per spot. An informatics platform was previously developed which contains an integrated set of algorithms, statistical methods, and computer applications, to allow for MS data processing and statistical analysis of liquid chromatography-mass spectrometry (LCMS) based urine peptide profiling. The MS peaks are located in the raw spectra of the matrix-assisted laser desorption/ionization (MALDI) data by an algorithm that identifies sites (mass-to-charge ratio, m/z values) whose intensity is higher than the estimated average background and the ˜100 surrounding sites, with peak widths ˜0.5% of the corresponding m/z value. To align peaks from the set of spectra of the assayed samples, linkage hierarchical clustering was applied to the collection of all peaks from the individual spectra. The clustering, computed on a 24 node LINUX cluster, is two dimensional, using both the distance along the m/z axis and the HPLC fractionation time, with the concept that tight clusters represent the same biological peak that has been slightly shifted in different spectra. The centroid (mean position) of each cluster was then extracted to represent the “consensus” position as the peak index (bin) across all spectra.

MS/MS Analysis for Peptide Biomarkers.

The approach of ion mapping was used to obtain protein identification. In ion mapping, biomarker candidate mass spectra (MS) peaks are selected on the basis of discriminant analysis and then targeted for MS/MS sequencing analysis. Extensive MALDI-TOF/TOF and LTQ Orbitrap MS/MS analyses coupled with database searches were then performed to sequence and identify these peptide biomarkers. The identity of a subset of peptides detected was determined by searching MS/MS spectra against the Swiss-Prot database (Jun. 10, 2008) restricted to human entries (15,720 sequences) using the Mascot (version 1.9.05) search engine. Searches were restricted to 50 and 100 ppm for parent and fragment ions, respectively. No enzyme restriction was selected. Since we were focusing on the naturally occurring peptides, hits were considered significant when they were above the statistical significant threshold (as returned by Mascot). Selected MS/MS spectra were also searched by SEQUEST (BioWorks™ rev.3.3.1 SP1) against the International Protein Index (IPI) human database version 3.5.7 restricted to human entries (76,541 sequences). mMASS, an open source mass spectrometry tool (http://mmass.biographics.cz/), was used for manual review of the protein identification and MS/MS ion pattern analysis for additional validation. Different fragmentation techniques were used for the validation of a peptide sequence, as well as for the detection, localization and characterization of post-translational modifications.

Pathway Analysis.

The PANTHER (Protein ANalysis THrough Evolutionary Relationships) Classification System (20) is a unique resource that classifies proteins by their functions and molecular pathways, using published scientific experimental evidence and evolutionary relationships. The protein IDs of the protein precursors of the urine peptide biomarker candidates were uploaded to PANTHER 7.0 (http://www.pantherdb.org/) to explore the molecular pathways these biomarkers might involve.

SELDI-TOF MS, Analysis and Feature Extraction.

Aliquots of plasma were thawed, denatured, and fractionated on an anion exchange column using the Expression Difference Mapping kit from Ciphergen Biosystems in conjunction with Beckman Biomek 2000 robot. Each plasma sample was processed in duplicate; including controls. For fractionation, 20 mL of each plasma was denatured with 30 mL 9 M urea, diluted to 1 M urea at pH 9, and applied to Q ceramic HyperD F ion-exchange beads (strong anion exchanger). The pass-through and a pH 9 wash were combined as fraction 1 by filtration of the beads in a 96-well vacuum filtration plate (Millipore, Bedford, Mass., USA). Fractions 2 (pH 7), 3 (pH 5), 4 (pH 4), 5 (pH 3), and 6 (organic elution) were similarly collected. All fractions had a total volume of 200 mL per sample and were stored at −80° C. until further processing. For SELDI analysis, fractions were thawed and 10 mL aliquots of each sample were diluted ten-fold in binding buffer appropriate for the CM10 (weak cation exchanger, 0.1 M acetate pH 4.0) and HSO(RP, water:ACN:TFA 90:10:0.1), Ciphergen SELDI surfaces. Each surface was prepared according to the manufacturer's instructions and then incubated with each appropriately diluted sample. After incubation, each surface was washed successively with binding buffer and water. After brief air-drying, 1 mL of saturated sinapinic acid was added twice to each spot. Mass spectra of spotted samples were obtained using Ciphergen PBSIIc mass spectrometer. The detector voltage was set to 2900 V, laser intensity 170, and detector sensitivity. Data collection was optimized for m/z 3000-30 000, and the digitizer frequency was 250 MHz. Spectra were collected by Ciphergen ProteinChip software 3.2 and exported to CDM and feature extraction was performed using the software “Simultaneous Spectrum Analysis” (SSA).

Statistical Analyses.

Hypothesis testing used Student t test and Mann-Whitney U test, and global and local FDR to correct for multiple hypothesis testing issues. Nearest shrunken centroid (NSC) based feature selection, including permutation based FDR analysis, was performed using R PAM package. Unsupervised heatmap analyses were performed using R stats package. Binary class clustering results were grouped into modified 2×2 contingency tables, which were used to calculate the proportion of the clustering results that agreed with clinical diagnosis and the statistical significance by the Fisher's exact test. Supervised linear discriminant analysis for binary (NEC M and S) classifications, using R MASS package, led to the predictive linear discriminant analysis models. The predictive performance of each linear discriminant analysis model was evaluated by ROC curve analysis. The class prediction results were grouped in modified 2×2 contingency tables and the statistical significance of the extent of agreement with clinical diagnosis was assessed by Fisher's exact test.

Predictive probabilities from the linear discriminant model (LDA) of NEC clinical parameter panel (11 clinical parameters) were transformed into scores.

NEC clinical score:

X=scale(log(Clinical model LDA P value×100))×10

Scale is a generic R function whose default method centers and/or scales the columns of a numeric matrix. The scoring metrics enable the clinical parameter based classifier to be interpreted on a scale, rather than a strict binary discrimination. This increases the flexibility and the collective use of each of the panel components. NEC subjects were sorted by the corresponding NEC clinical scores (from smallest to largest) and stratified. For each patient, the percentage of NEC subjects with equal or lower score was plotted against this subject's clinical score. Visual inspection of the NEC score percentile versus the NEC clinical score plot separated the patients into low, intermediate and high-risk groups. Each group's risk of NEC progression was quantified as the proportion of NEC S class diagnoses among the group's patients.

### Results

Patient Demographics and Characteristics.

In this study, a systematic approach was taken to discover biomarkers of NEC progression by examining three cohort sets of patient data: (1) clinical findings on 69 patients including Bell's NEC staging criteria; (2) urine peptidomes on 34 individual patients; (3) plasma proteomes on 90 individual patients. Among these different data sets, 24 patients (NEC 13 M and 11 S) had complete data for clinical findings and molecular profiles for both urine peptidome and plasma proteomes. Each cohort's sample demographics are described in Table 4, 5, and 6 of the methods section, respectively. Statistically significant differences (P value<0.01) in patient demographics were found for gestational age and gender, each of which has been cited previously.

Bell's NEC Staging System Cannot be Used for NEC Progression Risk Prediction.

The NEC staging system according to Bell (Bell's Criteria) is commonly used to diagnose and more generally characterize the severity of NEC (16). Utilizing the clinical parameters that comprise Bell's modified criteria (Bell's modified criteria: Feeding intolerance, Apneic/bradycardic episode, Oxygen desaturation episoe, Grossy bloody stools, Abdominal distention, Abdominal tenderness, Pneumatosis intestinalis, Portal venous gas, Lleus, Dilated bowel, Pneumoperitoneum, Air/Fluid levels, Thickened bowel, Ascites or peritoneal fluid, Free intraperitoneal air; clinical parameters detailed in Table 4 of the methods section), linear discriminant analysis was performed on a training data set from NEC M (n=30) and S (n=17) samples. The resultant LDA model was then tested on a new data set comprised of NEC M (n=13) and S (n=9) samples. The predicted probabilities for the progression of NEC for both the training (left) and testing data (right) were plotted (FIG. 1A) for each of the patients. In FIGS. 1B and 1C (FIG. 1B training and FIG. 1C testing), samples are partitioned by the true class (upper) and predicted class (lower). The 2×2 contingency tables summarize the classification results for NEC progression. In the training set, an overall 80.9% agreement with the clinical outcome is realized using the LDA model (29/30 NEC-M and 9/17 NEC-S; P value of 1.4×10−4), however only 52.9% of the NEC S subjects were classified correctly. Using the LDA model to analyze a new independent dataset for testing yields a mere 11.1% (1 of 9) correctly classified as progressive NEC, and an overall 63.6% agreement (p=0.41) with the clinical diagnosis (FIG. 1D) when both medical and surgical outcomes are considered together. Unsupervised clustering of all 69 samples revealed no obvious pattern, supporting the findings from the supervised learning that Bell's NEC staging criteria are inadequate for predicting the risk of NEC progression.

11 Clinical-Parameter Based Classifier was Developed for NEC Patient Stratifications.

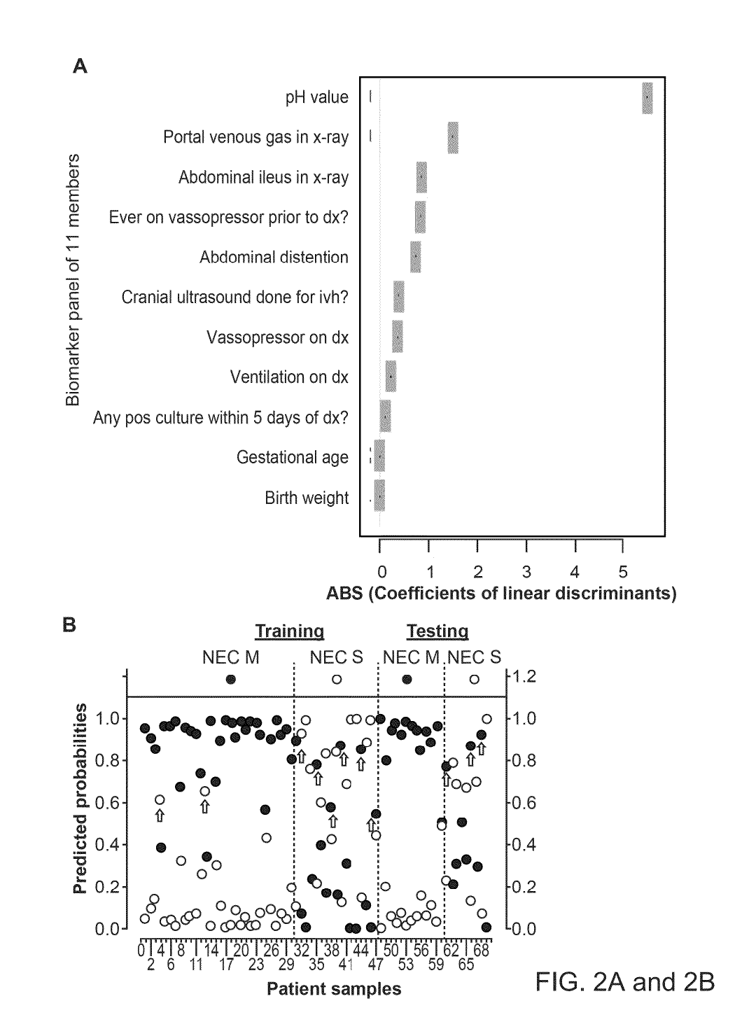

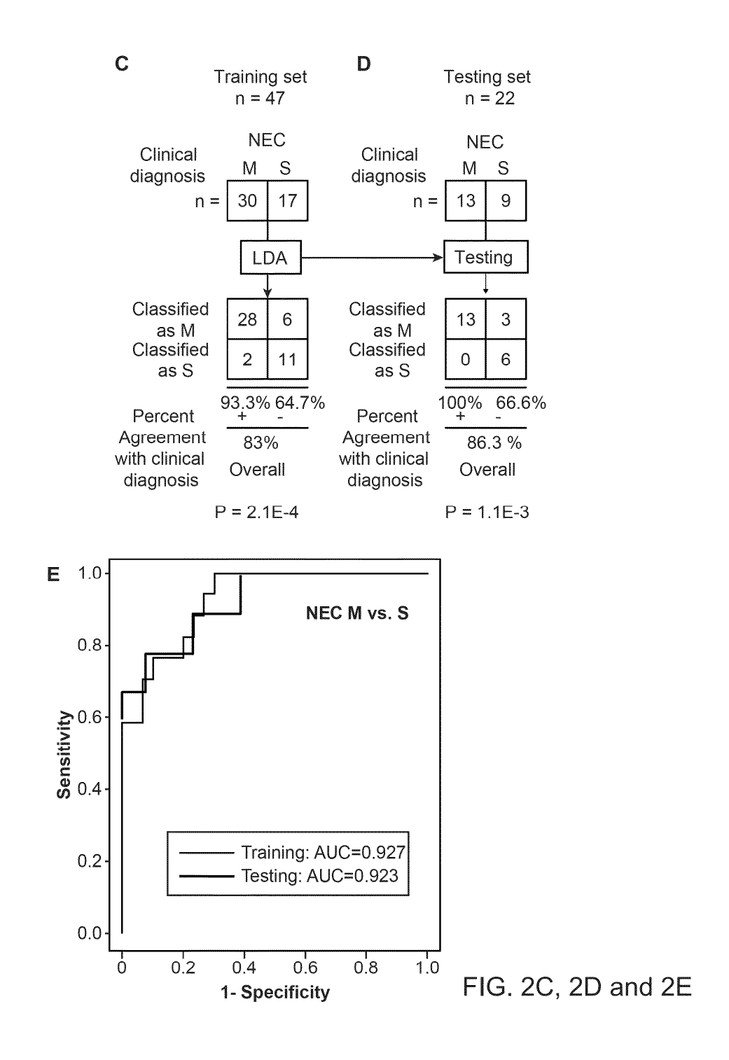

Detailed clinical data for 50 distinct clinical parameters (Table 3) were collected and Mann Whitney U test was used to analyze NEC M (n=43) and S (n=26) patient groups. Eleven clinical parameters (pH value of blood), Portal venous gas in x-ray, Abdominal ileus in x-ray, use of vassopressor prior to diagnosis, Abdominal distention, Cranial ultrasound done for ivh, Vassopressor on diagnosis, Ventilation on diagnosis, Any positive culture within 5 days of diagnosis, Gestational age, Birth weight; Mann Whitney U test P value<0.1) were selected for subsequent LDA modeling, and the corresponding absolute values (ABS) of the first linear discriminant (LD1) from the LDA were plotted. The clinical parameters of pH, portal venous gas on x-ray, abdominal ileus by x-ray, use of vasopressor medications prior to diagnosis, and abdominal distention were found to be the most distinguishing clinical parameters between NEC classifications for M and S subjects. The use of the 11 clinical parameter panel on a training (NEC 30 M and 17 S) and test set (NEC 13 M and 9 S) revealed good separation between the highest and next highest probability for the classification (FIG. 2A). Overall, 28 of the 30 NEC M and 11 of the 17 NEC S in the training set, and 13 of the 13 NEC M and 6 of the 9 NEC S in the testing set were classified correctly. Overall, the 11-clinical-parameter panel classified the training and test sets with a performance P value of 2.1×10⁻⁴ (AUC of ROC: 0.927) and 1.1×10⁻³ (AUC of ROC: 0.923) respectively. However, the NEC S prediction rates were sub-optimal with only 64.7% and 66.6% agreeable with the clinical diagnosis.

Urine 36-Peptide Panel Effectively Classified NEC M and S Patients.

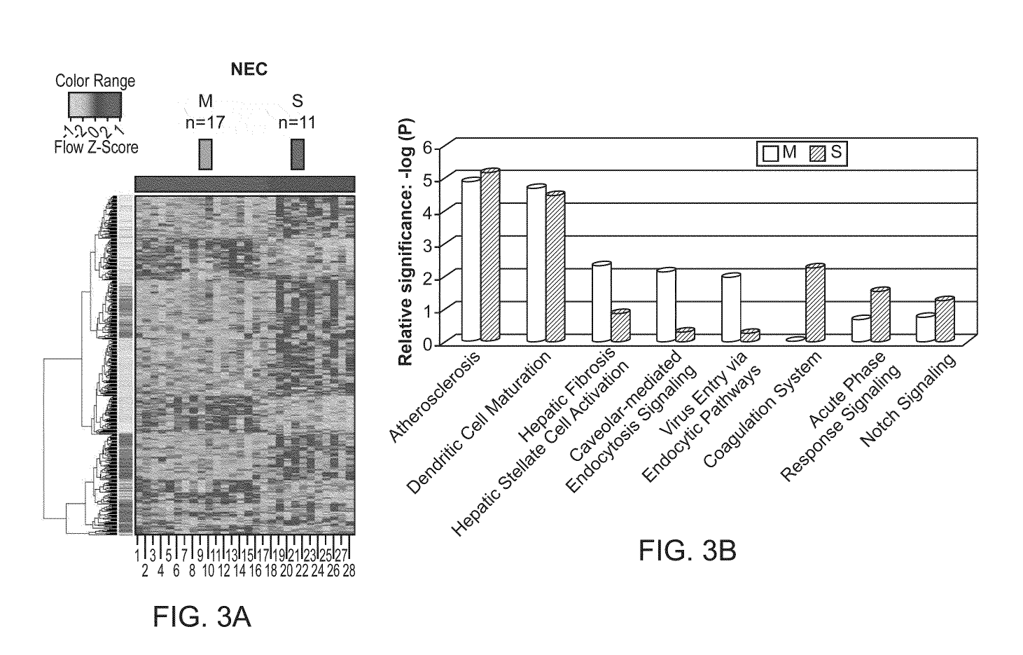

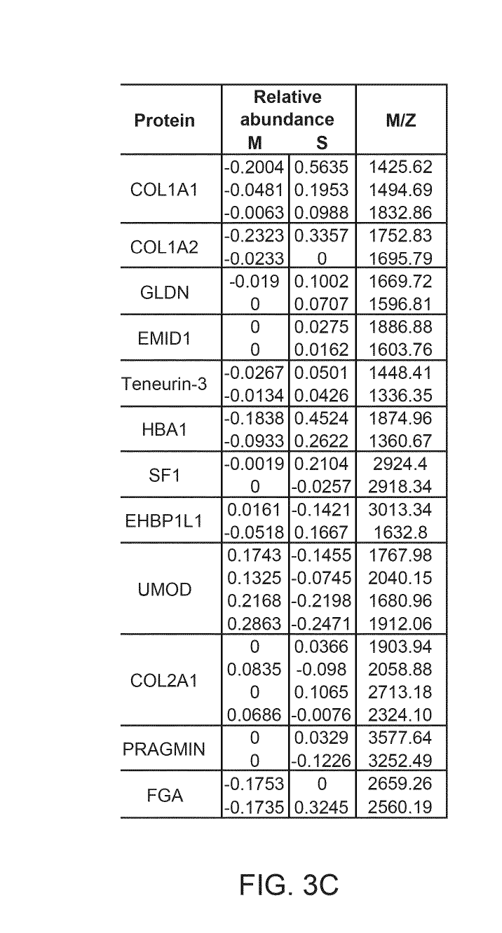

MALDI-TOF mass spectrometry (MS) based urine peptidomic analysis resulted 120 HPLC fractions for each sample, resolving a total of 17,173 peptide peaks defined by distinct m/z and HPLC fractions in the 900- to 4000-Da range. All the features were ranked by a nearest shrunken centroid (NSC) algorithm (26) in order to differentiate NEC M (n=17) and S (n=17) groups. For the NEC-S class, 6 patient samples were obtained following surgery, the reminder (n=11) were obtained at the time of diagnosis, same as the samples for the NEC-M class patients. Next, the top 1000 peaks were subject to extensive MSMS protein identification yielding 473 distinct peptides. Unsupervised cluster and pathway analyses of these identified urine peptides were performed for the NEC M (n=17), S (n=11) and post surgical (Post S, n=6) subjects. Manual examination of the heat map display of unsupervised clustering revealed that the 473 urine peptides can be largely grouped into 2 bins: (I) peptides up regulated in NEC S, then down in NEC M; (II) upregulated in NEC M, down in NEC S samples. Data mining software (Ingenuity Systems, www.ingenuity.com, CA) was used to analyze these differential urine peptides' parent proteins and to identify significant gene ontology groups and relevant signaling pathways. As shown in FIG. 3B, the analysis of significance (−log(P)) of the canonical pathways could largely group them into 3 bins: (1) similarly significant in NEC M and S: atherosclerosis, dendritic cell maturation, notch signaling; (2) more significant in NEC M than S: hepatic fibrosis/hepatic stellate cell activation, caveolar-mediated endocytosis signaling, virus entry via endocytic pathways; (3) more significant in NEC S than M: coagulation system, acute phase response signaling.

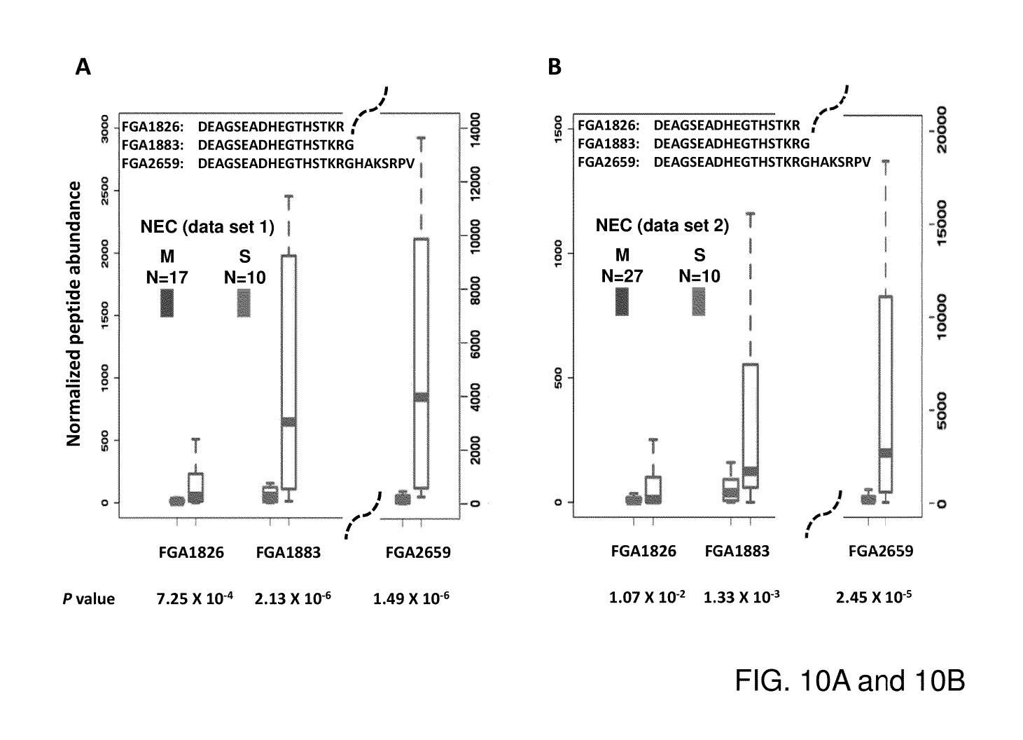

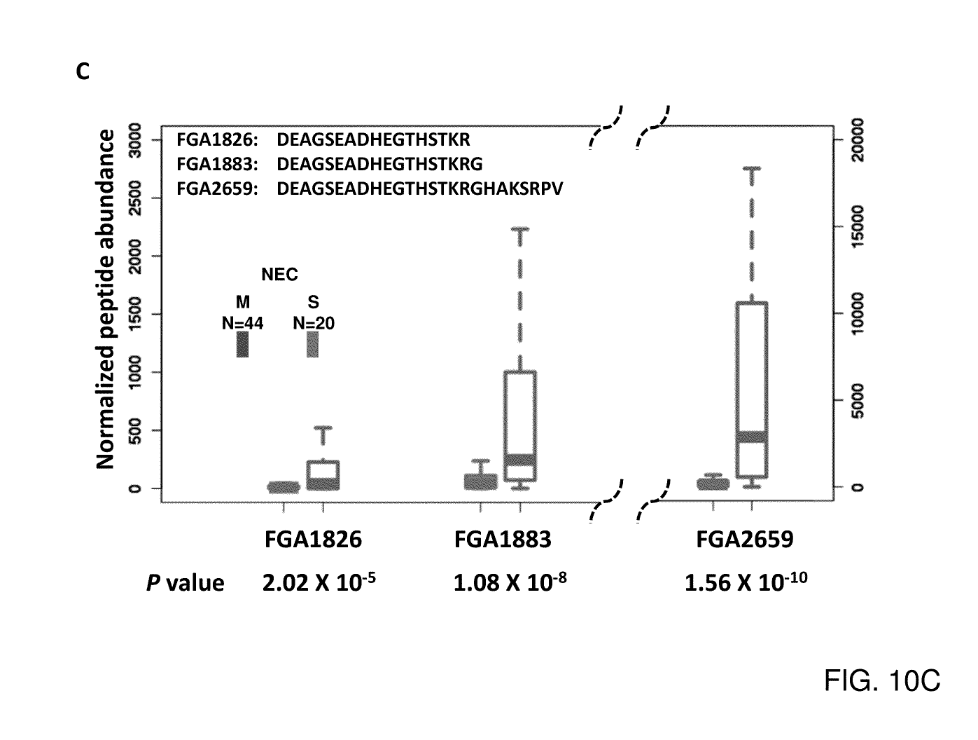

Sequence analysis of these NEC differentiating urine peptides, and their relative abundance represented by NSC values, revealed that several came from the same precursor proteins, and included (FIG. 3C) collagens (COL1A1, COL1A2, COL2A1), epithelial-mesenchymal cell interaction (EMI) domain-containing protein 1 (EMID1), Eps 15-Homology (EH) domain-binding protein 1-like protein 1 (EHBP1L1), fibrinogen alpha chain (FGA), gliding motility protein gliomedin (GLDN), hemoglobin subunit alpha (HBA1), Teneurin-3, PRAGMIN, steroidogenic factor 1 (SF1), and uromodulin (UMOD). For example, three different FGA peptides—DEAGSEADHEGTHSTKR, DEAGSEADHEGTHSTKRG, and DEAGSEADHEGTHSTKRGHAKSRPV—were detected. As discussed in greater detail in example 2 below, all three of these peptides could be validated by qualitative mass spectrometry (FIG. 10, and example 2).

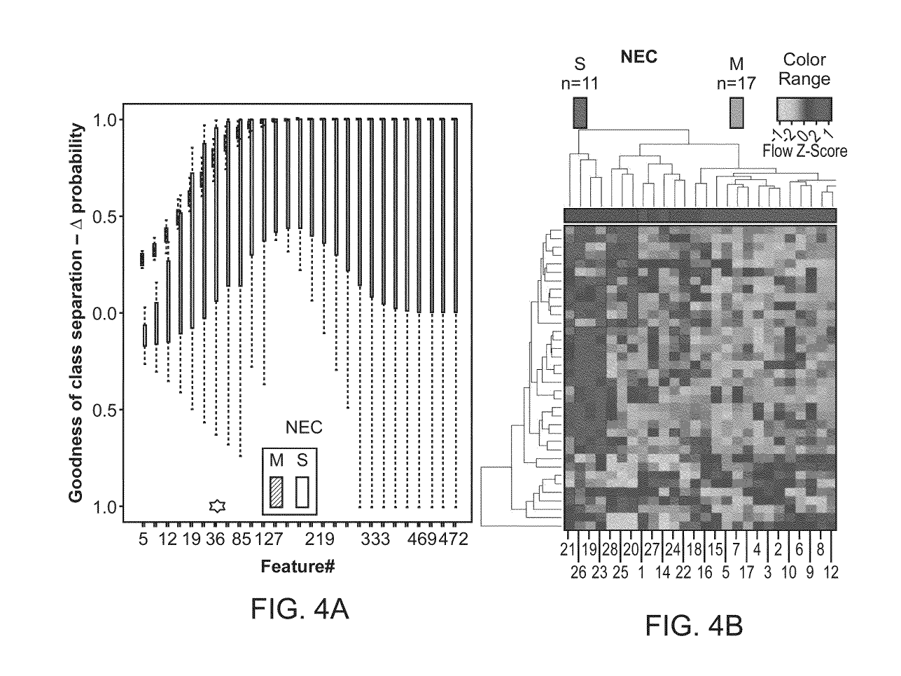

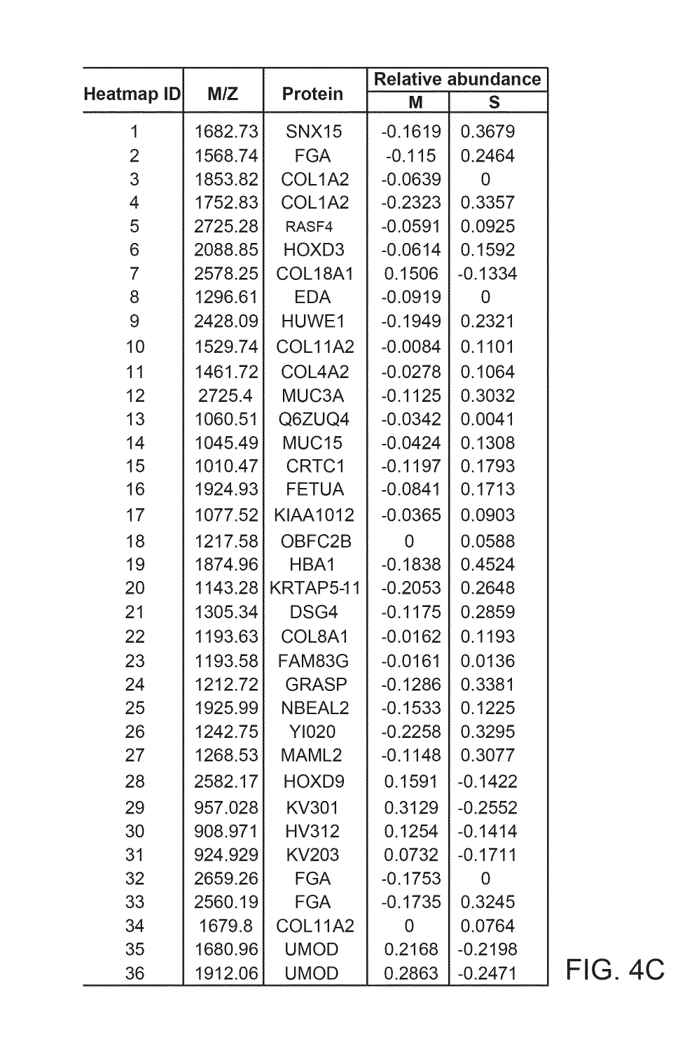

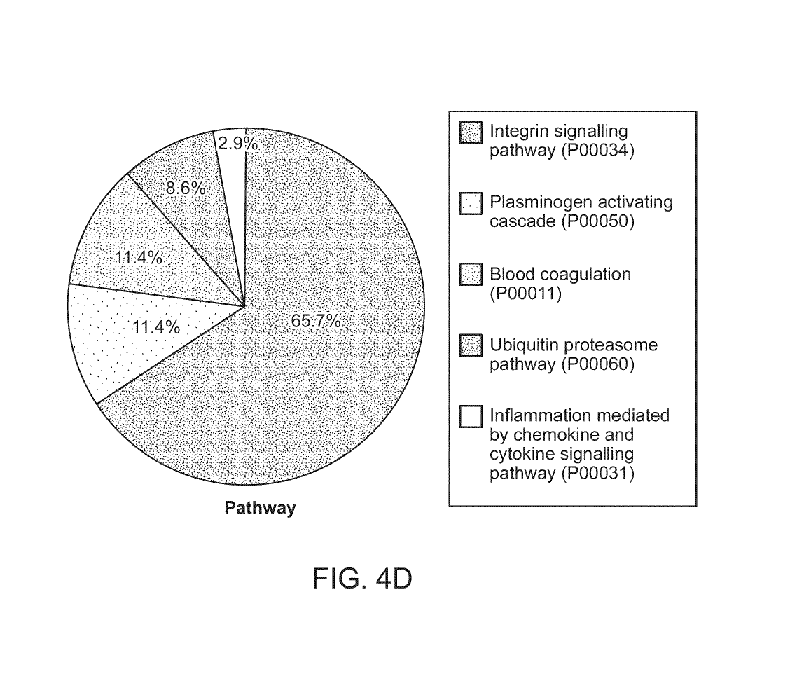

To develop a biomarker panel with manageable panel size, we built LDA classifiers with various subsets of the top ranked (NSC algorithm), therefore most significant, 473-peptide (sequence identified through MSMS analysis) data set. From these differentially expressed urine peptides, we sought to identify a biomarker panel of optimal feature number, balancing the need for small panel size, accuracy of classification, goodness of class separation (NEC M vs S), and with sufficient sensitivity and specificity. By goodness of separation, is meant the computed difference (Δ) between discriminative scores, calculated as estimated probabilities (Ling X B, et al. (2010) Integrative urinary peptidomics in renal transplantation identifies novel biomarkers for acute rejection. Journal of the American Society of Nephrology; Tusher V G, et al. (2001) Significance analysis of microarrays applied to the ionizing radiation response. Proc Natl Acad Sci USA 98(9):5116-5121). When class is predicted correctly, A probability is the difference of the highest and next highest probability; when predicted incorrectly, A probability is the difference of the probability of the true class and the highest probability, which will be negative. The NEC M and S box-whisker graphs are presented in FIG. 4A. Boxes contain 50% of values falling between the 25th and 75th percentiles; the horizontal line within the box represents the median value and the “whisker” lines extend to the highest and lowest values. This analysis revealed 36 peptides (FIG. 4C) to be the smallest panel size for which the “box” values of goodness of separation were positive for both NEC M and S.

To assess the association of the disease status with the abundance pattern of these 36 peptides, we performed unsupervised hierarchical cluster analysis with heat map plotting (FIG. 4B). The analysis demonstrated 2 major clusters reflecting NEC disease progression status, reinforcing the effectiveness of this 36-urine-peptide “signature” in predicting NEC M and S class distinction. Student T test and Mann-Whitney U test, in addition to MSMS sequence identification analyses (FIG. 4C) were performed for these 36 urine peptides. Close examination of these 36 peptides revealed nested peptides for COL1A2 (m/z 1853, 1752), COL11A2 (m/z 1529, 1679), FGA (m/z 1568, 2560, 2659), and UMOD (m/z 1680, 1912) having overlapping sequences derived from the same parent protein precursors. Further pathway analysis (FIG. 4D) using the PANTHER database (Mi H, et al. (2005) The PANTHER database of protein families, subfamilies, functions and pathways. Nucleic Acids Res 33(Database issue):D284-288 revealed these 36 peptide biomarkers derived from protein precursors involved in integrin signaling pathway (65.7%, P00034), plasminogen activating cascade (11.4%, P00050), blood coagulation (11.4%, P00011), ubiquitin proteosome pathway (8.6%, P0060), and inflammation mediated by chemokine and cytokine signaling pathway (2.9%, P00031) respectively. These findings are consistent with the presumed pathophysiology of exuberant inflammatory reaction resulting in coagulative necrosis of the gut wall.

Plasma Protein Panel Yields Effective Class Prediction for NEC M and S Patients.

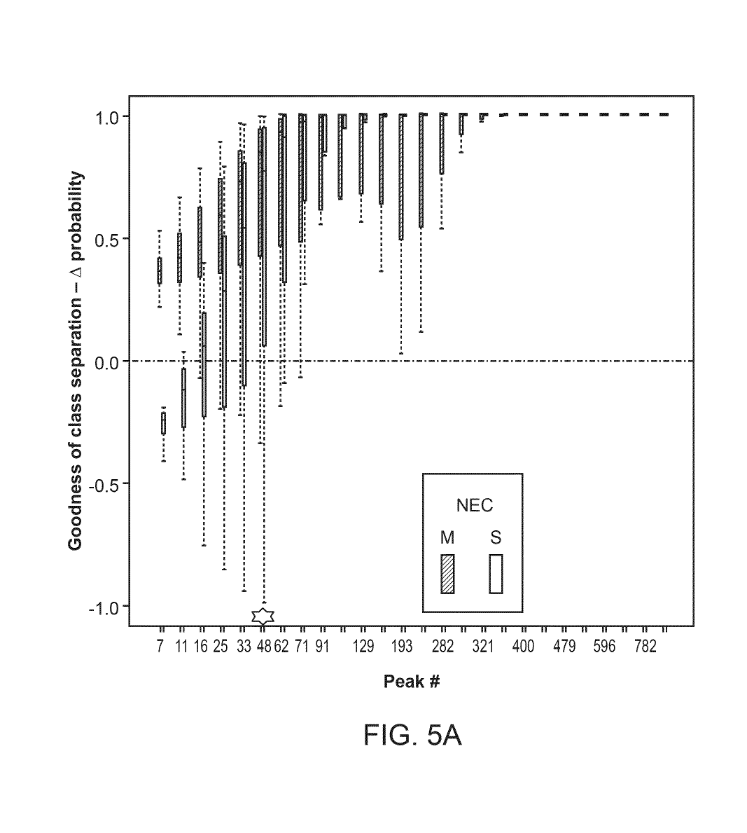

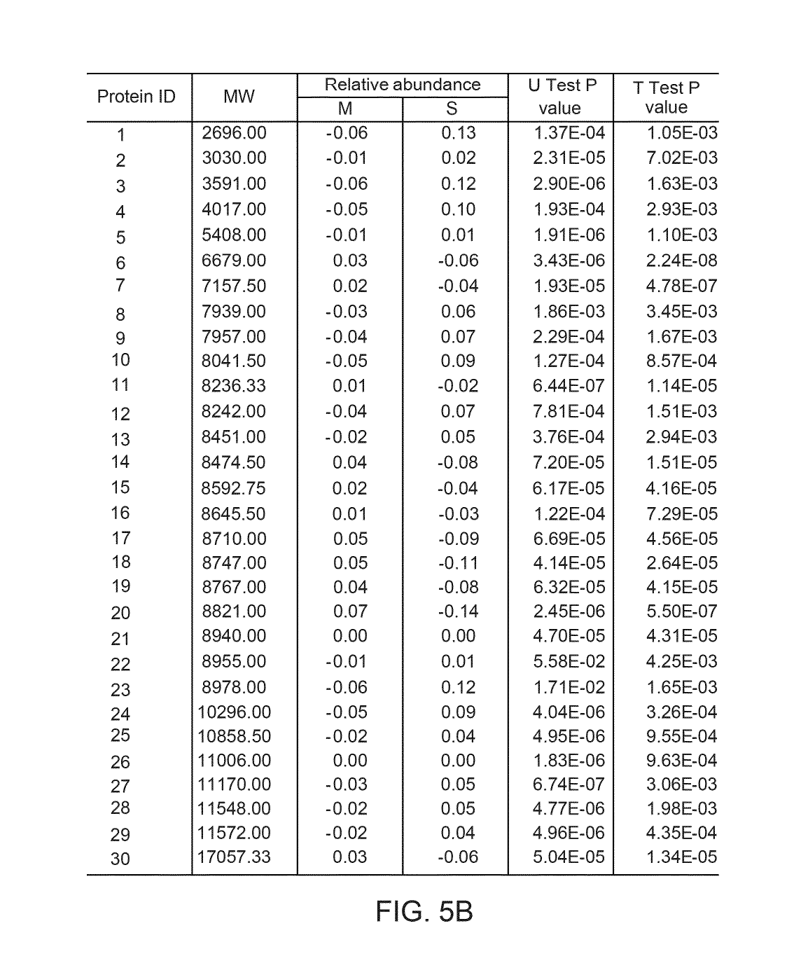

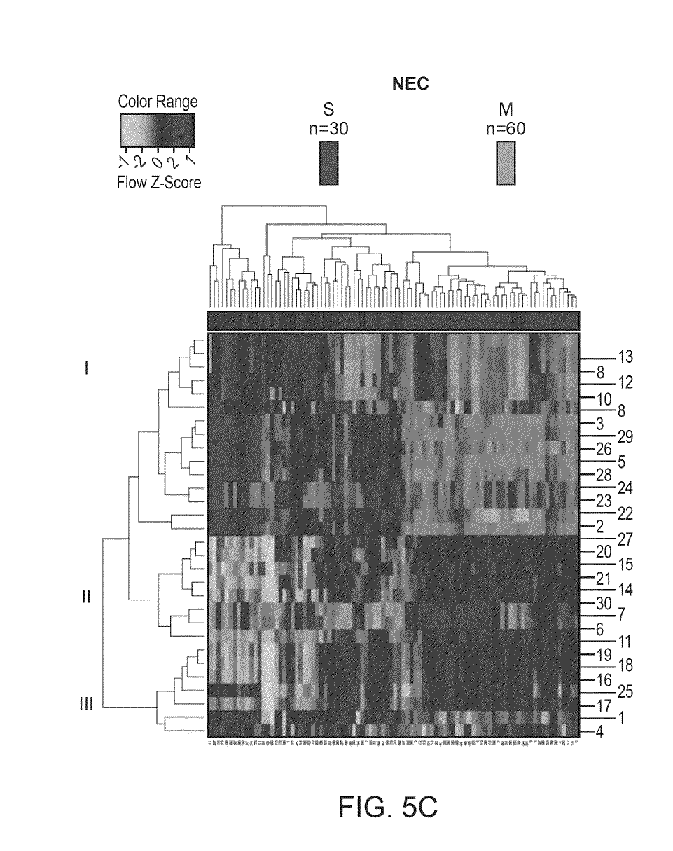

Patient blood samples were subject to SELDI-TOF MS based plasma proteomic analysis Carlson S M, Najmi A, Whitin J C, & Cohen H J (2005) Improving feature detection and analysis of surface-enhanced laser desorption/ionization-time of flight mass spectra. (Translated from eng) Proteomics 5(11):2778-2788 (in eng) (that resolved a total of 1528 protein peaks. All protein peaks were ranked by a nearest shrunken centroid (NSC) algorithm differentiating NEC M (n=60) and S (n=30) groups. As above, we sought to identify a biomarker panel of optimal features to achieve goodness of class separation (NEC M vs S), and with sufficient sensitivity and specificity. We built LDA classifiers with various subsets of the 1528-protein-peak data set. The computed goodness of separation (FIG. 5A) (defined above) is shown in FIG. 5A as the NEC M and S box-whisker graphs. As before, the boxes contain the interquartile range of values, the horizontal line within the box represents the median value and the “whiskers” extend to the highest and lowest values. This analysis revealed 48 to be the smallest panel size for which the “box” values of goodness of separation are positive for both NEC M and S. A close examination of the spectra revealed that these 48 spectral peaks actually are from 30 unique proteins (FIG. 5B). Relative abundance of the 30 plasma proteins (FIG. 5B) were analyzed by the nearest shrunken centroid values in either NEC M or S patient class with Color Scale conditional formatting. The significance of each plasma protein biomarker was quantified by Mann-Whitney U test and Student T test P values, demonstrating (reflecting) each plasma protein's individual effectiveness as a biomarker in differentiating NEC M from S groups. To assess the association of the disease status with abundance patterns of these 30 plasma proteins, we performed an unsupervised hierarchical cluster analysis with heat map plotting (FIG. 5C). The analysis shows NEC subjects clustered largely according to the disease progression status, reinforcing the effectiveness of this plasma-protein-peak “signature” in differentiating NEC M and S.

Comparative Analyses of Clinical and Molecular (Urine/Plasma) Based Biomarker Panels Via Unsupervised Learning.

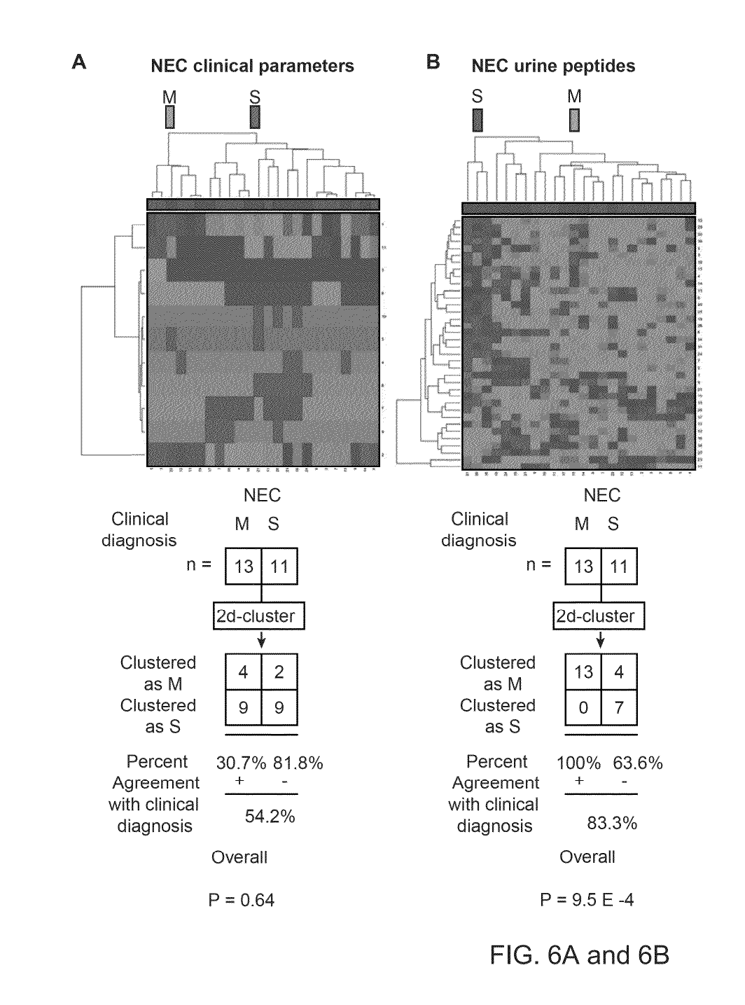

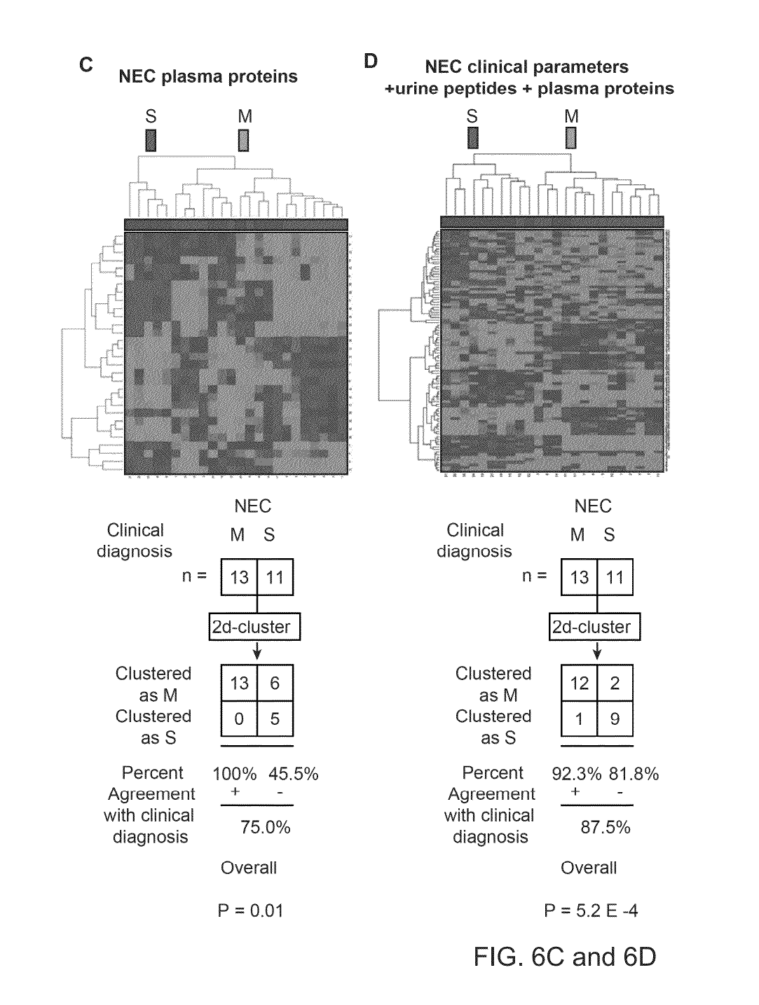

To compare the discriminant performance of different biomarker panels comprised of either 11 clinical parameters, 36 urine peptides, or 30 plasma proteins, a set of NEC patients (13 M and 11 S) were selected for which complete datasets of clinical findings, and urine/plasma profiling were available. Unsupervised cluster analyses were applied to determine how the NEC subjects were organized according to these clinical or molecular based biomarker classifiers. As shown in FIG. 6, each biomarker panel's differentiating pattern was represented by a corresponding cluster heat map. Recognizing the branch with the largest number of the clustered NEC S subjects as the NEC S “class” and the remaining as the NEC M “class”, the unsupervised discriminating significance of these different biomarker panels was quantified by the Fisher exact test of the 2×2 tables partitioning the clinically known subjects by the cluster grouping: clinical parameter panel (11 features), P value 0.64; urine peptide panel (36 features), P value 9.5×10⁻⁴; and plasma protein based panel (30 features), P value 0.01. The 36-urine-peptide panel appeared to be more effective than the 11-clinical-parameter or the 30-plasma-protein panel in discriminating NEC M from S subjects.

Integrative Analyses of Clinical and Molecular (Urine/Plasma) Findings Reveals an Optimal Biomarker Panel of 15 Urine Peptides and 3 Plasma Proteins for NEC Progression.

Through the unsupervised analysis, we were exploring whether an analysis integrating the clinical, urine peptide and plasma protein based biomarkers can achieve better predictive accuracy in NEC progression analysis. As shown in FIG. 6D, overall, with a P value of 5.2×10⁻⁴, the combined panel of 11 clinical parameters, 36 urine peptides and 30 plasma proteins correctly clustered 92.3% of NEC M and 81.8% NEC S subjects respectively, indicating greater effectiveness in NEC progression prediction for the integrated approach over any of the individual classifiers.

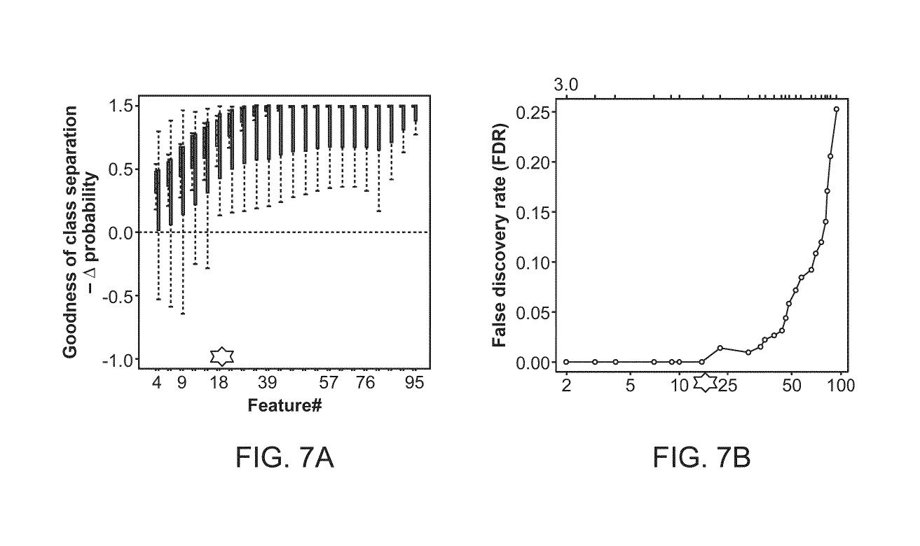

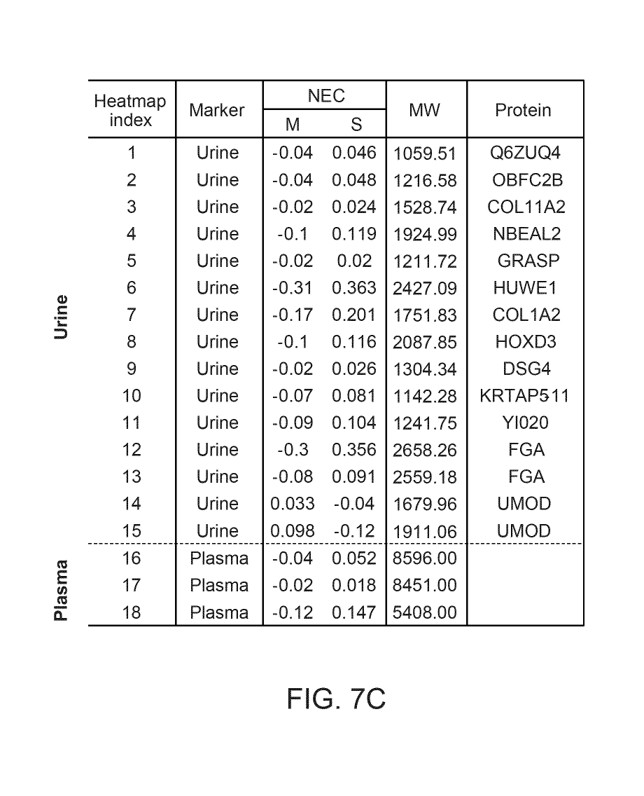

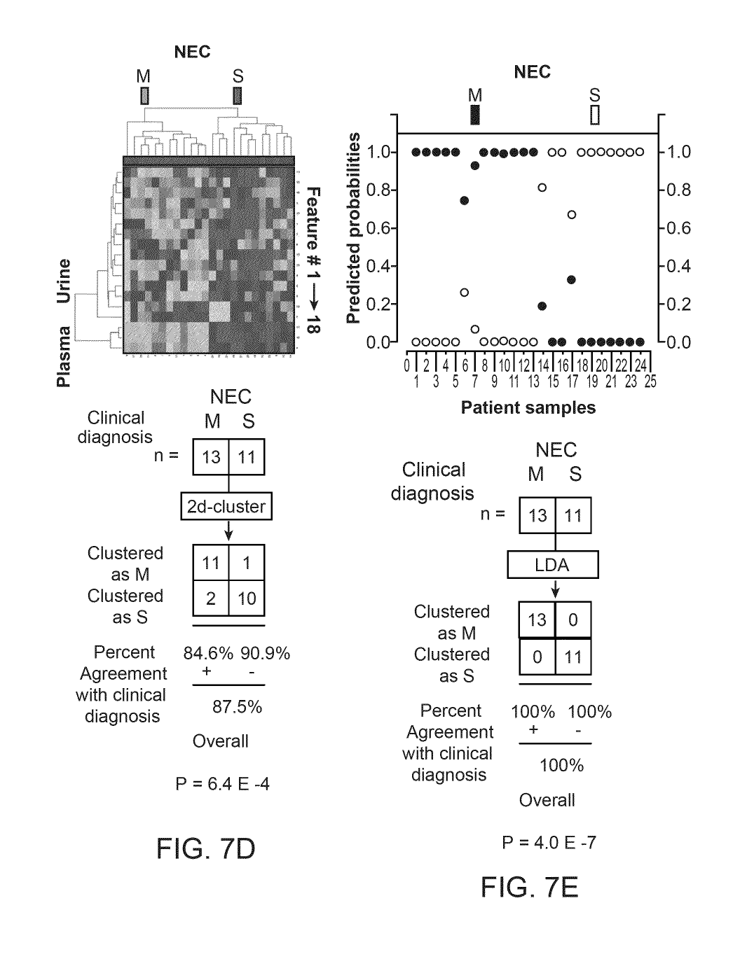

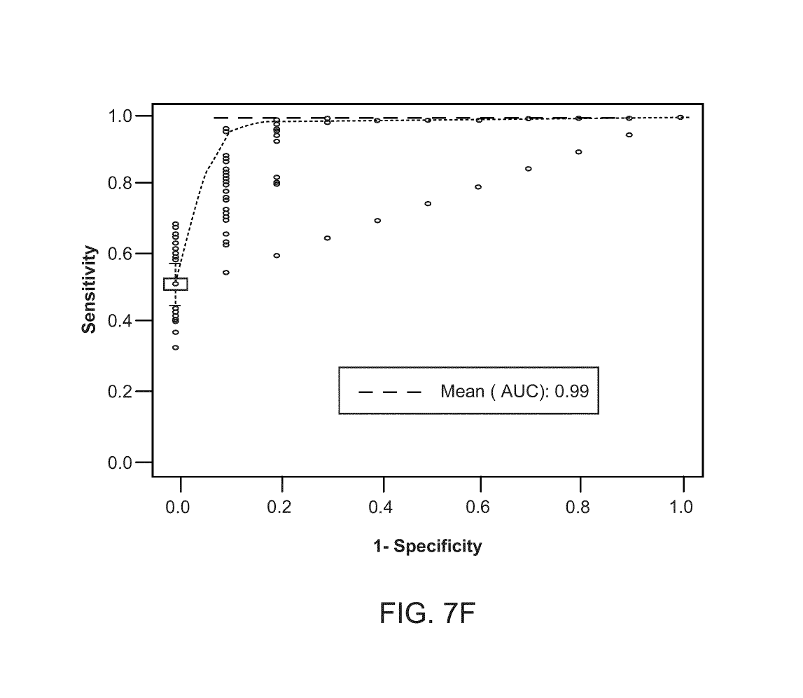

To find a predictive biomarker panel of optimal and manageable feature number, various subsets out of the combined biomarkers from different sources were tested as classifiers to analyze both their goodness of separation and false discovery rate (FDR). Linear discriminant probabilities of a biomarker panel of 18 features were found to be optimal for goodness of separation of the NEC M and S subjects (FIG. 7A). The FDRs of the LDA classifiers were estimated and were shown to significantly increase after the feature size expanded to greater than 18 (FIG. 7B). Therefore, the 18-feature biomarker panel was chosen as the optimal biomarker set, balancing the need for small panel size, accuracy of classification, goodness of class separation (NEC M versus S), and sufficient sensitivity and specificity. This 18-biomarker panel consisted of 15 urine peptides (corresponding to 13 proteins Q6ZUQ4, OBFC2B, COL11A2, NBEAL2, GRASP, HUWE1, COL1A2, HOXD3, DSG4, KRTAP5-11, Y1020, FGA, UMOD; close examination of the 15 urine peptides revealed the overlapping peptide fragments of FGA (MW: 2559, 2659) and UMOD (MW: 1679, 1911); see Table 11;) and 3 plasma peptides (CTAPIII, SAA1, B2M, TTR) The relative abundance of the 18 peptide biomarker panel (FIG. 7C) was analyzed by the nearest shrunken centroid values in either NEC M or S patient class and plotted with Color Scale conditional formatting.

An unsupervised analysis by heat map plotting across the 18 biomarkers demonstrated that all 11 of 13 NEC M subjects and importantly 10 of 11 NEC S subjects clustered together, co-clustered (FIG. 7D). The overall clustering agreement with clinical diagnosis is 87.5% and discriminant significance (P value) is 6.4×10⁻⁴. Using the 18-biomarker data set, supervised analysis was performed to develop the LDA model and the estimated probabilities were plotted (FIG. 7E). Samples were partitioned by the true class (upper) and predicted class (lower). The 2×2 contingency tables (FIG. 7E) summarizes the NEC M/S classification results, which are 100% agreeable with clinical diagnosis and P value of 4.0×10−7 by Fisher exact test. However, in order to avoid the problem of overfitting and bias, a bootstrapping method was used to resample the original 18-biomarker data set (NEC 13 M and 11 S subjects) 500 times, thus creating 500 new sets for LDA modeling and subsequent testing. For each of the bootstrapping sets, we used the LDA derived prediction scores of each sample to construct ROC curves. To summarize the 500 ROC analyses (FIG. 7F), box and whisker plots were used to describe the vertical spread around the median, and then the vertical average of the 500 ROC curves was plotted (dashed line). The ROC analyses yielded an average AUC of 0.99, demonstrating the robustness of the 18-biomarker panel in the discrimination of the NEC M and S class subjects.

A Sequential Ensemble Analysis of the Clinical and Molecular Biomarker Classifiers for Practical and Effective Prediction of NEC Progression.

Ensemble Data Mining Methods, also known as Committee Methods or Model Combiners, were used to combine the clinical and molecular biomarker classifiers in order to derive practical algorithms for NEC management. These machine learning methods leverage the power of multiple models to achieve better prediction accuracy than is possible with any of the individual models on their own. We integrated the molecular classifiers, either the 36 urine based or the final 18 (15 urine peptides and 3 plasma proteins) biomarker panel, with readily available clinical data. Using the 24 NEC subjects (13 M and 11 S) of which complete datasets are available, a simulation scenario—“NEC simulation set” was undertaken.

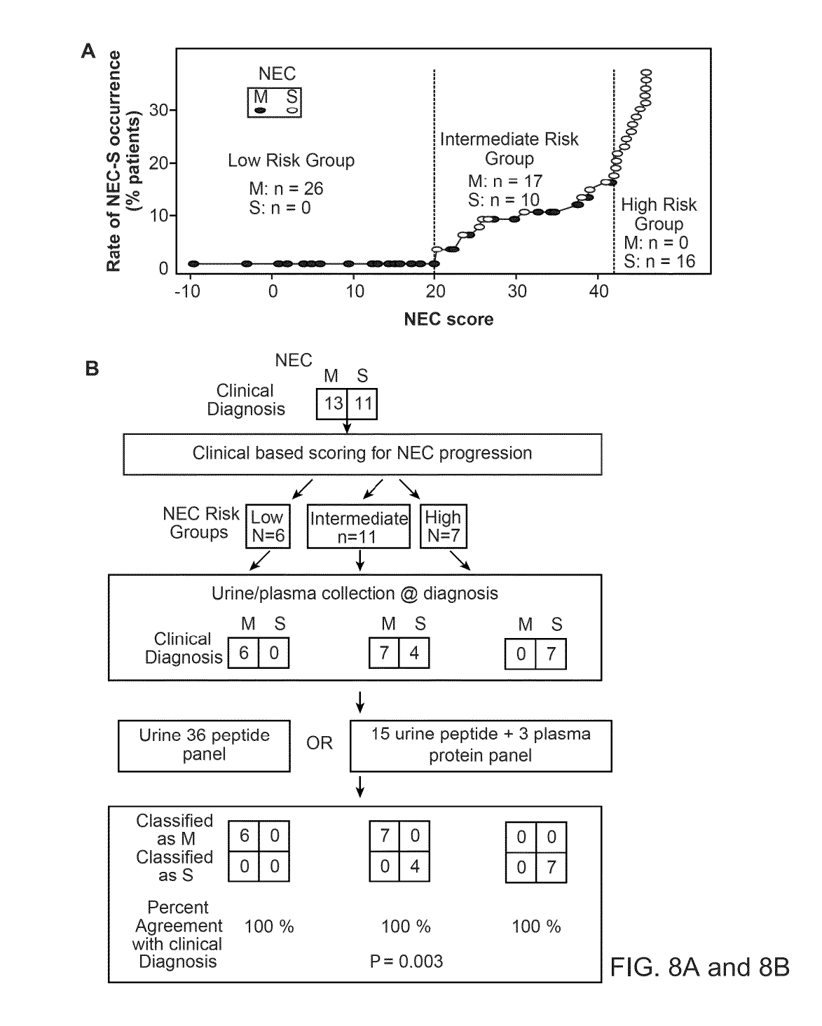

Based upon the multivariate analysis of the 11 clinical parameters of NEC 43 M and 26 S subjects (FIG. 2), NEC clinical scores were calculated ranging from −10 to 50 with a higher score indicating a greater chance or risk of NEC S. As shown in FIG. 8A, each particular sample's risk of being classified as NEC S was quantified by the proportion of NEC S samples with score less than that sample's clinical score in all NEC S samples. Therefore, all NEC samples were divided into low, intermediate, and high-risk groups based on their scores. A NEC clinical score of less than 20 classified samples into the low risk group, which produced a perfect match for the sub-group diagnosed as NEC M subjects (26 infants). A score of 42 or greater identified the high-risk group, in which all 16 infants were diagnosed as NEC S subjects. The remaining samples were grouped into the intermediate risk group, in which 17 were NEC M and 10 were NEC S subjects. Within the intermediate risk group, there are no clear delineations between NEC M and S subjects based simply on score. Therefore, we conclude that using the NEC clinical score, it is possible to stratify the NEC subjects into low (0%), intermediate (37%), and high-risk (100%) groups. If validated to be consistently demonstrable for NEC risk stratifications, the clinical score based forecast of the NEC subjects, particularly those into the low and high-risk bins, may be clinically useful to treat according to these prognostic indications.

Close examination of these subjects with comprehensive clinical, urine peptidomics and plasma proteomics data sets (FIG. 8B) found 6 in low, 11 in intermediate, and 7 in high-risk groups. These low or high risk subjects were ultimately diagnosed either as NEC M or S, reinforcing the notion there is a parallel relationship between the clinical diagnosis and the patient stratification by the NEC clinical scoring system. When tested further with either the 36 urine peptide panel or the final 18 biomarker panel, the classifications of these subjects in either low or high risk groups were in complete agreement with the clinical diagnosis, suggesting further molecular testing may be unnecessary due to the effective patient stratification by the NEC clinical based scoring system. However, as for the subjects in the intermediate groups assigned upon the NEC clinical scores, additional tests are needed to accurately classify the subjects and to predict NEC progression. For the NEC 7 M and 4 S subjects in the intermediate risk group, either the 36-peptide urine panel or the final 18-biomarker panel, classified them correctly with 100% agreement with clinical diagnosis and with a P value of 0.003 by the Fisher exact test. The simulation data set analysis suggests that the sequential and ensemble integration of the clinical and molecular based panels can adequately stratify patients to allow effective NEC management: (1) the low and high risk patients are correctly stratified for NEC progression by the clinical score; (2) the clinically intermediate risk patients are be subject to additional molecular based testing to produce further stratification thus allowing for the sensitive and specific prediction of NEC progression.

### Discussion

Necrotizing Enterocolitis (NEC) is a devastating inflammatory disease that affects at risk premature newborns in an un-predictable manner. NEC is a principal source of overall premature neonate mortality as well as short and long-term morbidity in surviving infants (7, 28). In general, NEC occurs in two forms that can be loosely described as non-progressive and progressive. These descriptive terms reflect the underlying degree of tissue injury that includes irreversible intestinal necrosis requiring its surgical removal. Despite numerous previous efforts, clinical parameters and serologic tests alone (Evennett N, et al. (2009) A systematic review of serologic tests in the diagnosis of necrotizing enterocolitis. J Pediatr Surg 44(11):2192-2201; Young C, Sharma R, Handfield M, Mai V, & Neu J (2009) Biomarkers for infants at risk for necrotizing enterocolitis: clues to prevention? Pediatr Res 65(5 Pt 2):91R-97R) appear to be inadequate for either diagnosing or predicting the outcome of NEC until late in the course of disease. Moreover, clinical signs of NEC, e.g. the x-ray finding of air or gas in the gut wall (pneumatosis intestinalis), are both non-specific of disease progression and vulnerable to observer variability and subjective assessment. Thus, the current approach to decision-making in treating NEC is generalized, non-specific and highly observer dependent. This is problematic, since 50% of cases will remain limited, and resolve with supportive care, while an additional 30-50% progress and require surgery. This leads to a number of both under and over-treated infants with likely effects on overall outcome (Cotten C M, et al. (2009) Prolonged duration of initial empirical antibiotic treatment is associated with increased rates of necrotizing enterocolitis and death for extremely low birth weight infants. Pediatrics 123(1):58-66). Novel therapeutic strategies that may ameliorate or halt progression of the disease cannot currently be tested since the only reliable signs of progressive NEC occur late in the course of disease when tissue destruction is irreversible and as such meaningful changes in patient care would therefore be unlikely of increased benefit. Moreover, since not all institutions caring for infants with NEC or at risk for NEC can offer surgery as a treatment (only highly specialized centers with neonatal and pediatric surgical sub-specialists), if those infants that are most likely to progress could be identified earlier, an option for transfer to a higher level of care center would be highly advantageous, and conversely, many transfers of infants not requiring surgery could be averted.

In this study we sought to address these challenges and have combined the novel use of available clinical data to effect an initial risk stratification of infants with NEC along with protein biomarker discovery. We report that the subsequent combination of these disparate datasets provides a useful and meaningful algorithm that correctly predicts NEC progression prior to the time at which obvious clinical signs of advanced disease are present. We conclude that this type of integrated and ensemble algorithm may overcome similar challenges encountered in other rare diseases that evolve either spontaneously or in response to therapy.

Like many other human diseases, NEC affects an organ system that is not readily amenable to biopsy to arrive at a definitive tissue diagnosis or prognosis. Thus, similar to other diseases, surrogate markers of disease (e.g. x-ray findings of pneumatosis intestinalis) or systemic signs (acidosis, white blood cell count) are currently utilized to risk stratify patients clinically. Various mass spectrometry based proteomics platforms are being increasingly applied to analyze available specimens (blood, urine, stool) in order to identify molecular markers of disease (biomarkers). In the current study, a robust set of several urine peptide biomarkers and plasma protein biomarkers enabled the accurate discrimination between NEC M and S urine samples. Several of these peptides were found to be derived from the same parent protein, indicating that either full-length polypeptide or peptide fragments thereof may be detected in the diagnostic and prognostic methods described herein. The finding of nested peptides is both reassuring and potentially informative since it would be unusual to discover various cleavage forms from the same parent protein as a spurious finding. Moreover, the nested peptides also suggest some novel aspects of the underlying biology of NEC. For example, since several of the identified peptides are derived from various collagens, collagen 1A2 (COL1A2), collagen 11A2 (COL11A2), this may reflect the possible involvement of specific exo- and endo-peptidases acting on the extra-cellular matrix (ECM) and potentially contributing to the underlying pathophysiology. Also interesting is the finding of COL4A2 (basement membranes), and MUC15 and MUC3A (cell surface glycoproteins expressed in enterocytes) all with increased relative expression in the NEC S class of patients. Together, these peptides more specifically point toward a destructive process in the gut with perhaps cell surface or basement membrane breaching of the intestinal epithelium which has been proposed by several authors as contributing significantly to the pathogenesis of NEC (Hackam D J, et al. (2005) Disordered enterocyte signaling and intestinal barrier dysfunction in the pathogenesis of necrotizing enterocolitis. Semin Pediatr Surg 14(1):49-57; Anand R J, et al. (2007) The role of the intestinal barrier in the pathogenesis of necrotizing enterocolitis. Shock 27(2):124-133).

One persistent finding that consistently survived all of the analyses was that of increased FGA (fibrinogen, alpha chain) peptides in the NEC S class patient urine. FGA is involved in tissue injury and blood coagulation as the most abundant component of thrombus formation. Liquefaction necrosis with significant small vessel thrombosis is a common pathologic finding in surgical NEC. In addition, various cleavage products of fibrinogen can regulate cell adhesion, display vasoconstrictor and chemotactic activities, and are mitogens for several cell types. Other significant collagens of potential biologic significance include collagen 8A1 (COL8A1) a component of vascular endothelium, and collagen 18A1 (COL18A1), also involved in the coagulation cascade. The consistent finding of peptides derived from uromodulin, the most abundant protein in normal urine, suggests a systemic inflammatory injury since uromodulin is not derived from the plasma, but rather is produced in the glomeruli. The proteolytic cleavage of an ectodomain of uromodulin on the luminal surface in the loop of Henle and its urinary secretion suggests secondary systemic effects as a result of the remote gut disease. Together, these various peptides suggest that peptide biomarkers may serve as surrogates of disease-related protease/protease inhibitors (e.g. TIMP1, MMPs) that may be differentially active in the two classes of NEC thereby reflecting the underlying tissue destruction. For example, the identification of urine peptide biomarkers suggests that active degradation of collagen is associated with the pathophysiology of NEC progression. This is in line with our previous findings that nested urinary peptide biomarkers may be generated by disease-specific exo-peptidase activity (Villanueva J, et al. (2006) Differential exoprotease activities confer tumor-specific serum peptidome patterns. J Clin Invest 116(1):271-284).

The present sequential ensemble analyses leverages the power of the findings of both the molecular (urine peptidome and plasma proteome) and the clinical parameters based biomarker panels to achieve better accuracy in predicting the progression of NEC to an advanced stage of disease. The derivation of the scoring metrics for NEC clinical parameter-based predictions further enable the biomarker panel to be interpreted on a scale, which increases the flexibility of the panel to quantify the risk of NEC progression. The complementary effectiveness of our integrative diagnostic analysis may reflect the complex pathophysiology of NEC with diverse and interdependent clinical and biological variables. Our analyses and algorithm also suggest a potential strategy to be utilized for numerous other diseases. Diseases that can be stratified by clinical parameters and then further sub-stratified by validated biomarkers may particularly benefit. Taken together this approach and the findings presented demonstrate the additive power of integrating data from various sources

### Example 2

### Methods

Patient Population.

This study was approved by the human subjects' protection programme at each participating institution (Stanford protocol ID 23091). Informed consent was obtained from the parents of all enrolled subjects. Patient contributions by institution included: Baylor/Texas Children's Hospital (n=184), Yale-New Haven Children's Hospital (n=158), UCSF/Benioff Children's Hospital (n=79), Boston Children's Hospital (n=75), UCLA/Mattel Children's Hospital (n=42), Johns Hopkins Children's Center (n=22), Stanford/Lucile Packard Children's Hospital (n=16) and Children's Hospital of Philadelphia (n=11). Complete data collection including patient-specific demographic, clinical and laboratory data were prospectively collected from a total of 550 infants. Those with incomplete data collection were excluded from the study.

Urine samples were collected from a subset of 65 infants with suspected NEC. Patient contributions by institution included: Baylor/Texas Children's Hospital (n=16), Yale-New Haven Children's Hospital (n=24), Johns Hopkins Children's Center (n=17), Stanford/Lucile Packard Children's Hospital (n=4) and Children's Hospital of Philadelphia (n=4). The samples were collected at the time of initial clinical concern for NEC. The infants were then followed clinically and ultimately categorised as either medical NEC (improved without surgery) or surgical NEC (required laparotomy, peritoneal drainage or died from complications of NEC prior to intervention). The urine samples were then compared between the medical NEC and surgical NEC groups for all subsequent analyses.

For the development of the clinical parameter-based prognostic algorithm 485 infants were randomised into two cohorts for statistical training (n=323) and testing (n=162). For the urine peptidome analysis the remaining 65 infants were assigned to either the biomarker discovery cohort (n=28) or the biomarker validation cohort (n=37). Comparative demographic analyses were performed by Cochran Mantel-Haenszel x2 and analysis of variation (ANOVA) with adjustment for institution (R epicalc package).

### Clinical Parameter-Based Prognostic Algorithm

The clinical parameters for the infants randomised to the statistical training cohort (n=323) were analysed by linear discriminant analysis (LDA) using the R library MASS function ‘Ida’ (http://www.r-project.org/). All subjects in the training cohort were subsequently assigned to one of three possible subgroups (low-risk, indeterminate or high-risk) based on 95% correct classification in the low-risk (5% probability of actually being surgical NEC) and in the high-risk (5% probability of actually being medical NEC) groups. This process was then repeated on the testing cohort (n=162). The prognostic characteristics of the clinical parameters were then subjected to receiver-operator characteristic (ROC) analyses of their ability to differentiate infants with medical NEC from those with surgical NEC.

### Urine Biomarker-Based Prognostic Algorithm

Biomarker discovery and validation overview. 65 subjects were assigned to either the biomarker discovery cohort (n=28) or the biomarker validation cohort (n=37). Urine sample collection, processing and peptide extraction were performed according to previously described protocols (Ling X B, et al. A diagnostic algorithm combining clinical and molecular data distinguishes Kawasaki disease from other febrile illnesses. BMC Med 2010; 9:130-41; Ling X B, et al. Urine peptidomics for clinical biomarker discovery. Adv Clin Chem 2010; 51:181-213; Ling X B, et al. Integrative urinary peptidomics in renal transplantation identifies biomarkers for acute rejection. J Am Soc Nephrol 2010; 21:646-53). Liquid chromatography—matrix-assisted laser desorption/ionisation mass spectrometry (MS) (LC-MALDI, ABI 4700, Applied Biosystems, California, USA) was used for comprehensive analysis of the urine peptidomes. Biomarker validation was performed by repeat, confirmatory analysis of the initial 28 infants in the discovery cohort followed by analysis of the 37 patients in the naïve validation cohort using multiple reaction monitoring (MRM) assays conducted on a triple quadrupole mass spectrometer (Quattro Premier, Waters Corporation, Massachusetts, USA).

Details of Biomarker Discovery.

A comprehensive urine peptidome analysis was performed using a label-free approach. This entailed first selecting biomarker candidate MS peaks on the basis of discriminant analysis and then targeting candidate biomarkers for tandem MS (MS/MS) sequencing analysis to identify the peptide sequences of interest. The in-house informatics platform, ‘MASS-Conductor’, was employed. This platform consists of an integrated suite of algorithms and statistical methods to allow comprehensive analysis of LC-MALDI-based urine peptide profiling as previously described (Tibshirani R, et al. Sample classification from protein mass spectrometry, by ‘peak probability contrasts’. Bioinformatics 2004; 20:3034-44; Yasui Y, et al. A data-analytic strategy for protein biomarker discovery: profiling of high-dimensional proteomic data for cancer detection. Biostatistics 2003; 4:449-63; Tibshirani R, et al. Diagnosis of multiple cancer types by shrunken centroids of gene expression. Proc Natl Acad Sci USA 2002; 99:6567-72).

To confirm the identity of the candidate peptide biomarkers, extensive MALDI-time of flight (TOF)/TOF and linear trap quadrapole Orbitrap MS/MS analyses were employed, coupled with database searches as previously described (Ling X B, et al. A diagnostic algorithm combining clinical and molecular data distinguishes Kawasaki disease from other febrile illnesses. BMC Med 2010; 9:130-41; Ling X B, et al. Urine peptidomics for clinical biomarker discovery. Adv Clin Chem 2010; 51:181-213; Ling X B, et al. Integrative urinary peptidomics in renal transplantation identifies biomarkers for acute rejection. J Am Soc Nephrol 2010; 21:646-53; Ling X B, et al. Urine peptidomic and targeted plasma protein analyses in the diagnosis and monitoring of systemic juvenile idiopathic arthritis. Clin Proteomics 2010; 6:175-93). All features were ranked by a nearest shrunken centroid algorithm to optimize the differentiation between the medical NEC and the surgical NEC groups (Tusher V G, et al. Significance analysis of microarrays applied to the ionizing radiation response. Proc Natl Acad Sci USA 2001; 98:5116-21)

Details of Biomarker Validation.

Following the discovery of candidate peptide biomarkers, MRM assays were performed as previously described (Ling et al. Integrative urinary peptidomics in renal transplantation identifies biomarkers for acute rejection. J Am Soc Nephrol 2010; 21:646-53). Stable isotope-labelled peptides (with a 13C-labelled amino acid) were synthesized and used as internal standard peptides. MRM measurement was normalized to each sample's total peptide content (TNBS assay) for further data analysis. The performance of the urine peptide classifiers using the MRM measurements were assessed and visualized by receiver-operating characteristic curve ROCR package (Sing T, et al. ROCR: visualizing classifier performance in R. Bioinformatics 2005; 21:3940-1).

Ensemble Algorithm Combining Clinical Parameters and Urine Biomarkers.

LDA was used to classify individual subjects based on clinical findings. Urine peptides were validated using the R library MASS function ‘Ida’. ROC analyses of the predictive performance was performed. The projection value onto the first canonical (LDA) was designated as the NEC outcome score, allowing the clinical parameters and fibrinogen (FGA) urine peptides to be collectively interpreted on a scale, rather than a strict binary discrimination.

### Results

Patient Characteristics.

Basic patient characteristics and demographics are shown in tables 1 and 2. Observed trends for gender, gestational age and birth weight exist with the surgical NEC cohorts tending to be men of younger gestational age and lower birth weight. These trends reached statistical significance only with regards to patient gender (in the clinical algorithm and biomarker groups, tables 8 and 9) and birth weight (in the clinical algorithm group, table 8), and are likely of little clinical significance as such trends are frequently observed in infants with NEC.

TABLE 8
Patient characteristics for necrotising enterocolitis (NEC) clinical
outcome algorithm development. Patients had the opportunity
to report as Hispanic in addition to the other race identifiers.

| | Training | | | Testing | | |
|---|---|---|---|---|---|---|
| | Medical | Surgical | | Medical | Surgical | |
| | NEC | NEC | | NEC | NEC | |
| | n = 230 | n = 93 | p | n = 115 | n = 47 | p |
| | (71.2%) | (28.8%) | Value | (71.0%) | (29.0%) | Value |
| Male* | 125 | 61 | 0.048 | 56 | 33 | 0.020 |
| | (54.4%) | (65.6%) | | (48.7%) | (70.2%) | |
| Gestational | 29.8 (29.3 | 28.6 (27.8 | 0.055 | 30.2 (29.4 | 29.5 (28.2 | 0.559 |
| age (weeks)† | to 30.3) | to 29.4) | | to 30.9) | to 30.7) | |
| Birth weight | 1376.3 | 1142.1 | 0.029 | 1418.8 | 1329.6 | 0.625 |
| (grams)† | (1283.0 to | (995.4 to | | (1275.4 to | (1104.9 to | |
| | 1469.5) | 1288.8) | | 1562.1) | 1554.3) | |
| Race* | | | 0.301 | | | 0.959 |
| Caucasian | 119 | 41 | | 52 | 22 | |
| | (51.7%) | (44.1%) | | (45.2%) | (46.8%) | |
| African | 65 | 31 | | 39 | 18 | |
| American | (28.3%) | (33.3%) | | (33.9%) | (38.3%) | |
| Hispanic | 55 | 26 | | 27 | 14 | |
| | (23.9%) | (28.0%) | | (23.5%) | (29.8%) | |
| Asian | 8 | 2 | | 4 | 1 | |
| | (3.5%) | (2.2%) | | (3.5%) | (2.1%) | |
| Native | 0 | 2 | | 1 | 0 | |
| Hawaiian | (0%) | (2.2%) | | (0.9%) | (0%) | |
| or Pacific | | | | | | |
| Islander | | | | | | |
| American | 2 | 0 | | 0 | 0 | |
| Indian or | (0.9%) | (0%) | | (%) | (0%) | |
| Alaskan | | | | | | |
| Native | | | | | | |
| Unknown | 30 | 13 | | 16 | 5 | |
| | (13.0%) | (14.0%) | | (13.9%) | (10.6%) | |
| Other | 6 | 4 | | 3 | 1 | |
| | (2.6%) | (4.3%) | | (2.6%) | (2.1%) | |

*Fischer's exact test; percentages in parentheses.
†ANOVA; least square mean is reported with 95% Cl in parentheses.

TABLE 9
Patient characteristics for necrotising enterocolitis (NEC)
biomarker algorithm development. Patients had the opportunity
to report as Hispanic in addition to the other race identifiers.

| | Discovery | | | Validation | | |
|---|---|---|---|---|---|---|
| | Medical | Surgical | | Medical | Surgical | |
| | NEC | NEC | | NEC | NEC | |
| | n = 17 | n = 11 | p | n = 27 | n = 10 | p |
| | (60.7%) | (39.3%) | Value | (73.0%) | (27.0%) | Value |
| Male* | 7 | 10 | 0.025 | 12 | 5 | 0.763 |
| | (41.2%) | (90.9%) | | (44.4%) | (50.0%) | |
| Gestational | 28.9 (27.3 | 28.0 (25.9 | 0.236 | 31.9 (24.0 | 29.4 (23.0 | 0.873 |
| age (weeks)† | to 30.6) | to 30.1) | | to 40.0) | to 38:0) | |
| Birth weight | 1230.5 | 1167.9 | 0.609 | 1834.9 | 1470.1 | 0.457 |
| (grams)† | (917.3 to | (778.6 to | | (598.0 to | (540.0 to | |
| | 1543.7) | 1557.3 | | 4150.0) | 2951.0) | |
| Race* | | | 0.145 | | | 0.598 |
| Caucasian | 12 | 4 | | 15 | 4 | |
| | (70.6%) | (36.3%) | | (55.5%) | (40.0%) | |
| African | 3 | 5 | | 9 | 4 | |
| American | (17.7%) | (45.5%) | | (33.3%) | (40.0%) | |
| Hispanic | 2 | 3 | | 0 | 0 | |
| | (11.8%) | (27.3%) | | (0%) | (0%) | |
| Asian | 2 | 0 | | 1 | 0 | |
| | (11.7%) | (0%) | | (3.7%) | (0%) | |
| Native | 0 | 0 | | 0 | 0 | |
| Hawaiian | (0%) | (0%) | | (0%) | (0%) | |
| or Pacific | | | | | | |
| Islander | | | | | | |
| American | 0 | 0 | | 0 | 0 | |
| Indian or | (0%) | (0%) | | (0%) | (0%) | |
| Alaskan | | | | | | |
| native | | | | | | |
| Unknown | 0 | 0 | | 0 | 0 | |
| | (0%) | (0%) | | (0%) | (0%) | |
| Other | 0 | 0 | | 2 | 2 | |
| | (0%) | (0%) | | (7.5%) | (20.0%) | |

*Fischer's exact test; percentages in parentheses.
†ANOVA; least square mean is reported with 95% Cl in parentheses.

The time between initial clinical concern (the time of urine sample collection) and confirmed medical NEC, defined as the presence of pneumatosis, was median 31 h (IQR 10, 63). The time between initial clinical concern and confirmation of surgical NEC, defined as the time of laparotomy, peritoneal drain or death from complication of NEC, was median 57 h (IQR 17, 213). There were no NEC-related deaths in the medical NEC cohort (n=389) and the combined mortality rate for the surgical NEC cohort was 27.9% (45/161).

### Effectiveness of Clinical Parameter-Based Prognostic Algorithm.

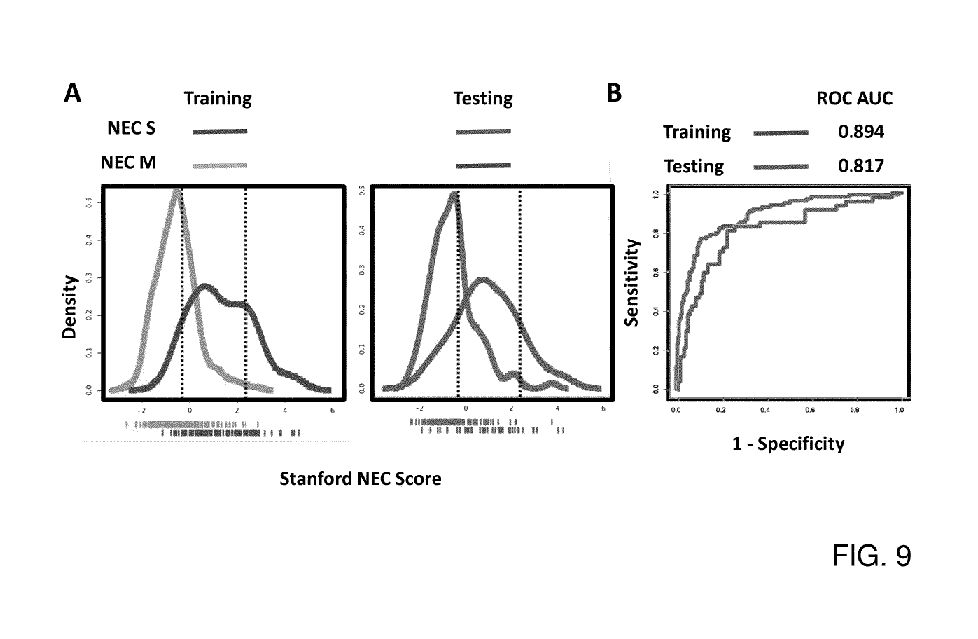

The LDA-based model risk stratified all subjects in training and testing cohorts into the three levels of risk for progression as discussed above (low-risk, indeterminate and high-risk). Twenty-seven clinical parameters were used in the LDA analysis based on the coefficients of linear discriminants as listed in table 10. The LDA clinical risk stratification algorithm could not confidently predict the outcome for 42.4% and 40.1% of training and testing subjects respectively—percentages representing the proportion of infants remaining in the indeterminate group (FIG. 9A). ROC analysis and calculated area under the curves (AUCs) for the outcome prediction of medical NEC or surgical NEC were 0.894 and 0.817 in the training and testing cohorts, respectively (FIG. 9B).

TABLE 10
Clinical parameters ordered by contribution (weight, LD1) to the necrotising
enterocolitis (NEC) outcome linear discriminant analysis (LDA) model.
ANC, absolute neutrophil count; BAND, band neutrophil; LD1, coefficient
of linear discriminant; WBC, white blood cell count.

| Diagnostic criteria | LD1 |
|---|---|
| pH value | −2.94E+3000 |
| Portai venous gas? | 1.66E+3000 |
| Air/fluid levels? | 7.71E−01 |
| Thrombocytopenia | 7.33E−01 |
| On a ventilator on the day protocol definition of | 6.94E−01 |
| NEC was met? | |
| Abdominal distention? | 5.91E−01 |
| Abdominal tenderness? | 4.82E−01 |
| Neutropenia | 4.60E−01 |
| Abdominal wall discoloration? | 4.17E−01 |
| Feeding intolerance? | 3.95E−01 |
| Pneumatosis intestinalis? | 3.87E−01 |
| Apneic/bradycardic episode? | −3.06E−01 |
| Acidosis | 2.69E−01 |
| Dilated bowel? | 2.59E−01 |
| pH site | −2.34E−01 |
| On vasopressors on the day protocol definition of | −1.32E−01 |
| NEC was met? | |
| Capillary refill time greater than 2 s? | 9.12E−02 |
| ileus present? | −6.67E−02 |
| Oxygen desaturation episode? | −6.48E−02 |
| ANC (neutrophil counts) | 5.53E−02 |
| Grossly bloody stools? | 5.50E−02 |
| WBC (×10³/mm³) | −2.83E−02 |
| Thickened bowel walls? | −1.80E−02 |
| BAND % | 1.47E−02 |
| Neutrophils (%) | 1.09E−02 |
| Bicarbonate (meg/L) | 6.55E−04 |
| Platelets (×103/μL) | 3.52E−04 |

### Effectiveness of Biomarker-Based Prognostic Algorithm

Biomarker Discovery.

As discussed above in example 1, the MALDI-TOF MS analysis of the urine samples from the infants in the biomarker discovery cohort resolved a total of 17,173 peptide peaks defined by distinct mass to charge ratio and high performance liquid chromatography fractions in the 900-4000-Da range. The nearest shrunken centroid algorithm then identified the most significant 473 peptides (sequence identified through MSMS analysis). A LDA model was then implemented to identify a biomarker panel of optimal feature number by balancing the need for small panel size, accuracy of classification, goodness of class separation (medical NEC vs surgical NEC), and with sufficient sensitivity and specificity. As discussed above, this analysis revealed an optimum 36-peptide panel (FIG. 4C) for which the probability scores indicated goodness of class separation for medical NEC and surgical NEC (FIG. 4A). Unsupervised hierarchical cluster analysis with heat map plotting was then used to visually depict the association of the disease status with the abundance pattern of these peptides (FIG. 4B). This analysis demonstrated two major clusters reflecting NEC disease progression status, reinforcing the effectiveness of this urine peptide ‘signature’ in predicting medical NEC and surgical NEC class distinction.

Student t test and Mann-Whitney U test, in addition to MSMS sequence identification analyses (table 11; see also FIG. 7C) were performed for these urine peptides.

TABLE 11
Necrotising enterocolitis (NEC) outcome predictive urine peptide
biomarkers revealed by LC-MALDI urine peptidome profiling.
Relative abundance

| | Medical | Surgical | MW | Protein | Sequence |
|---|---|---|---|---|---|
| 1 | −0.04 | 0.046 | 1060.51 | Q6ZUQ4 | S.CKSPAQ@RRGG.S |
| 2 | −0.04 | 0.048 | 1217.58 | OBFC2B | S.QP#NHTP#AGPP#GP.S |
| 3 | −0.02 | 0.024 | 1529.74 | COL11A2 | D.VGPMGP#PGPPGP#RGPAG.P |
| 4 | −0.1 | 0.119 | 1925.99 | NBEAL2 | Q.SVPASTGLGWGSGLVAPLQE.G |
| 5 | −0.02 | 0.02 | 1212.72 | GRASP | P.P#ALPPPPPP#ARA.F |
| 6 | −0.31 | 0.363 | 2428.09 | HUWE1 | P.GP*SPGTGPGP*GP*GP*GPGPGPGPGPGPGPGP.G |
| 7 | −0.17 | 0.201 | 1752.83 | COL1A2 | A.GEKGPSGEAGTAGPP*GTP*GP.Q |
| 8 | −0.1 | 0.116 | 2088.85 | HOXD3 | P.GN@HHHGP#CDPHP#TYTDLSA.H |
| 9 | −0.02 | 0.026 | 1305.34 | DSG4 | L.YACDCDDNHM#C.L |
| 10 | −0.07 | 0.081 | 1143.28 | KRTAP5- | P.CCSSSGCGSFCC.Q |
| | | | | 11 | |
| 11 | −0.09 | 0.104 | 1242.75 | YI020 | R.PKPSPPPPLILS.P |
| 12 | −0.3 | 0.356 | 2659.26 | FGA | A.DEAGSEADHEGTHSTKRGHAKSRPV.R |
| 13 | −0.08 | 0.091 | 2560.18 | FGA | A.DEAGSEADHEGTHSTKRGHAKSRP.V |
| 14 | 0.033 | −0.04 | 1680.96 | UMOD | S.VIDQSRVLNLGPITR.K |
| 15 | 0.098 | −0.12 | 1912.06 | UMOD | R.SGSVIDQSRVLNLGPITR.K |

Relative abundance: PAM algorithm derived shrunken difference, derived by shrinking the class centroids toward the overall centroids after standardizing by the within-class SD, for the 15 peptides between medical NEC and surgical NEC subjects.
MW, molecular weight.
Q@ = Deamidation of Glutamine;
P* = Hydroxylation of Proline;
P# = Oxidation of Proline;
N@ = Deamidation of Asparagine;
M# = Methionine sulfoxide.
“.” = the border of the peptide sequence. For example, for peptide 1, the detected sequence is CKSPAQ@RRGG, with a deamidated glutamine at position 6.

We then examined the list of candidate urine peptide biomarkers and the associated signaling pathways that define their biology. As discussed above, pathway analysis (FIG. 4D) using the PANTHER database revealed the candidate peptide biomarkers to be principally involved in integrin signalling (65.7%), plasminogen activating cascade (11.4%), blood coagulation (11.4%), ubiquitin proteasome pathway (8.6%) and inflammation mediated by chemokine and cytokine signalling pathway (2.9%). Moreover, sequence alignment of the candidate peptides revealed tight sequence clusters for two fibrinogen A (FGA2560, FGA2659) and two uromodulin (UMOD 1680, UMOD 1912) peptides. Given the biological plausibility and peptide homogeneity, these peptides were selected for further validation on the naïve biomarker validation cohort.

Biomarker Validation.

Prior to validation on the naïve cohort, MRM was used for quantitative confirmation of the FGA and UMOD cluster peptides in urine samples of the 28 infants used in the initial LC-MALDI discovery experiments. We then validated the urine samples from the 37 infants in the naïve cohort by the MRM method. Three of the candidate urine peptides (FGA1826, FGA1883 and FGA2659) were found to accurately discriminate the medical NEC from the surgical NEC groups in the discovery cohort (FGA1826, p value 7.25×10−4; FGA1883, p value 2.13×10−6; FGA2659, p value 1.49×10−6; FIG. 10A) and the naïve validation cohort (FGA1826, p value 1.07×10−2; FGA1883, p value 1.33×10−6; FGA2659, p value 2.45×10−5; FIG. 10B). Among the three validated FGA peptides, FGA2659 was the marker with maximum abundance and peak discriminating capabilities between medical NEC and surgical NEC.

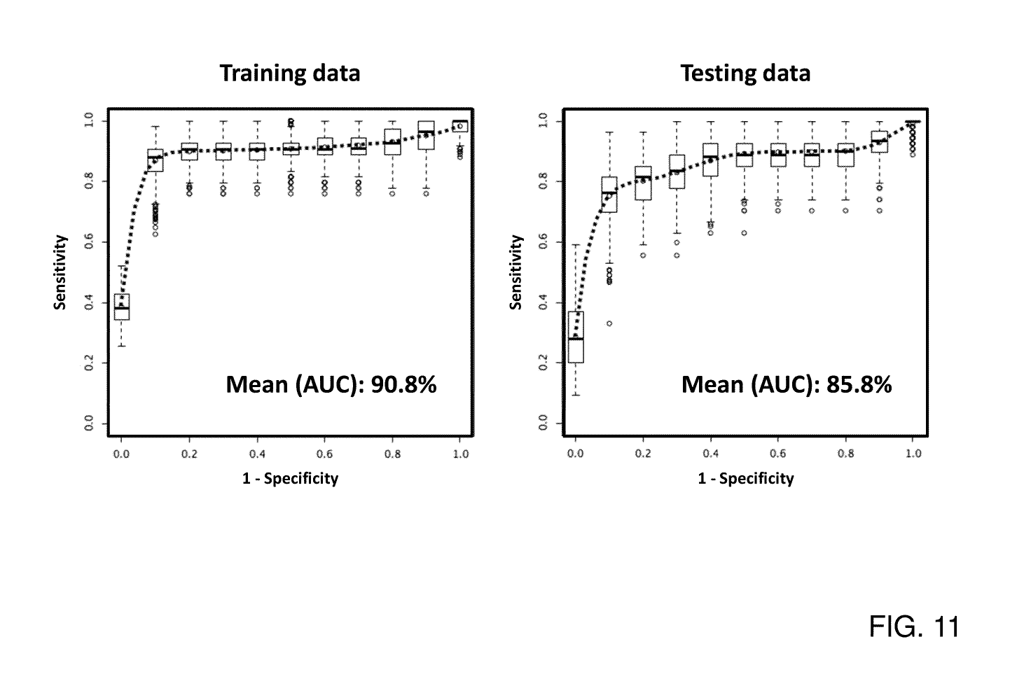

In order to gauge the clinical utility of the FGA peptide biomarker panel, the biomarker discovery cohort and the naïve biomarker validation cohort were assessed by iterative ROC testing (FIG. 11). The ROC curve for the biomarker discovery cohort revealed an AUC of 0.908. The ROC curve analysis of the naïve biomarker validation cohort was 0.858—indicative of a good prognostic test, however, this result was only marginally better than the clinical risk stratification model (AUC 0.817).

Ensemble Algorithm Combining Clinical Parameters and Urine Biomarkers.

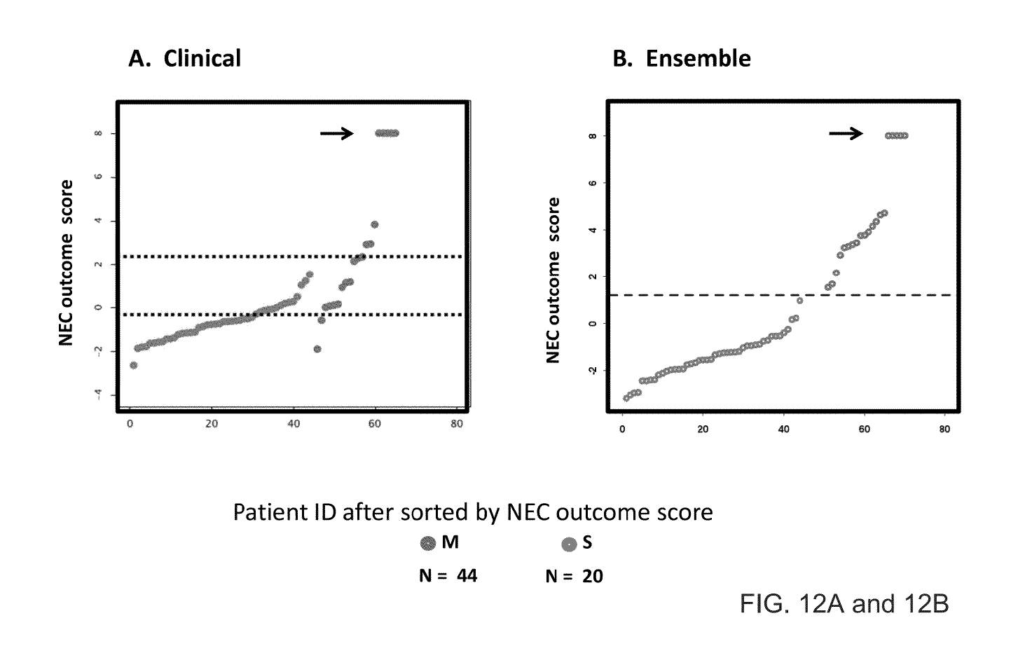

To improve the utility of the clinical parameter-based prognostic algorithm and biomarker panel, we combined the clinical and biomarker classifiers to develop an ensemble model for the prediction of NEC outcomes. We used the 64 subjects that had complete biomarker and clinical data sets (one infant's urine sample had been completely used in the prior biomarker experiments). Infants presenting with pneumoperitoneum on initial abdominal imaging (n=5) were considered to have surgical NEC and were assigned an arbitrarily high NEC ensemble outcome score. The clinical parameter-based prognostic algorithm, when used alone, resulted in significant overlap between the medical NEC and surgical NEC cohorts leaving 39.1% (total n=25/64; medical NEC, n=17/44; surgical NEC, n=8/20) in the indeterminate diagnosis group (FIG. 12A). The combination of the clinical parameter-based prognostic algorithm with the three FGA peptide biomarkers accurately predicted outcome for all infants in the medical NEC and surgical NEC groups (FIG. 12B).

### Discussion

There are currently no reliable prognostic instruments, clinical or biological, that accurately identify infants with progressive NEC prior to the development of irreversible intestinal damage and severe systemic illness. Bell's original staging criteria, with slight modification, are still widely used in the initial diagnosis of infants with NEC. Although Bell's criteria are useful at the time of diagnosis they have limited forecasting ability. We sought to define a novel ensemble prognostic algorithm by combining clinical data with novel urine biomarkers in order to accurately predict the presence of surgical NEC prior to overt clinical manifestation of disease. Risk-stratification by clinical parameters alone revealed an AUC of 0.817 by ROC analysis, while stratification by the biomarker panel alone revealed a slightly better AUC of 0.858. When combined, however, the ensemble algorithm fully discriminated between all infants with medical NEC and surgical NEC.

We have previously demonstrated that urine is a rich source of proteolytically cleaved proteins cleared from plasma by the kidneys and profiling analyses has proven highly informative for urogenital and systemic disease classification (Ling X B, et al. A diagnostic algorithm combining clinical and molecular data distinguishes Kawasaki disease from other febrile illnesses. BMC Med 2010; 9:130-41; Ling X B, et al. Urine peptidomics for clinical biomarker discovery. Adv Clin Chem 2010; 51:181-213; Ling X B, et al. Integrative urinary peptidomics in renal transplantation identifies biomarkers for acute rejection. J Am Soc Nephrol 2010; 21:646-53; Ling X B, et al. Urine peptidomic and targeted plasma protein analyses in the diagnosis and monitoring of systemic juvenile idiopathic arthritis. Clin Proteomics 2010; 6:175-93; Ling X B, et al. Urine peptidomic and targeted plasma protein analyses in the diagnosis and monitoring of systemic juvenile idiopathic arthritis. Clin Proteomics 2010; 6:175-93; Decramer S, de Peredo A G, Breuil B, et al. Urine in clinical proteomics. Mol Cell Proteomics 2008; 7:1850-62; Sigdel T, Ling X B, Lau K, et al. Urinary peptidomic analysis identifies potential biomarkers for acute rejection of renal transplantation. Clin Proteomics 2009; 5:103-13). Importantly, the identified candidate peptide biomarkers in the current study have known biological functions supporting plausible roles in the pathophysiology of NEC. The described FGA peptides, in addition to being quantitatively validated and found to robustly predict disease progression in the ensemble model, contain overlapping sequences—suggesting that they reflect the activity of disease-related coagulation cascade proteases or their inhibitors (Lin Z, et al. Gene expression profiles of human chondrocytes during passaged monolayer cultivation. J Orthop Res 2008; 26:1230-7; Senzaki H. The pathophysiology of coronary artery aneurysms in Kawasaki disease: role of matrix metalloproteinases. Arch Dis Child 2006; 91:847-51; Peng Q, et al. Clinical value of serum matrix metalloproteinase-9 and tissue inhibitor of metalloproteinase-1 for the prediction and early diagnosis of coronary artery lesion in patients with Kawasaki disease. Zhonghua Er Ke Za Zhi 2005; 43:676-80; Gavin P J, et al. Systemic arterial expression of matrix metalloproteinases 2 and 9 in acute Kawasaki disease. Arterioscler Thromb Vasc Biol 2003; 23:576-81; Chua M S, Sarwal M M. Microarrays: new tools for transplantation research. Pediatr Nephrol 2003; 18:319-27; Senzaki H, et al. Circulating matrix metalloproteinases and their inhibitors in patients with Kawasaki disease. Circulation 2001; 104:860-3; Matsuyama T. Tissue inhibitor of metalloproteinases-1 and matrix metalloproteinase-3 in Japanese healthy children and in Kawasaki disease and their clinical usefulness in juvenile rheumatoid arthritis. Pediatr Int 1999; 41:239-45. The parent protein of our peptide biomarkers, fibrinogen A, represents the α chain of the fibrinogen protein. Fibrinogen is cleaved by thrombin during coagulation to form a fibrin thrombus. Thus it is conceivable that this peptide signature reflects the underlying advancing intravascular coagulation that is a distinct hallmark of progressive NEC. In addition, various cleavage products of fibrinogen have been reported to regulate cell adhesion, migration, vasoconstriction and inflammation as well as serve as mitogens for a variety of inflammatory cell types (Herrick S, et al. Fibrinogen. Int J Biochem Cell Biol 1999; 31:741-6; Bennett J S. Platelet-fibrinogen interactions. Ann N Y Acad Sci 2001; 936:340-54; Matsuda M, Sugo T. Structure and function of human fibrinogen inferred from dysfibrinogens. Int J Hematol 2002; 76(Suppl 1):352-60; Lord S T. Fibrinogen and fibrin: scaffold proteins in hemostasis. Curr Opin Hematol 2007; 14:236-41).

The clinical applicability of the prognostic instrument presented here is currently limited by the paucity of existing therapies for NEC. However, immediate clinical utility as a triage instrument is a distinct possibility. High-risk infants could be rapidly transferred to high acuity facilities while transfer could potentially be avoided for those in the low-risk cohort. One could also envision decreased use of serial radiography or shorter duration of empirical antibiotic coverage for low-risk infants. The appropriateness of such practices, however, would require prospective clinical trials prior to widespread implementation.

Importantly, clinical risk stratification is a necessary first step in the development of novel treatment strategies. While it is true that few successful therapies have been developed for NEC, it also remains true that there has never before existed an accurate means to identify the high-risk population. As such, few clear metrics of success have been defined, previous studies have suffered from necessary design flaws, and many potential studies remain infeasible. As an example, early surgery is currently inconceivable given that roughly half of all infants with NEC will improve with non-operative management. An accurate risk-stratification instrument, however, would potentially enable the study of early operation for those at high-risk for disease progression. In the least, a prognostic instrument would provide a basis for the more accurate interpretation of the successes or failures of new therapies as they are developed.

Predictive models of disease progression are most useful when they are able to forecast an unforeseen event, signify a change in clinical trajectory or indicate the necessity for treatment. The findings presented here are a significant step towards these goals. We have shown that a combination of clinical parameters and biomarker analysis enables the early diagnosis of infants at risk for rapid disease progression while also accurately identifying those at low risk. While this model may presently be limited to use as a patient triage instrument, further refinement of the model has the potential to improve the care of infants at high risk for significant morbidity and mortality from NEC and identify those for whom novel prevention and treatment strategies may be useful.

### Example 3

Necrotizing enterocolitis (NEC) is an inflammatory condition of the neonatal gastrointestinal (GI) tract most strongly associated with prematurity and the initiation of enteral feeding. The underlying etiology remains poorly understood, but is thought to be multifactorial, involving factors inherent to the premature neonate and its environment. Specific features believed to be involved in the development of NEC include an underdeveloped GI mucosal barrier, immature innate and humoral immunity, uncoordinated intestinal peristalsis, and pathogenic bacterial overgrowth (Lin et al., Necrotizing enterocolitis: recent scientific advances in pathophysiology and prevention. Seminars in perinatology. 2008; 32:70-82). Despite many advances in neonatal intensive care, NEC continues to be a major source of morbidity and mortality in preterm infants. It is diagnosed in 1% to 5% of all neonatal intensive care unit (NICU) patients with an incidence of up to 15% in infants weighing less than 1500 grams. (Kamitsuka et al., The Incidence of Necrotizing Enterocolitis After Introducing Standardized Feeding Schedules for Infants Between 1250 and 2500 Grams and Less Than 35 Weeks of Gestation. Pediatrics. 2000; 105:379-84; Lemons et al., Very Low Birth Weight Outcomes of the National Institute of Child Health and Human Development Neonatal Research Network, January 1995 Through December 1996. Pediatrics. 2001; 107:e1-e8).

NEC occurs across a spectrum of severity from a mild form that resolves with antibiotics and cessation of feedings (Medical NEC) to a progressive form that leads to intestinal perforation, peritonitis and potentially death (Surgical NEC) (Hintz et al., Neurodevelopmental and Growth Outcomes of Extremely Low Birth Weight Infants After Necrotizing Enterocolitis. Pediatrics. 2005; 117:696-703). Approximately 20% to 40% of all infants diagnosed with NEC progress to require an operation (Henry and Moss, Neonatal necrotizing enterocolitis. Seminars in pediatric surgery. 2008; 17:98-109). While Bell's classification scheme, first introduced in 1978 (Bell et al., Neonatal necrotizing enterocolitis. Therapeutic decisions based upon clinical staging. Annals of Surgery. 1978; 187:1-7), is useful in guiding initial treatment decisions it does not serve as a prognostic instrument of disease progression.

Many prior attempts have been made to identify biologic markers for the early detection of NEC. Breath hydrogen levels, genomic analyses, targeted inflammatory marker detection, and fecal microbiota profiling have all shown initial promise as predictors of high-risk populations, but have achieved limited clinical success for diverse reasons. In the current study we employed an unbiased exploratory proteomics approach to define a urine protein biomarker panel with the ability to enable both timely diagnosis and accurate prognosis for infants with presumed NEC.

### Materials and Methods

Study Design.

This was a multi-institutional, multi-year study with prospective data collection performed from May 1, 2007 to Aug. 1, 2012 by trained personnel at each participating institution. Patient contributions by institution included: Yale-New Haven Children's Hospital (n=42), Johns Hopkins Children's Center (n=27), Texas Children's Hospital (n=25), Lucile Packard Children's Hospital (n=18), and Children's Hospital of Philadelphia (n=7). Informed consent was obtained from the parents of all enrolled subjects. This study was approved by the human subjects' protection program at each participating institution.

All urine samples were obtained from infants treated at one of the collaborating institutions and were collected at the time of initial clinical concern for disease (NEC or sepsis)—a point at which definitive diagnosis was not able to be determined on clinical grounds alone. Patients with a previous diagnosis of NEC or sepsis, a history of prior abdominal surgery, or a known congenital anomaly of the gastrointestinal tract or abdominal wall were excluded from the study. Patient inclusion was ultimately confirmed by the presence of signs specific for NEC by Bell's criteria (pneumatosis intestinalis) or, for the sepsis group, by either positive blood cultures or a clinical syndrome associated with a high probability of infection. Control subjects were identified as premature infants in the NICU without known or suspected inflammatory disease.

The study was conducted in two phases. The ‘discovery phase’ included urine proteomics analysis by non-targeted, liquid chromatography/mass spectrometry (LCMS) with case and control subjects (n=45 NEC, n=12 Sepsis, n=2 Controls) (Ling and Sylvester, Proteomics and biomarkers in neonatology. NeoReviews. 2011:585-591; Ling X B, et al., Urine peptidomics for clinical biomarker discovery. Advances in clinical chemistry. 2010; 51:181-213). To verify the LCMS spectral counts in a proof-of-principle experiment, the CD14 LCMS analyte results were compared to CD14 western blot analysis. For the western blot analysis, CD14 MaxPab mouse polyclonal antibody (B01, Abnova, Taiwan) was used as the primary antibody and a fluorescent-labled secondary antibody was subsequently applied. Gel band intensities were quantified using GelAnalyzer software (http://www.gelanalyzer.com).

The ‘validation phase’ consisted of the analysis of a second, naïve patient cohort (n=40 NEC, n=5 Sepsis, n=15 healthy Controls) for which enzyme-linked immunosorbent assay (ELISA) technology was used to quantify the previously identified urine protein biomarker candidates. All ELISAs were performed according to vendor instructions for the measurement of selected biomarkers in the urine using commercially available kits (Abcam, Mass.; Biolegend Inc., SD; Ebioscience Inc., SD; Fisher Scientific, Ill.; Uscn Life Science Inc., Wuhan, China). The protein analytes' urine abundance was reported as a normalized ratio of the ELISA derived concentration to urinary Creatinine (UCr) concentration to correct for urine biological variations.

Statistical Analyses.

Patient demographic data was analyzed using the Epidemiological calculator (R epicalc package). Student's t test was performed to calculate p values for continuous variables, and Fisher exact test was used for comparative analysis of categorical variables. Hypothesis testing to detect statistical differences in discovered biomarkers was performed using Student's t test (two-tailed) and Mann-Whitney U test (two-tailed), along with local false discovery rate (FDR) (Ling X B, et al., Urine peptidomics for clinical biomarker discovery. Advances in clinical chemistry. 2010; 51:181-213) methods to correct for multiple hypothesis testing issues.

We then performed biomarker feature selection and panel optimization with the aim to develop a multiplexed antibody-based assay for both the diagnosis and prognosis of NEC. This was accomplished using a genetic algorithm (R genalg package) to construct biomarker panels from the validated urine protein biomarkers. Using the validation ELISA data, optimal biomarker panels were identified by testing all possible combinations of the validated urine protein biomarkers while balancing the need for small panel size, accuracy of classification, goodness of class separation (NEC vs. Sepsis, Medical NEC vs. Surgical NEC, NEC vs. Control, and Sepsis vs. Control), and sufficient sensitivity and specificity.

The predictive performance of each biomarker panel analysis was evaluated by ROC curve analysis by plotting the sensitivity vs. 1-specificity (Efron B, et al., Empirical bayes analysis of microarray experiment. J Am Stat Assoc. 2001; 96:1151-60; Zweig and Campbell, Receiver-operating characteristic (ROC) plots: a fundamental evaluation tool in clinical medicine. Clinical chemistry. 1993; 39:561-77). The biomarker panel score was defined as the ratio between the geometric means of the respective up- and down-regulated protein biomarkers. To define the performance of the biomarker panels we chose the coordinates on the ROC curve that represented the “cut-off” point with the best sensitivity and specificity as previously described (Zweig and Campbell, Receiver-operating characteristic (ROC) plots: a fundamental evaluation tool in clinical medicine. Clinical chemistry. 1993; 39:561-77).

### Results

Patient Characteristics.

The patient characteristics are depicted in Table 12. The only characteristic with a statistically significance difference between groups in the discovery cohort was race, with a greater percentage of black infants in the NEC group compared to the Sepsis and Control groups. The characteristics with statistically significance differences between groups in the biomarker validation cohort were gestational age and birth weight, with infants in the Control group tending to have younger gestational ages and lower birth weights than those in the NEC and Sepsis groups. The time between initial clinical concern (i.e. the time of urine sample collection) and confirmed medical NEC, defined as the presence of pneumatosis, was median 32 hours (interquartile range; IQR; 9.5, 66.5). The time between initial clinical concern and confirmation of surgical NEC, defined as the time of laparotomy, peritoneal drain, or death from complication of NEC, was median 48 hours (IQR; 12, 171.5).

TABLE 12
Patient Characteristics
DISCOVERY COHORT (n = 59)

| | NEC | | | | |
|---|---|---|---|---|---|
| | MEDICAL | SURGICAL | | | |
| | NEC | NEC | TOTAL NEC | SEPSIS | CONTROL |
| | (n = 29) | (n = 16) | (n = 45) | (n = 12) | (n = 2) |
| #of Obs. | n = 26 | n = 14 | n = 40 | n = 12 | n = 2 |

Gender

| | | | | | | | | | | |
|---|---|---|---|---|---|---|---|---|---|---|
| Female | 12 | (46.2%) | 7 | (50.0%) | 19 | (47.5%) | 7 | (58.3%) | 2 | (100.0%) |
| Male | 14 | (53.8%) | 7 | (50.0%) | 21 | (52.5%) | 5 | (41.7%) | 0 | (0.0%) |

Race*

| | | | | | | | | | | |
|---|---|---|---|---|---|---|---|---|---|---|
| Asian | 1 | (3.4%) | 0 | (0.0%) | 1 | (5.0%) | 1 | (8.3%) | 2 | (100.0%) |
| Black | 8 | (27.6%) | 5 | (31.2%) | 13 | (28.9%) | 0 | (0.0%) | 0 | (0.0%) |
| White | 16 | (55.2%) | 6 | (37.5%) | 22 | (48.9%) | 11 | (91.7%) | 0 | (0.0%) |
| Unknown | 4 | (13.8%) | 5 | (31.5%) | 9 | (20.0%) | 0 | (0.0%) | 0 | (0.0%) |

Gestational Age (weeks)

| | | | | | |
|---|---|---|---|---|---|
| Median | 28.5 | 28.5 | 28.5 | 28 | 30.5 |
| (IQR) | (27, 32) | (25, 31.8) | (27, 32) | (26.5, 32.5) | (28.2, 32.8) |

Birth Weight (grams)

| | | | | | |
|---|---|---|---|---|---|
| Median | 1095 | 970 | 1070 | 1047.5 | 1840 |
| (IQR) | (937.5, 1952) | (740.5, 1771.2) | (850, 1947.8) | (840, 1927.5) | (1350, 2330) |

Birth Length (cm)

| | | | | | |
|---|---|---|---|---|---|
| Median | 36 | 34.5 | 35.75 | 37 | 41 |
| (IQR) | (33, 42) | (33, 43.2) | (33, 43.2) | (32, 43) | (34, 48) |

Birth Head Circumference (cm)

| | | | | | |
|---|---|---|---|---|---|
| | | | | | 28.5 |
| Median | 26 | 24.5 | 26 | 24.5 | (26.2, 30.8) |
| (IQR) | (24.5, 31) | (23.5, 27.9) | (23.5, 30.2) | (24, 28.8) | |

VALIDATION COHORT (n = 59)

| NEC | | | | |
|---|---|---|---|---|
| MEDICAL | SURGICAL | | | |
| NEC | NEC | TOTAL NEC | SEPSIS | CONTROL |
| (n = 30) | (n = 10) | (n = 40) | (n = 5) | (n = 15) |

Gender

| | | | | | | | | | | |
|---|---|---|---|---|---|---|---|---|---|---|
| Female | 16 | (53.3%) | 2 | (20.0%) | 18 | (45.0%) | 3 | (60.0%) | 6 | (40.0%) |
| Male | 14 | (46.7%) | 8 | (80.0%) | 22 | (55.0%) | 2 | (40.0%) | 9 | (60.0%) |

Race

| | | | | | | | | | | |
|---|---|---|---|---|---|---|---|---|---|---|
| Asian | 2 | (6.7%) | 0 | (0.0%) | 2 | (5.0%) | 0 | (0.0%) | 0 | (0.0%) |
| Black | 13 | (43.3%) | 3 | (30.0%) | 16 | (40.0%) | 3 | (60.0%) | 7 | (46.7%) |
| White | 13 | (43.3%) | 6 | (60.0%) | 19 | (47.5%) | 1 | (20.0%) | 7 | (46.7%) |
| Unknown | 2 | (6.7%) | 1 | (10.0%) | 3 | (7.5%) | 1 | (20.0%) | 1 | (6.7%) |

Gestational Age* (weeks)

| | | | | | |
|---|---|---|---|---|---|
| Median | 30 | 27.5 | 29.5 | 28 | 26 |
| (IQR) | (27, 33) | (25, 32) | (27, 32.5) | (26, 31.5) | (25, 27.5) |

Birth Weight* (grams)

| | | | | | |
|---|---|---|---|---|---|
| Median | 1265 | 1285 | 1265 | 950 | 730 |
| (IQR) | (935, 1873.5) | (796.5, 1912.5) | (907, 1943.8) | (900, 961) | (632.5, 1937.5) |

Birth Length (cm)

| | | | | | |
|---|---|---|---|---|---|
| Median | 37 | 34.5 | 37 | 34 | 33.8 |
| (IQR) | (34.1, 41.8) | (32, 42.8) | (32.9, 42.2) | (31, 36) | (32, 36) |

Birth Head Circumference (cm)

| | | | | | |
|---|---|---|---|---|---|
| Median | 27.2 | 24.4 | 27 | 24.2 | 23 |
| (IQR) | (25, 30.5) | (23, 28) | (23.9, 30.1) | (23.4, 24.6) | (21.8, 26) |

Biomarker Discovery (LCMS).

LCMS analysis of the 59 infants in the biomarker discovery cohort revealed thirteen candidate proteins with potentially relevant biologic roles: alpha-2-macroglobulin-like protein 1 (A2ML1), apolipoprotein CIII (APO-CIII), complement component protein 3 (C3), caspase protein 8 (CASP8), cluster of differentiation protein 14 (CD14), cystatin 3 (CST3), fibrinogen alpha chain (FGA), kininogen protein 1 (KNG1), lectin manose-binding protein 2 (LMAN2), pigment epithelium-derived factor (PEDF), Pmp-like secreted protein 2 (PLS2), retinol binding protein 4 (RET4), and vasolin (VASN).

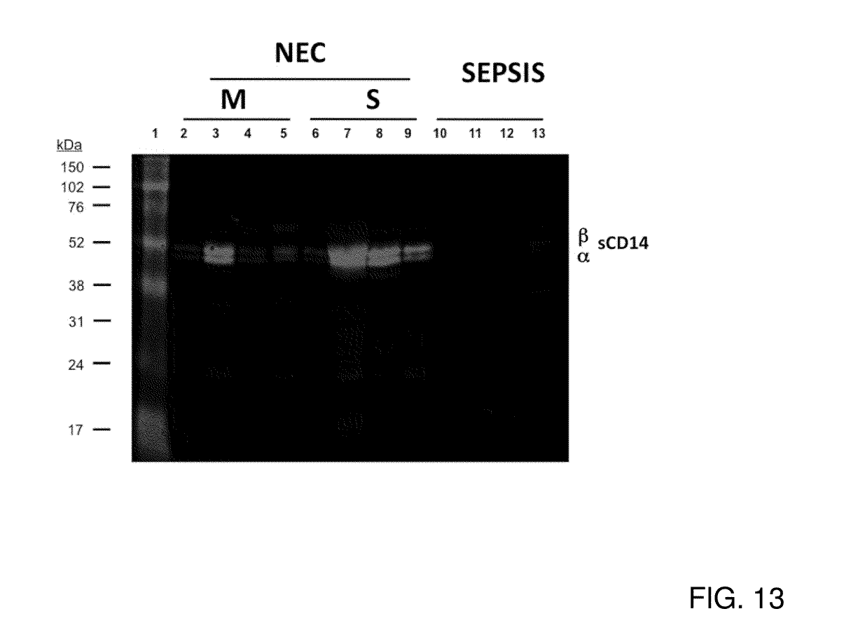

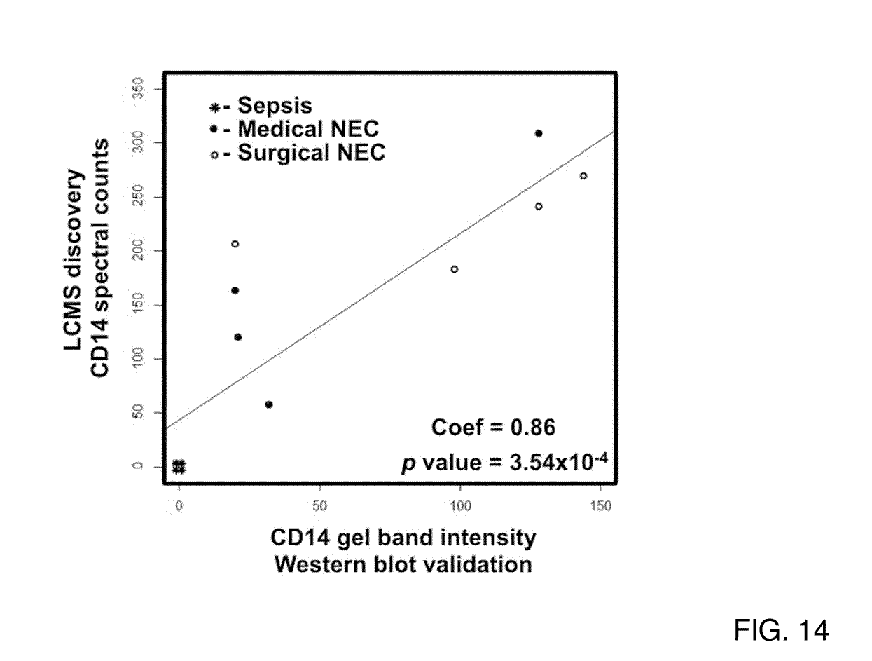

As a verification of the LCMS discovery approach, the differential presence of CD14, a pattern recognition receptor (PRP), was confirmed by Western blot analysis comparing Medical NEC, Surgical NEC and Sepsis urine samples (FIG. 13). Western blot revealed the alpha-form and beta-form of soluble CD14, both of which are known to be up-regulated in the plasma of adults experiencing pro-inflammatory conditions (Fingerle-Rowson G, et al., Down-regulation of surface monocyte lipopolysaccharide-receptor CD14 in patients on cardiopulmonary bypass undergoing aorta-coronary bypass operation. The Journal of thoracic and cardiovascular surgery. 1998; 115:1172-8). LCMS spectral counts were then plotted against CD14 Western blot band intensity revealing a correlation coefficient of 0.86 (p<0.001; FIG. 14) with the more severe pathology (Surgical NEC) displaying higher levels of CD14 expression by both analytical methods.

Biomarker Validation (ELISA).

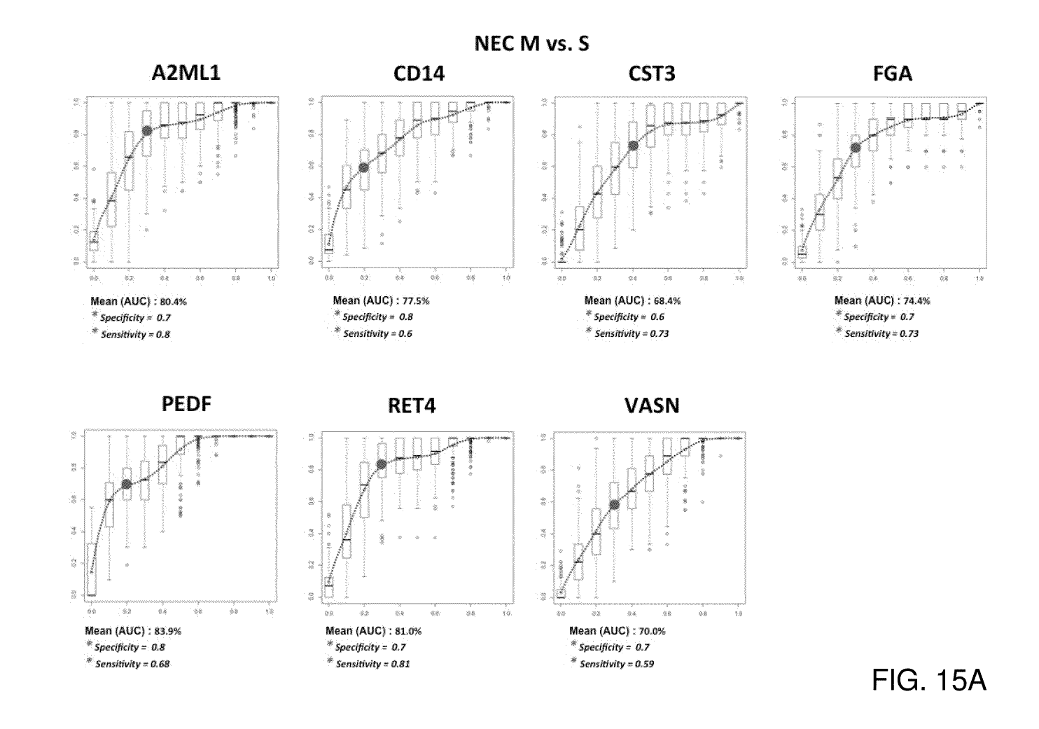

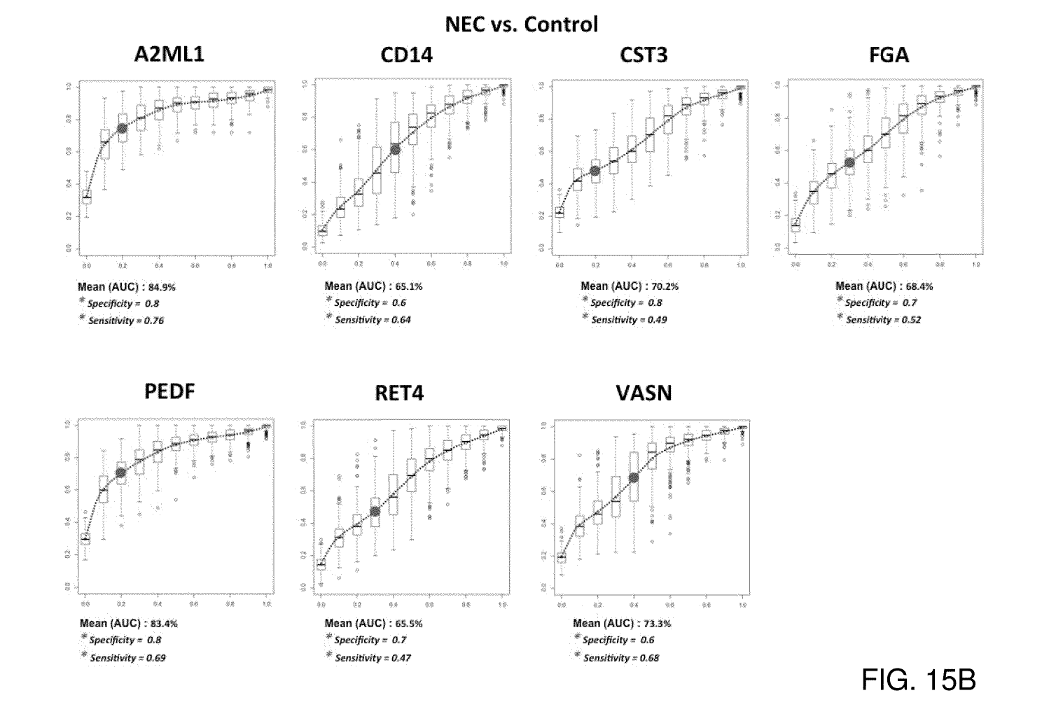

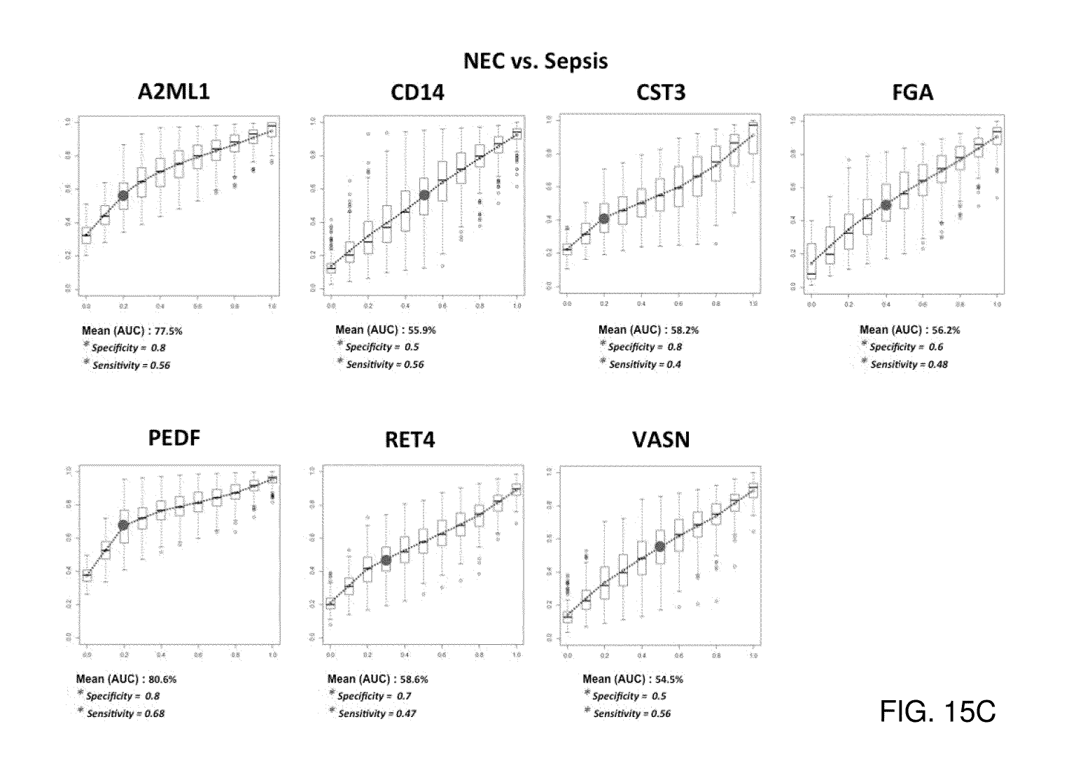

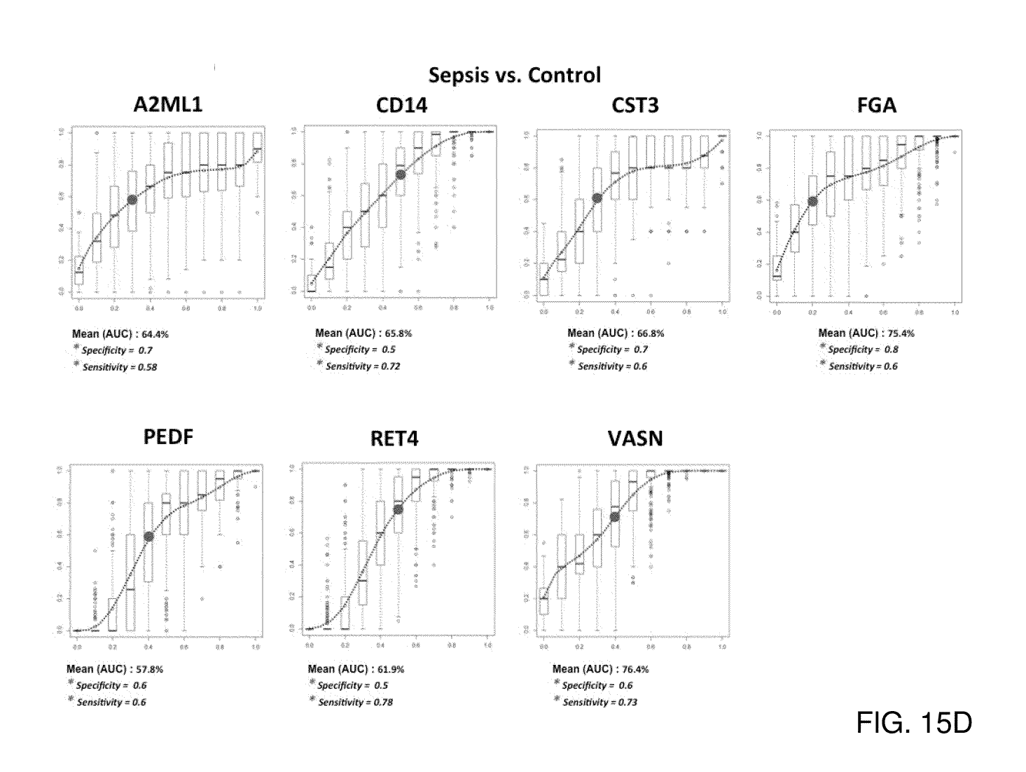

The urine samples from the 60 infants in the validation cohort were used for ELISA-based validation of the candidate biomarkers. Commercially available ELISA assays for the 13 candidate biomarkers were utilized. Seven of the 13 LC-MS candidate biomarkers were quantitatively validated (Two-tailed Mann-Whitney tests p<0.05; Table 13 and Table 14) and consistently shared the same trend of up- or down-regulation between case and control samples when comparing discovery LCMS and validation ELISA results. Additionally, individual ROC curves were plotted for each validated analyte and the point of intersection for optimal sensitivity and specificity was computed, demarcated (FIG. 15A-D) and reported (Table 15).

TABLE 13
ELISA biomarker validation by Mann-Whitney U test

| | Mann-Whitney U test p value | | | |
|---|---|---|---|---|
| | | NEC vs. | NEC vs. | Sepsis vs. |
| Analyte | NEC M vs. S | Sepsis | Control | Control |
| A2ML1 | 0.02 * | 0.08 | 1.40 × 10^{-4 **} | 0.50 |
| CD14 | 0.02 * | 0.77 | 0.12 | 0.35 |
| CST3 | 0.12 | 0.58 | 0.03 * | 0.35 |
| FGA | 0.02 * | 0.80 | 0.06 | 0.16 |
| PEDF | 1.82 × 10^{-3 **} | 0.03 * | 2.23 × 10⁻⁴ ^{**} | 0.67 |
| RET4 | 6.89 × 10^{-3 *} | 0.64 | 0.11 | 0.50 |
| VASN | 0.09 | 0.80 | 0.02 * | 0.12 |

TABLE 14
Validated biomarker levels by pathologic group

| | | NEC | | | | | | | | | |
|---|---|---|---|---|---|---|---|---|---|---|---|
| | | M | | S | | M + S | | Sepsis | | Control | |
| | | Median | Mean | Median | Mean | Median | Mean | Median | Mean | Median | Mean |
| Analyte | Unit | (IRQ) | (SD) | (IRQ) | (SD) | (IRQ) | (SD) | (IRQ) | (SD) | (IRQ) | (SD) |
| A2ML1 | Analyte/Cr | 61.55 | 174.03 | 3.79 | 22.25 | 28.28 | 138.61 | 3.32 | 5.30 | 1.68 | 2.71 |
| | (ng/mg) | (14.12, | (346.64) | (1.40, | (47.81) | (3.79, | (309.67) | (1.55, | (6.37) | (0.96, | (3.03) |
| | | 166.37) | | 9.51) | | 130.13) | | 9.06) | | 3.36) | |
| CD14 | Analyte/Cr | 174.40 | 451.76 | 895.49 | 2740.24 | 212.66 | 979.87 | 186.53 | 367.62 | 89.44 | 295.63 |
| | (ng/mg) | (84.74, | (726.18) | (231.43, | (5004.98) | (110.28, | (2574.92 | (100.67, | (361.03 | (39.14, | (375.31) |
| | | 524.22) | | 2601.20 | | 679.12) | | 655.39 | | 574.68) | |
| CST4 | Analyte/Cr | 43.70 | 215.50 | 227.20 | 355.27 | 87.30 | 248.39 | 94.22 | 81.14 | 31.14 | 51.12 |
| | (ng/mg) | (21.30, | (416.28) | (120.54, | (352.39) | (23.16, | (401.55) | (59.22, | (52.93) | (12.89, | (47.37) |
| | | 225.23) | | 605.62) | | 239.16) | | 111.59) | | 86.68) | |
| FGA | Analyte/Cr | 15.78 | 74.18 | 69.50 | 408.39 | 21.57 | 157.73 | 29.06 | 95.71 | 15.52 | 22.67 |
| | (ng/mg) | (9.26, | (143.97) | (46.25, | (862.69) | (9.95, | (456.77) | (15.51, | (149.33 | (4.23, | (35.07) |
| | | 33.81) | | 237.97) | | 97.57) | | 175.91) | | 23.63) | |
| PEDF | Analyte/Cr | 4.40 | 66.05 | 122.04 | 225.45 | 8.60 | 115.86 | 111.66 | 212.40 | 217.34 | 378.60 |
| | (ng/mg) | (1.57, | (228.22) | (7.14, | (309.84) | (2.79, | (262.27) | (100.56, | (225.50 | (57.49, | (411.31) |
| | | 25.31) | | 257.70) | | 105.75) | | 134.47 | | 491.52) | |
| RET4 | Analyte/Cr | 417.89 | 642.35 | 1121.99 | 5549.31 | 512.72 | 1796.93 | 454.38 | 463.24 | 298.60 | 406.36 |
| | (ng/mg) | (188.59, | (846.82) | (898.48, | (12299.56 | (197.95, | (6090.69 | (337.35, | (220.19 | (115.29, | (357.47 |
| | | 655.45 | | 2083.34 | | 1115.57 | | 655.21 | | 692.03 | |
| VASN | Analyte/Cr | 23.93 | 97.17 | 9.84 | 17.04 | 19.99 | 78.68 | 13.67 | 26.40 | 2.74 | 11.04 |
| | (ng/mg) | (9.78, | (163.15) | (6.32, | (18.11) | (9.04, | (146.81) | (10.70, | (24.62) | (0.54, | (12.62) |
| | | 129.94) | | 21.43) | | 52.85) | | 43.44) | | 22.83) | |

TABLE 15
Individual biomarker inter-cohort testing characteristics.

| | NEC M vs S | | | NEC vs Control | | | NEC vs Sepsis | | | Sepsis vs Control | | |
|---|---|---|---|---|---|---|---|---|---|---|---|---|
| | ROC | Sensi- | Specif- | ROC | Sensi- | Specif- | ROC | Sensi- | Specif- | ROC | Sensi- | Specif- |
| Analyte | AUC | tivity* | icity* | AUC | tivity* | icity* | AUC | tivity* | icity* | AUC | tivity* | icity* |
| A2ML1 | 80.40% | 0.80 | 0.70 | 84.90% | 0.76 | 0.80 | 77.50% | 0.56 | 0.80 | 0.78 | 0.56 | 0.80 |
| CD14 | 77.50% | 0.60 | 0.80 | 65.10% | 0.64 | 0.60 | 55.90% | 0.56 | 0.50 | 0.56 | 0.56 | 0.50 |
| CST4 | 68.40% | 0.73 | 0.60 | 70.20% | 0.49 | 0.80 | 58.20% | 0.4 | 0.80 | 0.58 | 0.40 | 0.80 |
| FGA | 74.40% | 0.73 | 0.70 | 68.40% | 0.52 | 0.70 | 56.20% | 0.48 | 0.60 | 0.56 | 0.48 | 0.60 |
| PEDF | 83.90% | 0.68 | 0.80 | 83.40% | 0.69 | 0.80 | 80.60% | 0.68 | 0.80 | 0.58 | 0.60 | 0.60 |
| RET4 | 81.00% | 0.81 | 0.70 | 65.50% | 0.47 | 0.70 | 58.60% | 0.47 | 0.70 | 0.62 | 0.78 | 0.50 |
| VASN | 70.00% | 0.59 | 0.70 | 73.30% | 0.68 | 0.60 | 54.50% | 0.56 | 0.50 | 0.76 | 0.73 | 0.60 |

*The optimal sensitivity and specificity point along the ROC curve.

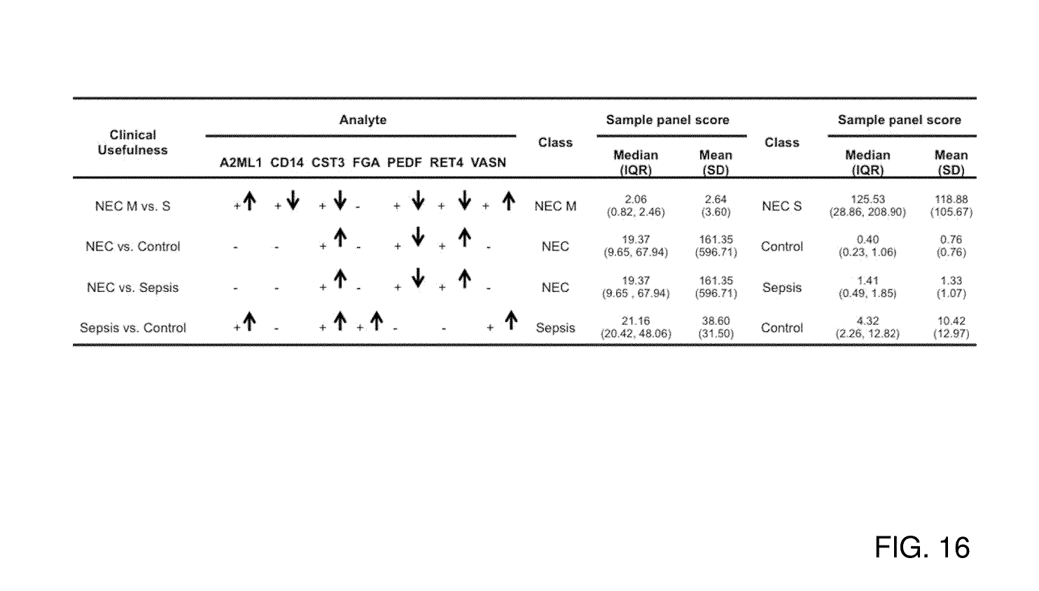

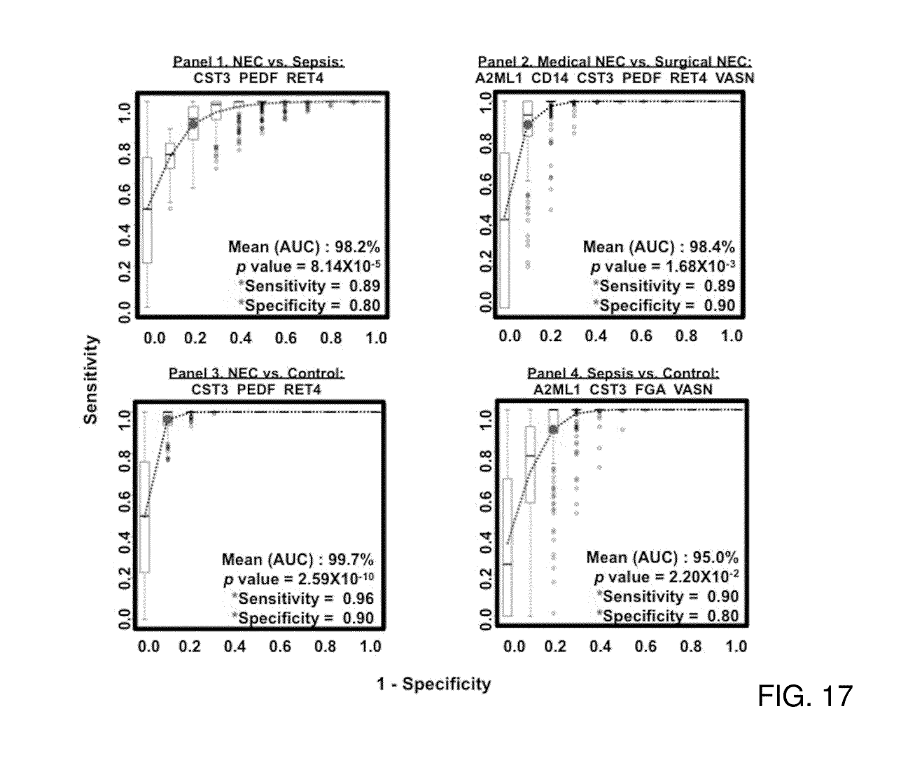

The genetic algorithm panel construction process led to the design of four distinct biomarker panels with complete separation between NEC vs. Sepsis, Medical NEC vs. Surgical NEC, NEC vs. Control, and Sepsis vs. Control (FIG. 16; FIG. 17). These biomarker panels are non-redundant indicative of their non-inclusive relationships.

Importantly, each biomarker panel was able to differentiate between the groups with sensitivities ranging from 0.89-0.96 and specificity ranging from 0.80-0.90 (FIG. 17). Not surprisingly, the panels assessing infants with diagnoses more closely related in severity of inflammation had lower sensitivity (NEC vs. Sepsis, 0.89; and Medical NEC vs. Surgical NEC, 0.89) compared to the panels including the Controls (NEC vs. Control, 0.96; and Sepsis vs. Control, 0.90).

### Discussion

Considerable effort has been directed toward the identification of biomarkers of NEC given the inability to predict the ultimate course of disease based on clinical parameters alone (Moss R L, et al., Clinical parameters do not adequately predict outcome in necrotizing enterocolitis: a multi-institutional study. Journal of perinatology: official journal of the California Perinatal Association. 2008; 28:665-74). Exploratory proteomics enables the unbiased identification of candidate biomarkers prior to clinical manifestation of disease. Urine biomarker panels, specifically, hold the potential to provide low risk, low cost facilitation of clinical decision-making. The urine protein biomarkers described in the current study enabled the accurate diagnosis of NEC amongst a population of infants with NEC, infants with non-NEC sepsis, and non-infected premature infants. In addition, these biomarkers showed potential prognostic value, as they were also able to accurately differentiate between infants with Medical NEC and those with Surgical NEC.

Many prior studies have investigated the diagnostic capabilities of targeted biomarkers for NEC. Epidermal growth factor (EGF) (Helmrath M A, et al., Epidermal growth factor in saliva and serum of infants with necrotising enterocolitis. Lancet. 1998; 351:266-7; Shin C E, et al., Diminished epidermal growth factor levels in infants with necrotizing enterocolitis. Journal of pediatric surgery. 2000; 35:173-6; discussion 7), inter-alpha inhibitor proteins (iaips) (Lim Y P, et al., Correlation between mortality and the levels of inter-alpha inhibitors in the plasma of patients with severe sepsis. J Infect Dis. 2003; 188:919-26; Chaaban H, et al., The role of inter-alpha inhibitor proteins in the diagnosis of neonatal sepsis. J. Pediatr. 2009; 154:620-2 e1; Baek Y W, et al., Inter-alpha inhibitor proteins in infants and decreased levels in neonatal sepsis. J. Pediatr. 2003; 143:11-5; Chaaban H, et al., Inter-alpha inhibitor protein level in neonates predicts necrotizing enterocolitis. J. Pediatr. 2010; 157:757-61), intestinal fatty acid-binding protein (1-FABP) (Lieberman J M, et al., Human intestinal fatty acid binding protein: report of an assay with studies in normal volunteers and intestinal ischemia. Surgery. 1997; 121:335-42; Edelson M B, et al., Plasma intestinal fatty acid binding protein in neonates with necrotizing enterocolitis: a pilot study. J Pediatr Surg. 1999; 34:1453-7) and fecal calprotectin (Reisinger K W, et al., Noninvasive measurement of fecal calprotectin and serum amyloid A combined with intestinal fatty acid-binding protein in necrotizing enterocolitis. J Pediatr Surg. 2012; 47:1640-5) have all been implicated as potential biomarkers of NEC in human infants. Additionally, a number of interleukins and other inflammatory factors have been found to be either up-regulated (IL 1, 6, 8, and 12, tumor necrosis factor-alpha, interferon, and platelet activating factor), down-regulated or temporally correlated with the severity of disease (IL 4, 10, and 11) in infants with NEC or other inflammatory conditions of infancy (Martin and Walker, Intestinal immune defences and the inflammatory response in necrotising enterocolitis. Semin Fetal Neonatal Med. 2006; 11:369-77; Edelson M B, et al., Circulating pro- and counterinflammatory cytokine levels and severity in necrotizing enterocolitis. Pediatrics. 1999; 103:766-71; Caplan M S, et al., Role of platelet activating factor and tumor necrosis factor-alpha in neonatal necrotizing enterocolitis. J. Pediatr. 1990; 116:960-4; Viscardi R M, et al., Inflammatory cytokine mRNAs in surgical specimens of necrotizing enterocolitis and normal newborn intestine. Pediatr Pathol Lab Med. 1997; 17:547-59). Despite promising results, no single biomarker has proven to be useful as a stand-alone diagnostic test in clinical practice. In contrast, the current study made use of a non-targeted, exploratory approach to identify several candidate biomarkers. The biomarker panels were subsequently validated on a naïve population with relatively strong diagnostic (NEC vs. Sepsis; mean AUC 98.2%, sensitivity 0.89, specificity 0.80) and prognostic (Medical NEC vs. Surgical NEC; mean AUC 98.4%, sensitivity 0.89, specificity 0.90) capabilities.

Importantly, many of the validated biomarkers have potential physiologic bases for their association with NEC. A1pha-2macroglobulin (A2M, which shares significant homology with A2ML1) and FGA are both components of the coagulation cascade—a potentially significant finding given that coagulation necrosis is a common pathological finding in NEC resection specimens. VASN is an inhibitor of TGF-beta, and has been found to be down regulated following vascular injury (Ikeda Y, et al., Vasorin, a transforming growth factor beta-binding protein expressed in vascular smooth muscle cells, modulates the arterial response to injury in vivo. Proc Natl Acad Sci USA. 2004; 101:10732-7)—a finding consistent with lower urine levels of VASN in the Surgical NEC cohort. The PRP CD14 is a regulator of the innate immune system that plays a role in the response to bacterial lipopolysaccharide (LPS) potentially explaining its elevation in the Surgical NEC cohort, a patient group with more extensive bowel injury and thus bacterial invasion. CST3 has been described as a biomarker for acute kidney injury (Urbschat A, et al., Biomarkers of kidney injury. Biomarkers: biochemical indicators of exposure, response, and susceptibility to chemicals. 2011; 16 Suppl 1:S22-30) likely explaining its presence in higher levels in the urine as systemic disease progresses. While these associations are intriguing, further investigation is needed to identify causal relationships and to provide further biologic insight.

This study demonstrates the utility of unbiased biomarker discovery platforms in which proteins with correlated and potentially causal relationships to the pathophysiology of disease can be identified. The clinical potential of the described biomarker panel was highlighted by the validation on a naïve population while the inclusion of the Sepsis group in addition to the non-infected Control group confirmed that the identified biomarkers were not simply markers of a generic pro-inflammatory state.

The use of an unbiased exploratory proteomics approach to identify urine biomarkers for NEC led to the development of a panel of validated proteins that demonstrate promise as a clinically useful instrument. The incorporation of additional targeted biomarkers along with patient-specific clinical information will likely strengthen the utility of the described biomarkers and is an important area of ongoing investigation. Thus, it appears likely that a biomarker-based instrument will lead to more efficient diagnosis, more timely intervention, and improved outcomes for infants affected by one of the most common and debilitating diseases of prematurity.

### Example 4

### Bottom-Up Urine Proteomics Discovered an Eleven-Protein Biomarker Panel that Effectively Discriminates NEC M from S Subjects

71 NEC samples—47 NEC M and 24 S subjects—were analyzed by mass spectrometry (MS) based urine proteome profiling using a bottom up approach. Cross validation and false discovery (FDR) guided feature selection analysis found a eleven-protein panel (PLSL, LMAN2, OSTP/OPN, APOA4, CO8G, SAP, ANGT, CD14, FIBA, PROF1, PEDF) (FIG. 11), which effectively classified the NEC S samples (PLSL and LMAN2 elevated) and M samples (OSTP/OPN, APOA4, CO8G, SAP, ANGT, CD14, FIBA, PROF1, and PEDF elevated) with overall 85.9% accuracy (P value 2.6×10⁻⁸, ROC AUC 92.3% (FIG. 12). Intriguingly, several of these proteins have known biologic functions that may be related to the pathogenesis of progressive NEC and therefore reflect the underlying biology. The PRP CD14 and immune-modulating properties of PEDF were discussed above. Additionally, osteopontin (OSTP/OPN) is a phosphoprotein with a range of described biologic functions including as a pro-inflammatory cytokine for monocytes and macrophages, as well inhibitory of macrophage nitric oxide production. Fibrinogen A (FIBA), a potent member of the coagulation cascade appears to be highly expressed in NEC S class consistent with the high level of coagulative and consumptive necrosis that occurs in advanced cases of NEC (NEC S). Most intriguingly, we also found two peptide fragments from FIBA in the 36-member classifier for progressive NEC above, thus providing further support for the involvement of this molecule in NEC progression and its utility as a biomarker of progressive disease (NEC S).

### Example 5

### Bottom-Up Urine Proteomics Discovered a Seven-Protein Biomarker Panel that Effectively Discriminates NEC from Sepsis Subjects

We sought to identify protein biomarkers of NEC that exist in the urine of infants at the time of first clinical suspicion of either NEC or sepsis. An un-biased, high-throughput proteomic discovery approach was taken utilizing subject samples that were obtained by the NEC Consortium. 71 NEC and 13 Sepsis urine samples underwent mass spectrometry (MS) based urine proteome profiling using a bottom up approach. Each proteome was fragmented by trypsin digestion. Full mass spectrometry scan was acquired on an LTQ FTMS, which was followed by MS/MS analysis. Protein identification was performed by searching Swiss-Prot database. Quantification of proteins in different samples was done by means of spectral counting, implementing the recent S1N algorithm. From the MSMS protein identifications, a separate list of proteins was created for each sample, and the lists were then compared to find differentially expressed proteins. For any given protein, the significance of the relative abundance between NEC and Sepsis groups was computed by Student's T test. Urine proteins with low P values discriminating NEC and Sepsis were explored by exploratory box-whisker plot analysis. Cross validation and false discovery (FDR)guided feature selection analysis revealed a seven-protein panel (CD14, SAP1, PEDF, ftsY, PROC, MAP1B, CSN5) (FIG. 11) that effectively classified the NEC and Sepsis samples with overall 95.2% accuracy (P value 1.9×10⁻⁹, ROC AUC 93% FIG. 10). Among the identified proteins as biomarkers of NEC include several that may be of particular interest given their described biologic functions and the prevailing hypothesis of NEC etiology that includes enteric bacterial invasion of the newborn gut and the inciting inflammatory cascade that results in coagulative necrosis. Perhaps most interesting is CD14, an integral part of the innate immune system as a pattern recognition receptor (PRP) that acts as a co-receptor along with Toll-like receptor 4 (TLR4) and has been implicated as causative of NEC. Although the primary ligand of CD14 is bacterial LPS, it also recognizes other pathogen associated molecular patterns. CD14 exists in two forms including a soluble form (sCD14) that can be shed or secreted from enterocytes. In addition, PEDF (pigment epithelial derived factor) is a serine protease glycoprotein that is known to effect macrophage function through PPAR* and may therefore play a role in modulating NEC associated inflammation.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of diagnosing NEC in a patient, the method comprising:
a. detecting the level in the urine of protein encoded by one or more NEC-Dx genes to obtain an NEC-Dx signature;
b. comparing the NEC-Dx signature to a reference NEC-Dx signature; and
c. employing the results of the comparison to provide an NEC diagnosis to the patient.

2. The method according to claim 1, wherein the one or more NEC-Dx genes is selected from the group consisting of SAP1, PEDF, Q6ZUQ4, OBFC2B, COL11A2, NBEAL2, GRASP, HUWE1, COL1A2, HOXD3, DSG4, KRTAP5-11, Y1020, FGA, UMOD, CTAPIII/PPBP, SAA1, B2M, TTR, OSTP/OPN, APOA4, C08G, ANGT, FIBA, PROF1, PLSL, LMAN2, CST3 and RET4.

3. The method according to claim 1, further comprising obtaining an NEC clinical score, wherein the comparing comprises comparing the NEC-Dx signature and the NEC clinical score to a reference NEC-Dx signature and a reference NEC-Dx clinical score, and the employing comprises employing the results of the comparisons to provide a diagnosis of NEC.

4. The method according to claim 1, wherein the patient is suspected of having NEC, intestinal perforation (IP), or sepsis.

5. A method of diagnosing sepsis in a patient, the method comprising:
a. detecting the level in the urine of protein encoded by one or more sepsis-Dx genes to obtain a sepsis-Dx signature;
b. comparing the sepsis-Dx signature to a reference sepsis-Dx expression signature; and
c. employing the results of the comparison to provide a sepsis diagnosis to the patient.

6. The method according to claim 5, wherein the one or more sepsis-Dx genes selected from the group consisting of ftsy, PROC, MAP1B, CSN5, A2ML1, CST3, FGA, PEDF, and VASN.

7. The method according to claim 5, wherein the patient is suspected of having NEC or sepsis.

8. A method of providing a prognosis for a patient with NEC or predicting responsiveness of a patient with NEC to medical therapy, the method comprising:
a. detecting the level in the urine of protein encoded by one or more NEC-M/S genes to obtain an NEC-M/S signature;
b. comparing the NEC-M/S signature to a NEC-M/S reference signature; and
c. employing the results of the comparison to provide a prognosis for the patient or predict responsiveness of the patient to medical therapy.

9. The method according to claim 8, wherein the one or more NEC-M/S genes is selected from the group consisting of Q6ZUQ4, OBFC2B, COL11A2, NBEAL2, GRASP, HUWE1, COL1A2, HOXD3, DSG4, KRTAP5-11, Y1020, FGA, UMOD, OSTP/OPN, APOA4, CO8G, SAP1, ANGT, CD14, FIBA, PROF1, PEDF, PLSL, LMAN2, CD14, CST3, RET4/RBP4, A2ML1, and VASN.

10. The method according to claim 9, wherein the one or more NEC-M/S genes comprises FGA, and the FGA is detected by detecting one or more FGA peptides selected from the group consisting of DEAGSEADHEGTHSTKR, DEAGSEADHEGTHSTKRG, and DEAGSEADHEGTHSTKRGHAKSRPV.

11. The method according to claim 8, wherein the patient is diagnosed as having NEC.

12. The method according to claim 8, wherein the medical therapy is antibiotics and nothing by mouth.

13. The method according to claim 8, further comprising the step of obtaining an NEC clinical score, wherein the comparing comprises comparing the NEC-M/S signature and the NEC clinical score to a reference NEC-M/S signature and a reference NEC clinical score, and the employing comprises employing the results of the comparisons to provide a prognosis or predict responsiveness of an NEC patient to medical therapy.

14. A kit for diagnosing a patient with NEC, diagnosing a patient with sepsis, providing a prognosis for a patient with NEC or predicting responsiveness of a patient with NEC to medical therapy, the kit comprising:
a detection reagent for the detection of one or more proteins encoded by one or more NEC-Dx, sepsis-Dx, and/or NEC-M/S genes, and
a NEC-Dx signature reference, sepsis-Dx signature reference, and/or NEC-M/S signature reference.

15. The kit according to claim 14, wherein the one or more NEC-M/S genes comprises FGA, wherein the detection reagent detects one or more peptides selected from the group consisting of DEAGSEADHEGTHSTKR, DEAGSEADHEGTHSTKRG, and DEAGSEADHEGTHSTKRGHAKSRPV.
